(19) United States
(12) Patent Application Publication
(10) Pub. No.: US 2021/0283261 A1
(43) Pub. Date: Sep. 16, 2021

(54) Compositions and Methods for Treating ALK-Mediated Cancer

(71) Applicants: ICAHN SCHOOL OF MEDICINE AT MOUNT SINAI, New york, NY (US); THE UNIVERSITY OF NORTH CAROLINA AT CHAPEL HILL, Chapel Hill, NC (US)

(72) Inventors: Jian Jin, New York, NY (US); Yue Xiong, Chapel Hill, NC (US); Jing Liu, New York, NY (US); Chengwei Zhang, New York, NY (US); Xiaoran Han, Chapel Hill, NC (US); He Chen, New York, NY (US); Pu Wang, Chapel Hill, NC (US)

(21) Appl. No.: 16/769,326

(22) PCT Filed: Dec. 4, 2018
(86) PCT No.: PCT/US2018/063847

§ 371 Date: Jun. 3, 2020

Related U.S. Application Data
(60) Provisional application No. 62/594,959, filed on Dec. 5, 2017.

(51) Int. Cl. A61K 47/54; A61K 31/506; C07D 401/14; C07D 417/14; G01N 33/50

(52) U.S. Cl. CPC A61K 47/545; A61K 31/506; G01N 2333/912; C07D 417/14; G01N 33/5011; C07D 401/14

(57) ABSTRACT

Heterobifunctional small molecules including anaplastic lymphoma kinase (ALK) ligand conjugated to a degradation/disruption tag through a linker, which selectively degrade/disrupt ALK, ALK fusion proteins, and/or ALK mutant proteins, and compositions and methods of using such degraders/disruptors to treat ALK-mediated cancer are provided.

### TECHNICAL FIELD

This disclosure relates to bivalent compounds (e.g., bi-functional small molecule compounds) which selectively degrade and/or disrupt anaplastic lymphoma kinase (ALK), compositions comprising one or more of the bivalent compounds, and to methods of use thereof for the treatment of ALK-mediated cancer in a subject in need thereof. The disclosure also relates to methods for designing such bivalent compounds.

### BACKGROUND OF THE INVENTION

Anaplastic lymphoma kinase (ALK), a receptor tyrosine kinase, belongs to the insulin receptor kinase subfamily and plays an important role in the development of the brain and exerts its effects on specific neurons in the nervous system (Iwahara et al., 1997; Morris et al., 1997). ALK was originally discovered through the identification of a 2;5 chromosomal translocation in anaplastic large-cell non-Hodgkin's lymphoma (ALCL) (Morris et al., 1994). The nucleophosmin (NPM)-ALK fusion protein produced by this rearrangement results in a constitutively active, oncogenic tyrosine kinase (Morris et al., 1994; Shiota et al., 1994).

Oncogenic activation of ALK is involved in the initiation and progression of multiple cancer types, including neuroblastoma, lymphomas, non-small-cell lung cancer (NSCLC), colon carcinoma, renal cell carcinoma (RCC), breast cancer, Anaplastic thyroid cancer (ATC), ovarian cancer and esophageal squamous cell carcinoma (ESCC). ALK can be oncogenically activated by different mechanisms, including translocation, mutation and amplification (Hallberg and Palmer, 2013). The chromosomal translocations are the most common genetic alterations in this gene, which result in creation of 22 different fusion genes in different type of tumors, including ALK/NPM (Morris et al., 1994), ALK/EML4 (Soda et al., 2007), ALK/TFG (Rikova et al., 2007), ALK/KIF5B (Takeuchi et al., 2009), ALK/RANBP2, ALK/ATIC, ALK/SQSTM1, ALK/CLTC, ALK/TPM4 and ALK/MSN. An alternative mechanism for ALK activation is through point mutation of the ALK locus, most commonly within the kinase domain, as reported in patients with neuroblastoma, NSCLC and ATC.

ALK mRNA and protein levels diminish in all tissues after birth—they reach their minimum levels at 3 weeks of age and are maintained at low levels in adult animals. ALK is not required for viability in mammals, as Alk^{-/-} mice are viable (Bilsland et al., 2008; Weiss et al., 2012). These physiological function studies of ALK in mammals suggest that pharmacological degradation of ALK using the PROTAC (PROteolysis TArgeting Chimera) or SNIPER (Specific and Nongenetic IAP-dependent Protein ERasers) strategy should be well tolerated in humans. Thus, ALK degraders/disruptors, which refer to bi-functional small molecule compounds that degrade and/or disrupt ALK are expected to display minimal toxicity in the clinic.

Significant effort has been made to develop therapeutics capable of inhibiting the kinase activity of ALK. Four ALK inhibitors have been approved by FDA for the treatment of patients with ALK-positive NSCLC including crizotinib, ceritinib, alectinib and brigatinib (Lin et al., 2017). Various clinical trials are undergoing to investigate potential applications of these drugs in the treatment of other diseases. Besides these FDA approved drugs, a number of ALK inhibitors including lorlatinib, TPX-0005, belizatinib, ensartinib, and CEP-37440 are under clinical investigation.

Clinical results have shown that patients with ALK-positive lung cancer show remarkable responses and increased progression-free survival when treated with ALK inhibitor Crizotinib (Kwak et al., 2010; Solomon et al., 2014), Ceritinib (Shaw and Engelman, 2014) and Alectinib (Liu et al., 2015; Peters et al., 2017). Despite the initial response to such treatments, however, the majority of these patients eventually develop resistance to such treatment within 5 months (Choi et al., 2010; Lin et al., 2017). Preliminary data suggest that such acquired resistance can arise from secondary ALK mutations, gene amplification and also ALK-independent activation that bypass the ALK signaling pathway. Overall, the clinical efficacy of ALK inhibitor monotherapy is limited by the invariable emergence of drug resistance. Hence, developing new therapeutic strategies to overcome or prevent resistance is an urgent priority. Our ALK degraders offer a novel strategy for the treatment of ALK-mediated cancer. Additionally, the nature of our degraders to target ALK for degradation, as opposed to inhibiting the catalytic activity of ALK, would overcome the resistance regardless the drugs that were used in the prior treatment or whether acquired resistance was caused by gene mutation or amplification.

In some aspects, the document provides a method of treating the ALK-positive cancers, the method including administering to a subject in need thereof with an ALK-positive cancer one or more bivalent compounds including an ALK ligand conjugated to a degradation/disruption tag. The ALK-positive cancer may be a cancer resulted from ALK gene fusion, mutation or amplification. The ALK-positive cancer can have elevated ALK enzymatic activity relative to a wild-type tissue of the same species and tissue type. Non-limiting examples of ALK-positive cancer include anaplastic large cell lymphoma; non-Hodgkin's lymphoma; an inflammatory myofibrolastic tumor; a neuroblastoma; sarcoma; lung, non-small cell lung cancer; bronchus; prostate; breast (including sporadic breast cancers and sufferers of Cowden disease); pancreas; gastrointestinal cancer; colon; rectum; colon carcinoma; colorectal adenoma; esophageal cancer, thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary; multiple myeloma; esophagus; a leukemia; acute myelogenous leukemia; chronic myelogenous leukemia; lymphocytic leukemia; lymphoma; myeloid leukemia; brain; a carcinoma of the brain; oral cavity and pharynx; larynx; rhabdomyosarcoma; spitz cancer, small intestine; and melanoma. The ALK-positive cancer can be a relapsed cancer. The ALK-positive cancer can have been refractory to one or more previous treatments by different ALK inhibitors.

### SUMMARY

The present disclosure relates generally to bivalent compounds (e.g., bi-functional small molecule compounds) which degrade and/or disrupt ALK, ALK fusion proteins, and/or ALK mutant proteins, and to methods for the treatment of ALK-mediated cancer (i.e., a cancer which depends on ALK fusion; cancer which depends on ALK mutation; cancer which depends on ALK amplification; cancer which depends on ALK activity; or cancer having elevated levels of ALK, or ALK activity relative to a wild-type tissue of the same species and tissue type). It is important to note, because the ALK degraders/disruptors have dual functions (enzyme inhibition plus protein degradation/disruption), the bivalent compounds disclosed/claimed here can be significantly more effective therapeutic agents than current ALK inhibitors, which inhibit the enzymatic activity of ALK, ALK fusion proteins, and/or ALK mutant proteins, but do not affect ALK protein levels. The present disclosure further provides methods for identifying ALK degraders/disruptors as described herein.

More specifically, the present disclosure provides a bivalent compound including an ALK ligand conjugated to a degradation/disruption tag.

In some aspects, the ALK degraders/disruptors have the form “PI-Linker-EL”, as shown below:

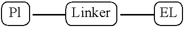

wherein PI comprises an ALK ligand (e.g., an ALK inhibitor) and EL comprises a degradation/disruption tag (e.g., E3 ligase ligand). Exemplary ALK ligands (PI), exemplary degradation/disruption tags (EL), and exemplary linkers (Linker) are illustrated below:
PI includes but is not limited to:

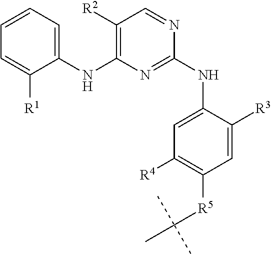

wherein,
R¹ is (CR⁶R⁷)ₙSO₂R⁸, (CR⁶R⁷)ₙSO₂NR⁸R⁹, (CR⁶R⁷)ₙCOR⁸, (CR⁶R⁷)ₙCO₂R⁸, (CR⁶R⁷)ₙCONR⁸R⁹, (CR⁶R⁷)ₙP(O)R⁸R⁹, (CR⁶R⁷)ₙP(O)NR⁸R⁹;
R², R³ and R⁴ are independently hydrogen, halogen, C1-C8 alkyl, C1-C8 alkoxy, C1-C8 alkoxy alkyl;
R⁵ are independently hydrogen, halogen, C1-C8 alkyl, C1-C8 alkoxy, C1-C8 alkoxy alkyl, C1-C8 haloalkyl, C1-C8 hydroxyalkyl, C3-C7 cycloalkyl, C3-C7 heterocyclyl, C2-C8 alkenyl, C2-C8 alkynyl, OR¹⁰, SR¹⁰, NR¹⁰R¹¹, CN, NO₂, (CR¹⁰R¹¹)mNR¹²R¹³, (CR¹⁰R¹¹)mC(O)R¹², (NR¹⁰R¹¹)mNR¹²R¹³, (NR¹⁰R¹¹)mC(O)R¹², COR¹⁰, CO₂R¹⁰, CONR¹⁰R¹¹, NR¹⁰COR¹, NR¹⁰SOR¹¹, NR¹⁰SO₂R¹¹, SOR¹⁰, SO₂R¹⁰, SO₂NR¹⁰R¹¹, (CR¹⁰R¹¹)m-aryl, or (CR¹⁰R¹¹)m-heteroaryl;
m=0-8;
n=0-3;
R⁶, R⁷, R⁸, R⁹, R¹⁰, R¹¹, R¹², R¹³ are independently hydrogen, C1-C8 alkyl, C1-C8 alkoxy, C2-C8 alkenyl, C2-C8 alkynyl, arylalkyl, cycloalkyl, heterocycloalkyl, aryl, heteroaryl, or heteroarylalkyl; and
R⁶ and R⁷, R⁸ and R⁹, R¹⁰ and R¹¹, R¹² and R¹³ can independently form 4-8 membered alkyl or heterocyclyl rings.
The ALK ligand can be an ALK inhibitor, such as, for example, crizotinib, ceritinib, alectinib, brigatinib, lorlatinib, TPX-0005, belizatinib, ensartinib, CEP-37440, and/or analogs thereof.

In some embodiments, the ALK ligand can be, e.g.,

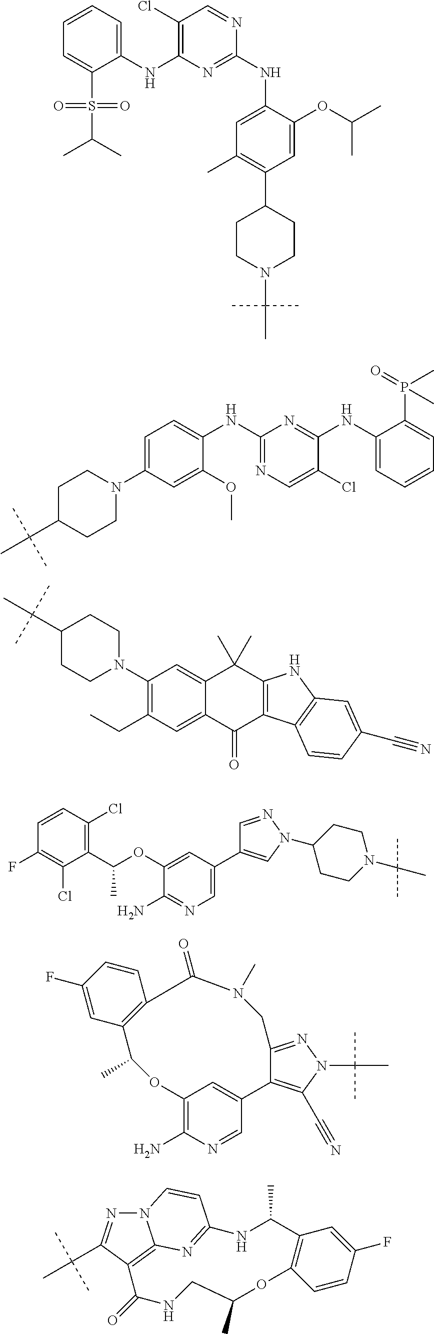

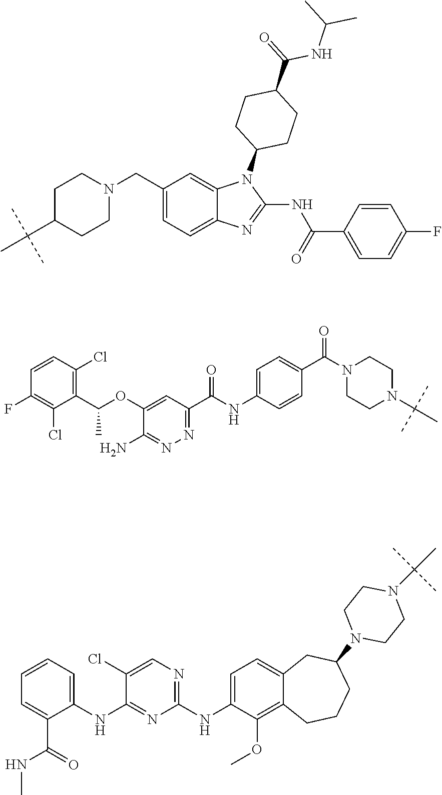

The ALK ligand can be bound to ALK, ALK fusion proteins, and/or ALK mutant proteins.
EL includes but is not limited to:

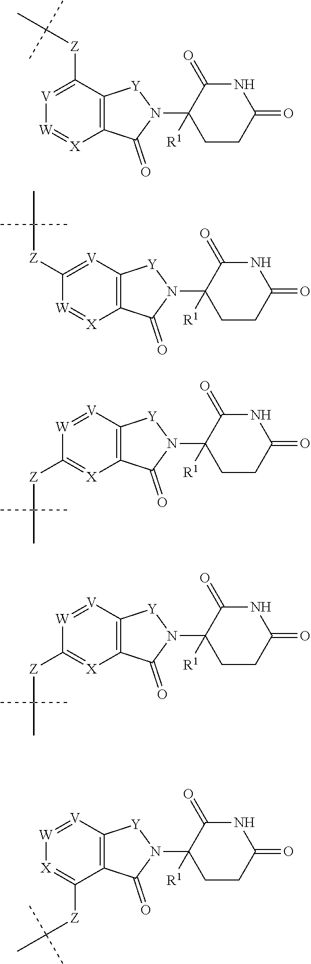

wherein
V, W, X are independently CR², or N;

### Y is CO or CH₂;

### Z is CH₂, NH, or O;

R¹ is hydrogen, methyl, or fluoro; and
R² is hydrogen, halogen, or C1-C5 alkyl;

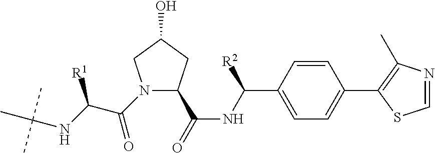

wherein
R¹ and R² are independently hydrogen, C1-C8 alkyl, C1-C8 alkoxyalkyl, C1-C8 haloalkyl, C1-C8 hydroxyalkyl, C3-C7 cycloalkyl, C3-C7 heterocyclyl, C2-C8 alkenyl, or C2-C8 alkynyl; and

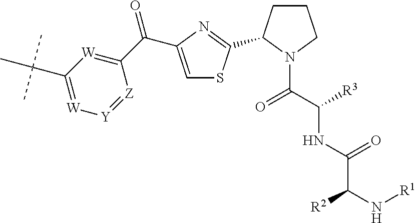

wherein
R¹, R², R³ and R⁴ are independently hydrogen, C1-C8 alkyl, C1-C8 alkoxyalkyl, C1-C8 haloalkyl, C1-C8 hydroxyalkyl, C3-C7 cycloalkyl, C3-C7 heterocyclyl, C2-C8 alkenyl, or C2-C8 alkynyl; and
V, W, X, Z are independently CR⁴, or N.

In some embodiments, the degradation/disruption tag can be for example, pomalidomide, thalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, and/or analogs thereof.

In some embodiments, the degradation/disruption tag can be, e.g.,

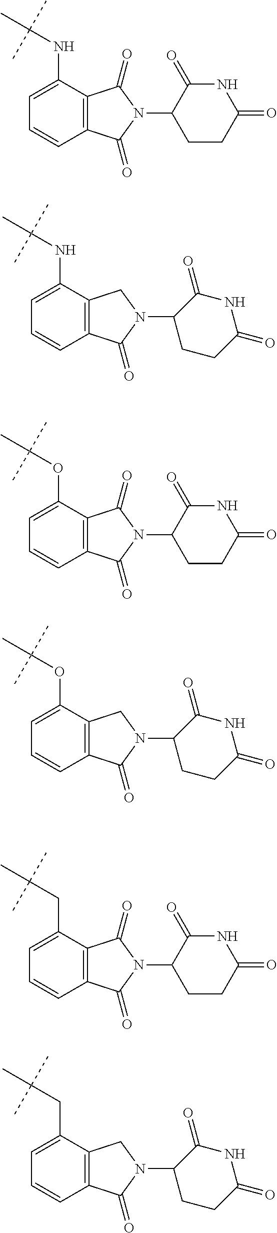

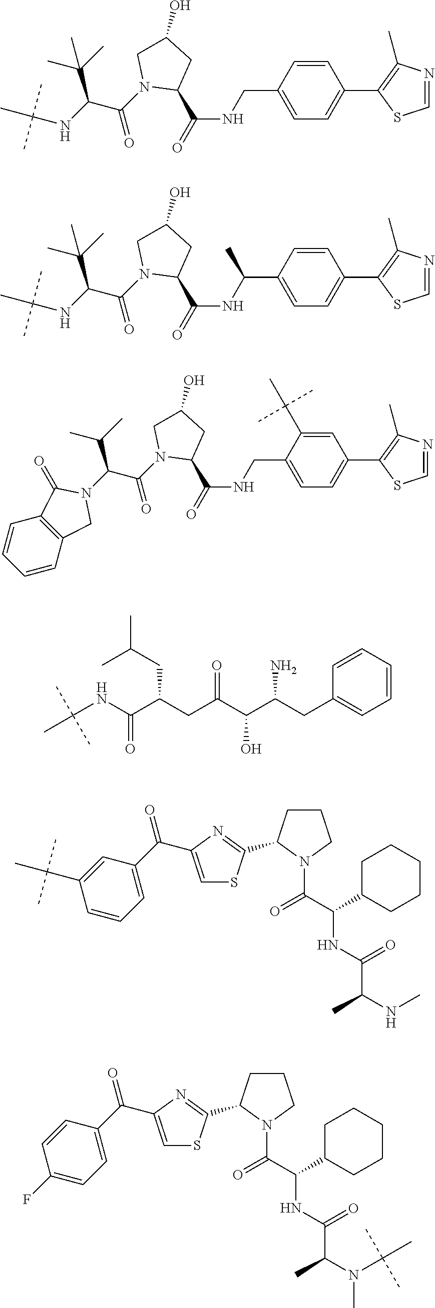

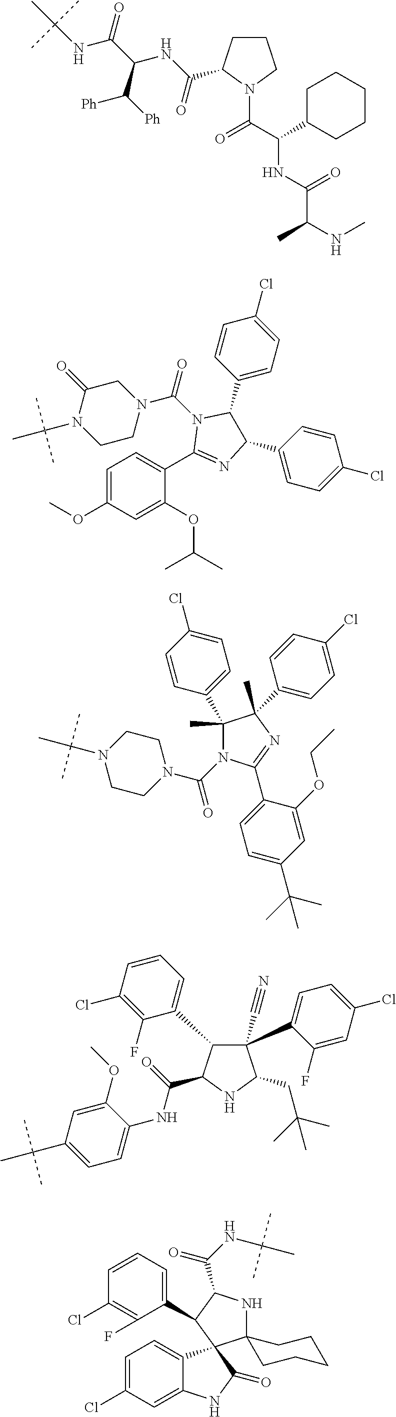

In some embodiments, the degradation/disruption tag can bind to a ubiquitin ligase (e.g., an E3 ligase such as a cereblon E3 ligase, a VHL E3 ligase, a MDM2 ligase, a TRIM21 ligase, a TRIM24 ligase, and/or an IAP ligase) and/or serve as a hydrophobic group that leads to ALK protein misfolding.

In any of the above-described compounds, the ALK ligand can be conjugated to the degradation/disruption tag through a linker. The linker can include, for example, acyclic or cyclic saturated or unsaturated carbon, ethylene glycol, amide, amino, ether, urea, carbamate, aromatic, heteroaromatic, heterocyclic and/or carbonyl containing groups with different lengths.

In some embodiments, the linker can be a moiety of:

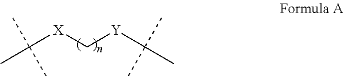

wherein

### X is C═O or CH₂;

### Y is C═O or CH₂; and

n is 0-15.

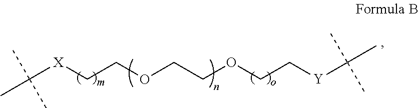

wherein

### X is C═O or CH₂;

### Y is C═O or CH₂;

m is 0-15;
n is 0-6; and
o is 0-15.

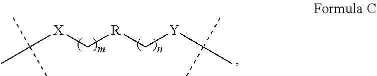

wherein

### X is C═O or CH₂;

### Y is C═O or CH₂;

R is —CH₂—, —CF₂—, —CH(C₁₋₃ alkyl)-, —C(C₁₋₃ alkyl)(C₁₋₃ alkyl), —CH═CH—, —C(C₁₋₃ alkyl)≡C(C₁₋₃ alkyl)-, —C═C—, —O—, —NH—, —N(C₁₋₃ alkyl)-, —C(O)NH—, —C(O)N(C₁₋₃ alkyl)-, a 3-13 membered ring, a 3-13 membered fused ring, a 3-13 membered bridged ring, and/or a 3-13 membered spiro ring;
m is 0-15; and
n is 0-15.

In some embodiments of Formula C, R is a 3-13 membered ring, a 3-13 membered fused ring, a 3-13 membered bridged ring, and/or a 3-13 membered spiro ring, one or more of which can contain one or more heteroatoms.

In some embodiments of Formula C, R has a structure of

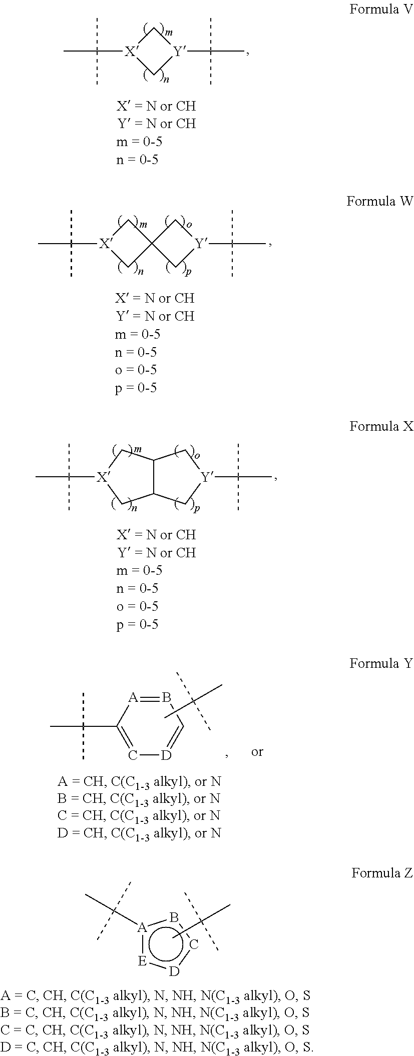

In some embodiments, the bivalent compound is a compound selected from CZ40-50, CZ40-51, CZ40-52, CZ40-53, CZ40-77, CZ40-78, CZ40-79, CZ40-80, CZ40-81, CZ40-82, CZ40-83, CZ40-84, CZ40-85, CZ40-86, CZ40-87, CZ40-88, CZ40-89, CZ40-90, CZ40-92, CZ40-93, CZ40-94, CZ47-01, CZ47-02, CZ47-03, CZ47-04, CZ47-05, CZ47-06, CZ47-07, CZ47-08. CZ47-09, CZ47-10, CZ47-11, CZ47-12, CZ47-13, CZ47-14, CZ47-15, CZ47-16, CZ47-17, CZ47-18, CZ47-19, CZ47-20, CZ47-21, CZ47-22, CZ47-23, CZ47-24, CZ47-25, CZ47-26, CZ47-27, CZ47-28, CZ47-29, CZ47-40, CZ47-48, HC58-98, HC58-99, HC58-100, HC58-110, HC58-111, HC58-112, or analogs thereof.

In some aspects, the document provides a method of treating the ALK-positive cancers, the method including administering to a subject in need thereof with an ALK-positive cancer one or more bivalent compounds including an ALK ligand conjugated to a degradation/disruption tag. The ALK-positive cancer may be a cancer resulted from ALK gene fusion, mutation or amplification. The ALK-positive cancer can have elevated ALK enzymatic activity relative to a wild-type tissue of the same species and tissue type. Non-limiting examples of ALK-positive cancer include anaplastic large cell lymphoma; non-Hodgkin's lymphoma; an inflammatory myofibrolastic tumor; a neuroblastoma; sarcoma; lung, non-small cell lung cancer; bronchus; prostate; breast (including sporadic breast cancers and sufferers of Cowden disease); pancreas; gastrointestinal cancer; colon; rectum; colon carcinoma; colorectal adenoma; esophageal cancer, thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary; multiple myeloma; esophagus; a leukemia; acute myelogenous leukemia; chronic myelogenous leukemia; lymphocytic leukemia; lymphoma; myeloid leukemia; brain; a carcinoma of the brain; oral cavity and pharynx; larynx; rhabdomyosarcoma; spitz cancer, small intestine; and melanoma. The ALK-positive cancer can be a relapsed cancer. The ALK-positive cancer can have been refractory to one or more previous treatments by different ALK inhibitors.

In any of the above-described methods, the bivalent compounds can be CZ40-50, CZ40-51, CZ40-52, CZ40-53, CZ40-77, CZ40-78, CZ40-79, CZ40-80, CZ40-81, CZ40-82, CZ40-83, CZ40-84, CZ40-85, CZ40-86, CZ40-87, CZ40-88, CZ40-89, CZ40-90, CZ40-92, CZ40-93, CZ40-94, CZ47-01, CZ47-02, CZ47-03, CZ47-04, CZ47-05, CZ47-06, CZ47-07, CZ47-08. CZ47-09, CZ47-10, CZ47-11, CZ47-12, CZ47-13, CZ47-14, CZ47-15, CZ47-16, CZ47-17, CZ47-18, CZ47-19, CZ47-20, CZ47-21, CZ47-22, CZ47-23, CZ47-24, CZ47-25, CZ47-26, CZ47-27, CZ47-28, CZ47-29, CZ47-40, CZ47-48, HC58-98, HC58-99, HC58-100, HC58-110, HC58-111, HC58-112, or analogs thereof.

In some embodiments of the disclosed methods, the bivalent compounds can be administered, e.g., orally, parenterally, intradermally, subcutaneously, topically, and/or rectally.

Any of the above-described methods can further include treating the subject with one or more additional therapeutic regimens for treating cancer. The one or more additional therapeutic regimens for treating cancer can be, e.g., one or more of surgery, chemotherapy, radiation therapy, hormone therapy, or immunotherapy.

The document additionally provides a method for identifying a bivalent compound which mediates degradation/disruption of ALK, the method including providing a heterobifunctional test compound including a ALK ligand conjugated to a degradation/disruption tag, contacting the heterobifunctional test compound with a cell (e.g., a cancer cell such as a ALK-mediated cancer cell) which contains a ubiquitin ligase and ALK.

As used herein, the terms “about” and “approximately” are defined as being within plus or minus 10% of a given value or state, preferably within plus or minus 5% of said value or state.

Unless otherwise defined, all technical and scientific terms used herein have the same meaning as commonly understood by one of ordinary skill in the art to which this invention belongs.

Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a series of Western blots showing the effect of various ALK degraders in reducing NPM-ALK fusion protein expression at the 100 nM concentration in SU-DHL-1 cells.

FIG. 2 is a series of Western blots showing the effect of various ALK degraders in reducing NPM-ALK fusion protein expression at the 30 nM concentration in SU-DHL-1 cells.

FIG. 3 is a series of Western blots showing the effect of two ALK degraders in reducing NPM-ALK fusion protein expression in SU-DHL-1 cells in a concentration-dependent manner.

FIG. 4 is a series of Western blots showing the effect of two ALK degraders in reducing EML4-ALK fusion protein concentration-dependently in NCI-H2228 non-small cell lung adenocarcinoma cancer cells.

FIG. 5 is a series of cell viability assay results showing the effect of three ALK degraders in inhibiting SU-DHL-1 cancer cell growth.

FIG. 6 is a series of Western blots showing the effect of two ALK degraders on reducing ALK fusion protein levels and inhibiting the ALK down-stream signaling in a concentration-dependent manner in SU-DHL-1 cells.

FIG. 7 is a series of Western blots showing the effect of two ALK degraders on reducing ALK fusion protein levels and inhibiting the ALK down-stream signaling in a concentration-dependent manner in NCI-H2228 cells.

FIG. 8 is a series of Western blots showing the effect of two ALK degraders on reducing ALK fusion protein levels and inhibiting the ALK down-stream signaling in a time-dependent manner in SU-DHL-1 cells.

FIG. 9 is a series of Western blots showing the effect of two ALK degraders on reducing ALK fusion protein levels and inhibiting the ALK down-stream signaling in a time-dependent manner in NCI-H2228 cells.

FIG. 10 is a series of Western blots showing that the effect of two ALK degraders on reducing ALK fusion protein levels can be rescued by competitive compounds in SU-DHL-1 cells.

FIG. 11 is a series of Western blots showing that the effect of two ALK degraders on reducing ALK fusion protein levels can be recovered after media washout in SU-DHL-1 cells.

FIG. 12 is a figure showing the effect of ceritinib. two ALK degraders, and two negative control compounds on inhibiting the cell viability of SU-DHL-1 cells.

FIG. 13 is a series of Western blots showing the effect of six ALK degraders on reducing ALK fusion protein levels in a concentration-dependent manner in NCI-H3122 cells.

FIG. 14 is a series of Western blots showing the effect of six ALK degraders on reducing ALK fusion protein levels in a concentration-dependent manner in SU-DHL-1 cells.

FIG. 15 is a series of Western blots showing the effect of three ALK degraders and three negative control compounds on reducing ALK fusion protein levels in SU-DHL-1 and NCI-H3122 cells.

FIG. 16 is a series of Western blots showing that the effect of three ALK degraders on reducing ALK fusion protein levels can be rescued by competitive compounds in SU-DHL-1 cells.

FIG. 17 is a figure showing the effect of ceritinib and six ALK degraders on inhibiting the cell viability of SU-DHL-1 and NCI-H3122 cells.

FIG. 18 is a figure showing that CZ40-78 is bioavailable in mice.

### DETAILED DESCRIPTION

The present disclosure is based, in part, on the discovery that novel heterobifunctional small molecules which degrade ALK, ALK fusion proteins, and/or ALK mutant proteins (“PROteolysis TArgeting Chimeras” or “PROTACs”; “Specific and Nongenetic IAP-dependent Protein Erasers” or “SNIPER”) are useful in the treatment of ALK-mediated cancers, particularly neuroblastoma, esophageal cancers, colorectal cancers, breast cancers, renal cancers, thyroid cancers, rhabdomyosarcoma, myofibroblastic cancers, spitz cancers, lymphoma and lung cancers.

Successful strategies for selective degradation/disruption of the target protein induced by a small molecule include recruiting an E3 ubiquitin ligase and mimicking protein misfolding with a hydrophobic tag (Buckley and Crews, 2014). PROTACs are bivalent inhibitors with one moiety that binds an E3 ubiquitin ligase and another moiety that binds the protein target of interest (Buckley and Crews, 2014). The induced proximity leads to ubiquitination of the target followed by their degradation at proteasome. Two types of high affinity small-molecule E3 ligase ligands have been identified/developed: immunomodulatory drugs (IMiDs) such as thalidomide and pomalidomide, which bind cereblon (CRBN or CRL4CRBN), a component of a cullin-RING ubiquitin ligase (CRL) complex (Bondeson et al., 2015; Chamberlain et al., 2014; Fischer et al., 2014; Ito et al., 2010; Winter et al., 2015); and VHL-1, a hydroxyproline-containing ligand, which binds van Hippel-Lindau protein (VHL or CRL2VHL), a component of another CRL complex (Bondeson et al., 2015; Buckley et al., 2012a; Buckley et al., 2012b; Galdeano et al., 2014; Zengerle et al., 2015). The PROTAC technology has been successfully applied to degradation of multiple targets (Bondeson et al., 2015; Buckley et al., 2015; Lai et al., 2016; Lu et al., 2015; Winter et al., 2015; Zengerle et al., 2015), but not to degradation of ALK, ALK fusion proteins, or ALK mutant proteins. In addition, a hydrophobic tagging approach, which utilizes a bulky and hydrophobic adamantyl group, has been developed to mimic protein misfolding, leading to the degradation of the target protein by proteasome (Buckley and Crews, 2014). This approach has also been successfully applied to selective degradation of the pseudokinase Her3 (Xie et al., 2014), but not to degradation of ALK, ALK fusion proteins, or ALK mutant proteins.

As discussed in the following examples, this disclosure provides specific examples of novel ALK degraders/disruptors, and examined the effect of exemplary degraders/disruptors in inhibiting/disrupting ALK activity, suppressing ALK expression, and inhibiting cancer cell proliferation. The results indicated that these novel small molecules can be beneficial in treating cancer, especially ALK-positive non-small cell lung cancer (NS CL), ALK-positive Anaplastic Large Cell Lymphoma (ALCL) and other tumors with aberrations in ALK.

A number of selective small-molecule ALK catalytic inhibitors, such as crizotinib, ceritinib, alectinib, brigatinib, lorlatinib, TPX-0005, belizatinib, ensartinib, and CEP-37440 have recently been discovered. Some of these inhibitors have been in clinical trials for treating non-small-cell lung cancer (NSCLC) and anaplastic large cell lymphoma (ALCL). Despite the initial response to such treatments, however, the majority of these patients eventually develop resistance to such treatment within 5 months (Choi et al., 2010; Lin et al., 2017). Preliminary data suggest that such acquired resistance can arise from secondary ALK mutations, gene amplification and also ALK-independent activation that bypass the ALK signaling pathway. Overall, the clinical efficacy of ALK inhibitor monotherapy is limited by the invariable emergence of drug resistance.

Current drugs targeting ALK generally focus on inhibition of its catalytic function. Here, a different approach was taken: to develop compounds that directly and selectively target not only the catalytic function of ALK, but also their level of expression at the protein level. Strategies for inducing protein degradation include recruiting E3 ubiquitin ligases, mimicking protein misfolding with hydrophobic tags, and inhibiting chaperones. For example, a thalidomide-JQ1 bivalent compound has been used to hijack the cereblon E3 ligase, inducing highly selective BET protein degradation in vitro and in vivo and resulting in a demonstrated delay in leukemia progression in mice (Winter et al., 2015). Similarly, BET protein degradation has also been induced via another E3 ligase, VHL (Zengerle et al., 2015). Partial degradation of Her3 has been induced using an adamantane-modified compound (Xie et al., 2014). Such an approach, based on the use of bivalent small molecule compounds, permits more flexible regulation of protein expression in vitro and in vivo compared with techniques such as gene knockout or shRNA knockdown. Unlike gene knockout or shRNA knockdown, a small molecule approach further provides an opportunity to study dose and time dependency in a disease model through varying the concentrations and frequencies of administration of the relevant small molecule.

### PROTACs and SNIPERs

In some aspects, the present disclosure provides bivalent compounds, also referred to herein as PROTACs and SNIPERs, comprising an ALK ligand (or targeting moiety) conjugated to a degradation tag. Linkage of the ALK ligand to the degradation tag can be direct, or indirect via a linker.

As used herein, the terms “Anaplastic lymphoma kinase (ALK) ligand” or “ALK ligand” or “ALK targeting moiety” are to be construed broadly, and encompass a wide variety of molecules ranging from small molecules to large proteins that associates with or binds to ALK. The ALK ligand or targeting moiety can be, for example, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof.

The ALK ligand or targeting moiety can be an ALK inhibitor (e.g., crizotinib, ceritinib, alectinib, brigatinib, lorlatinib, TPX-0005, belizatinib, ensartinib, CEP-37440, and analogs thereof) which is capable of interfering with the enzymatic activity of ALK. As used herein, an “inhibitor” refers to an agent that restrains, retards, or otherwise causes inhibition of a physiological, chemical or enzymatic action or function. An inhibitor can cause an at least 5% decrease in enzyme activity. An inhibitor can also or alternately refer to a drug, compound, or agent that prevents or reduces the expression, transcription, or translation of a gene or protein. An inhibitor can reduce or prevent the function of a protein, e.g., by binding to or activating/inactivating another protein or receptor.

Exemplary ALK ligands include, but are not limited to, the compounds listed below.

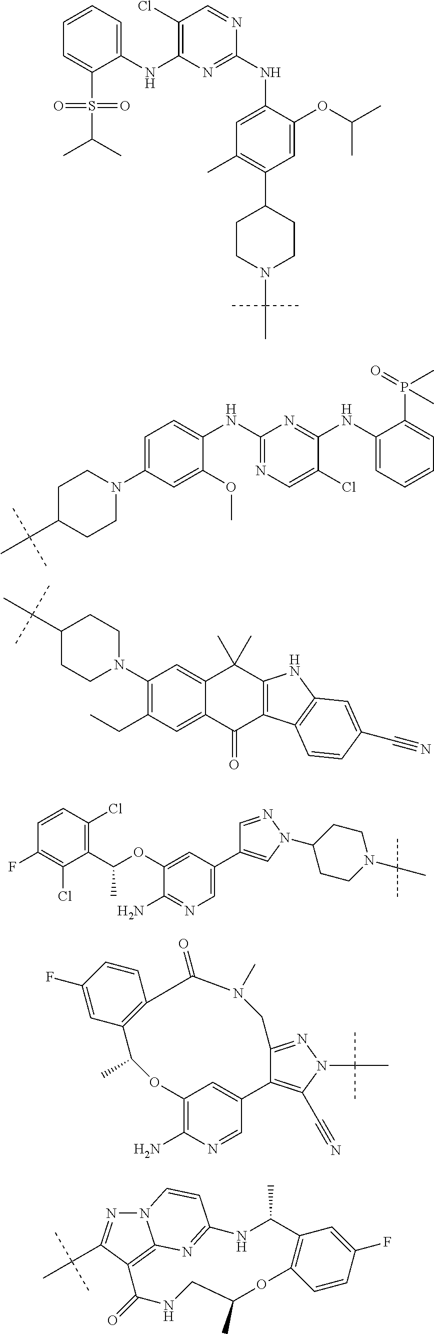

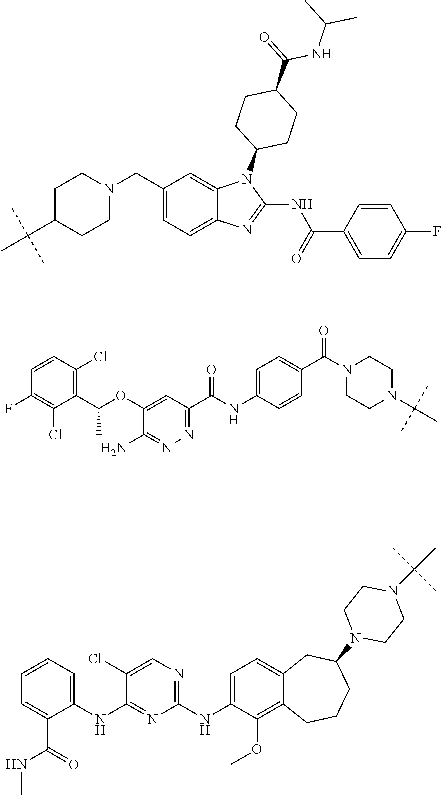

As used herein, the term “degradation/disruption tag” refers to a compound which associates with or binds to a ubiquitin ligase for recruitment of the corresponding ubiquitination machinery to ALK or induces ALK protein misfolding and subsequent degradation at the proteasome or loss of function.

In some aspects, the degradation/disruption tags of the present disclosure include, e.g., thalidomide, pomalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, and/or analogs thereof.

As used herein, a “linker” is a bond, molecule, or group of molecules that binds two separate entities to one another. Linkers can provide for optimal spacing of the two entities. The term “linker” in some aspects refers to any agent or molecule that bridges the ALK ligand to the degradation/disruption tag. One of ordinary skill in the art recognizes that sites on the ALK ligand or the degradation/disruption tag, which are not necessary for the function of the PROTACs or SNIPERs of the present disclosure, are ideal sites for attaching a linker, provided that the linker, once attached to the conjugate of the present disclosures, does not interfere with the function of the PROTAC or SNIPER, i.e., its ability to target ALK and recruit a ubiquitin ligase.

The length of the linker of the bivalent compound can be adjusted to minimize the molecular weight of the disruptors/degraders and avoid the clash of the ALK ligand or targeting moiety with the ubiquitin ligase or induce ALK misfolding by the hydrophobic tag at the same time.

As used herein, the term “analog” is used in accordance with its plain ordinary meaning within chemistry and biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound.

In some embodiments, the degradation/disruption tags of the present disclosure include, for example, thalidomide, pomalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, and analogs thereof. The degradation/disruption tags can be attached to each portion of interest in the structure of a ALK ligand or targeting moiety (e.g., crizotinib, ceritinib, alectinib, brigatinib, lorlatinib, TPX-0005, belizatinib, ensartinib, and CEP-37440) with linkers of different types and lengths in order to generate effective bivalent compounds. In particular, attaching pomalidomide to either portion of the molecule can recruit the cereblon E3 ligase to ALK.

The bivalent compounds disclosed herein can selectively affect ALK-mediated cancer cells compared to WT cells (i.e., an ALK degrader/disruptor able to kill or inhibit the growth of an ALK-mediated cancer cell while also having a relatively low ability to lyse or inhibit the growth of a WT cell), e.g., possess a GI₅₀ for one or more ALK-mediated cancer cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the ALK-mediated cancer cells.

Additional bivalent compounds (i.e., ALK degraders/disruptors) can be developed using the principles and methods disclosed herein. For example, other linkers, degradation tags, and ALK binding/inhibiting moieties (not limited to crizotinib, ceritinib, alectinib, brigatinib, lorlatinib, TPX-0005, belizatinib, ensartinib, and CEP-37440) can be synthesized and tested.

Non-limiting examples of ALK disruptors/degraders (e.g., bivalent compounds) are shown in Table 1 (below). The left portion of the ALK disruptors/degraders bind to ALK (as crizotinib, ceritinib, alectinib, brigatinib, lorlatinib, TPX-0005, belizatinib, ensartinib, and CEP-37440 do), and the right portion recruits for the ubiquitination machinery to ALK, which induces the poly-ubiquitination and degradation of ALK at the proteasome.

Non-limiting examples of bivalent compounds are set forth in Table 1, below.

TABLE 1

| Cpd. | | | |
|---|---|---|---|
| Ex- | Cpd. | | |
| ample | | | |
| Number | Code | Structure | Chemical Name |
| 1 | CZ40-50 | 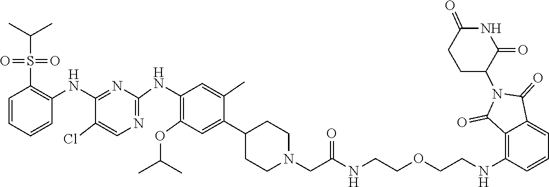 [SMILES: Cc1cc(Nc2ncc(Cl)c(Nc3ccccc3S(=O)(=O)C(C)C)n2)c(OC(C)C)cc1C1CCN(CC(=O)NCCOCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)- N-(2-(2-((2-(2,6- dioxopiperidin-3- yl)-1,3- |
| | | | dioxoisoindolin- |
| | | | 4-yl) |
| | | | amino)ethoxy) |
| | | | ethyl)acetamide |
| 2 | CZ40-51 | 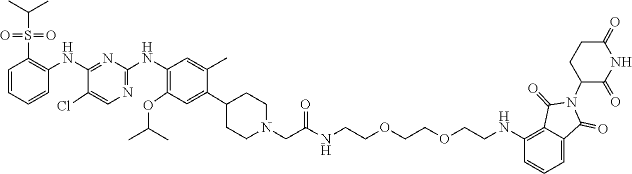 [SMILES: Cc1cc(Nc2ncc(Cl)c(Nc3ccccc3S(=O)(=O)C(C)C)n2)c(OC(C)C)cc1C1CCN(CC(=O)NCCOCCOCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)- N-(2-(2-(2-((2-(2, 6-dioxopiperidin- |
| | | | 3-yl)-1,3- |
| | | | dioxoisoindolin- |
| | | | 4-yl)amino) |
| | | | ethyoxy)ethoxy) |
| | | | ethyl)acetamide |
| 3 | CZ40-52 | 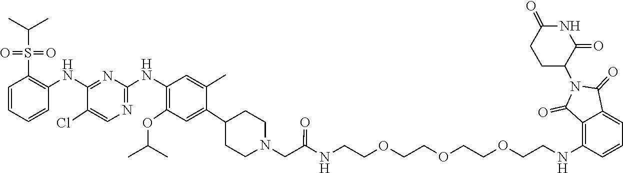 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (2-(2-(2-(2-((2-(2, 6-dioxopiperidin- |
| | | | 3-yl)-1,3- |
| | | | dioxoisoindolin- |
| | | | 4-yl)amino)ethoxy) |
| | | | ethyoxy)ethoxy) |
| | | | ethyl)acetamide |
| 4 | CZ40-53 | 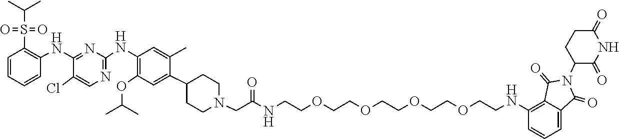 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- |
| | | | (14-((2-(2,6- |
| | | | dioxopiperidin- |
| | | | 3-yl)-1,3- |
| | | | dioxoisoindolin-4- |
| | | | yl)amino)-3,6,9,12- |
| | | | tetraoxatetradecyl) |
| | | | acetamide |
| 5 | CZ40-77 | 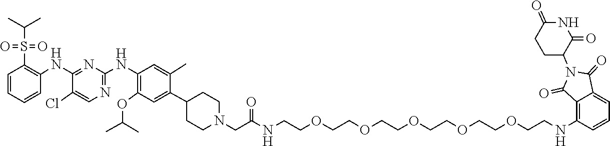 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCOCCNc2cccc3c2C(=O)N(C2CCC(=O)NC2=O)C3=O)CC1] | (2S,4R)-1-((S)- 14-(4-(6-((6- acetyl-8- cyclopentyl-5- methyl-7-oxo-7,8- dihydropyrido [2,3-d]pyrimidin- 2-yl)amino) pyridin-3-yl) |
| | | | piperazin-1-yl)-2- |
| | | | (tert-butyl)-4,14- |
| | | | dioxo-6,9,12- |
| | | | trioxa-3- |
| | | | azatetradecanoyl)- |
| | | | 4-hydroxy-N- |
| | | | (4-(4- |
| | | | methylthiazol-5- |
| | | | yl)benzyl) |
| | | | pyrrolidine- |
| | | | 2-carboxamide |
| 6 | CZ40-78 | 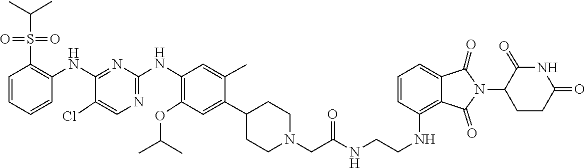 [SMILES: Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (2-((2-(2,6- dioxopiperidin-3- |
| | | | yl)-1,3- |
| | | | dioxoisoindolin-4- |
| | | | yl)amino)ethoxy) |
| | | | acetamide |
| 7 | CZ40-79 | 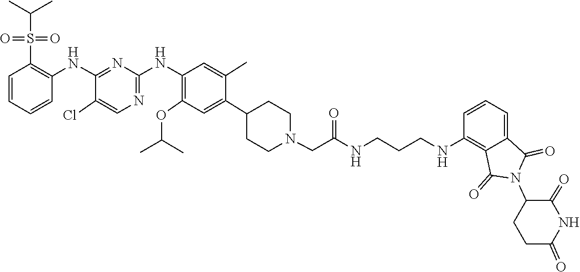 [SMILES: Cc1cc(Nc2ncc(Cl)c(Nc3ccccc3S(=O)(=O)C(C)C)n2)c(OC(C)C)cc1C1CCN(CC(=O)NCCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (2-((3-(2,6- dioxopiperidin-3- yl)-1,3- dioxoisoindolin-4- yl)amino)propyl) acetamide |
| 8 | CZ40-80 | 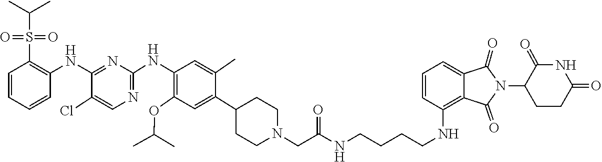 [SMILES: Cc1cc(Nc2ncc(Cl)c(Nc3ccccc3S(=O)(=O)C(C)C)n2)c(OC(C)C)cc1C1CCN(CC(=O)NCCCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (4-((2-(2,6- |
| | | | dioxopiperidin-3- |
| | | | yl)-1,3- |
| | | | dioxoisoindolin-4- |
| | | | yl)amino)butyl) |
| | | | acetamide |
| 9 | CZ40-81 | 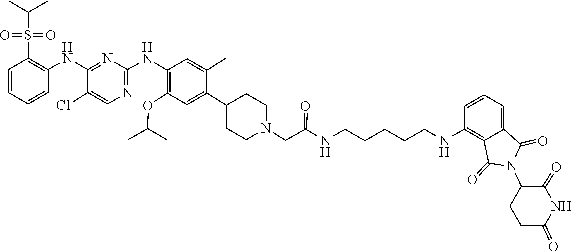 [SMILES: Cc1cc(Nc2ncc(Cl)c(Nc3ccccc3S(=O)(=O)C(C)C)n2)c(OC(C)C)cc1C1CCN(CC(=O)NCCCCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (5-((2-(2,6- dioxopiperidin-3- yl)-1,3- dioxoisoindolin-4- yl)amino)pentyl) acetamide |
| 10 | CZ40-82 | 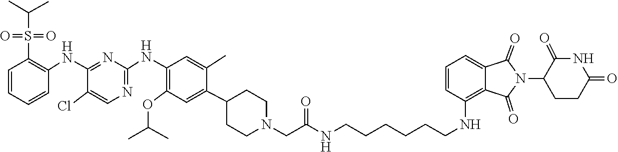 [SMILES: Cc1cc(Nc2ncc(Cl)c(Nc3ccccc3S(=O)(=O)C(C)C)n2)c(OC(C)C)cc1C1CCN(CC(=O)NCCCCCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (6-((2-(2,6- |
| | | | dioxopiperidin-3- |
| | | | yl)-1,3- |
| | | | dioxoisoindolin-4- |
| | | | yl)amino)hexyl) |
| | | | acetamide |
| 11 | CZ40-83 | 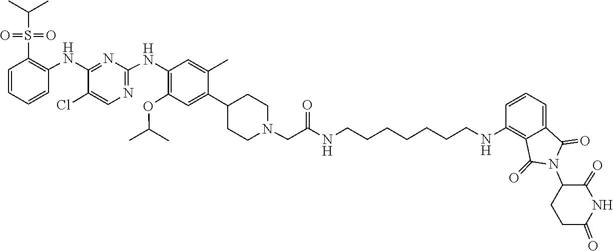 [SMILES: Cc1cc(Nc2ncc(Cl)c(Nc3ccccc3S(=O)(=O)C(C)C)n2)c(OC(C)C)cc1C1CCN(CC(=O)NCCCCCCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (7-((2-(2,6- dioxopiperidin-3- yl)-1,3- dioxoisoindolin-4- yl)amino)heptyl) acetamide |
| 12 | CZ40-84 | 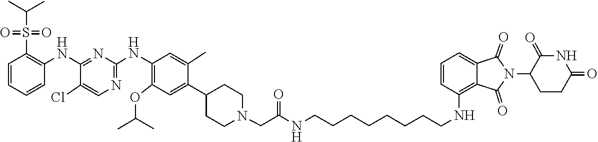 [SMILES: Cc1cc(Nc2ncc(Cl)c(Nc3ccccc3S(=O)(=O)C(C)C)n2)c(OC(C)C)cc1C1CCN(CC(=O)NCCCCCCCCNc2cccc3c2C(=O)N(C2CCC(=O)NC2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- |
| | | | (8-((2-(2,6- |
| | | | dioxopiperidin-3- |
| | | | yl)-1,3- |
| | | | dioxoisoindolin-4- |
| | | | yl)amino)octyl) |
| | | | acetamide |
| 13 | CZ40-85 | 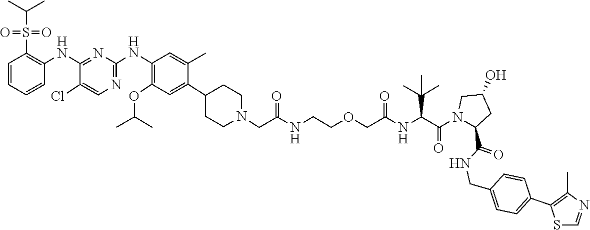 [SMILES: Cc1cc(Nc2ncc(Cl)c(Nc3ccccc3S(=O)(=O)C(C)C)n2)c(OC(C)C)cc1C1CCN(CC(=O)NCCOCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (2-(2-(2-(4-(4-((5- ((5-Chloro-4-((2- (isopropylsulfonyl) phenyl)amino) pyrimidin-2-yl) amino)-5- isopropoxy-2- methylphenyl) piperidin-1-yl)- acetamido)ethoxy) acetamido)-3,3- dimethylbutanoyl)- 4-hydroxy-N-(4-(4- |
| | | | methylthiazol-5- |
| | | | yl)benzyl) |
| | | | pyrrolidine-2- |
| | | | carboxamide |
| 14 | CZ40-86 | 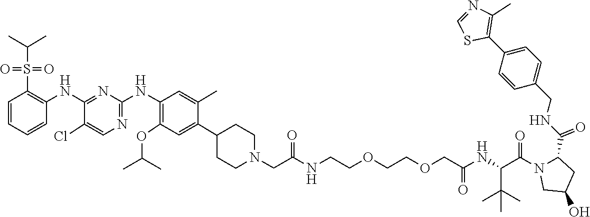 [SMILES: Cc1cc(Nc2ncc(Cl)c(Nc3ccccc3S(=O)(=O)C(C)C)n2)c(OC(C)C)cc1C1CCN(CC(=O)NCCOCCOCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (tert-Butyl)-14-(4- (4-((5-chloro-4- ((2- (isopropylsulfonyl) phenyl)amino) pyrimidin-2-yl) amino)-5- isopropoxy-2- methylphenyl) piperidin-1-yl)- 4,13-dioxo-6,9- dioxa-3,12- diazatetradecanoyl)- |
| | | | 4--hydroxy-N-(4-(4- |
| | | | methylthiazol-5- |
| | | | yl)benzyl) |
| | | | pyrrolidine-2- |
| | | | carboxamide |
| 15 | CZ40-87 | 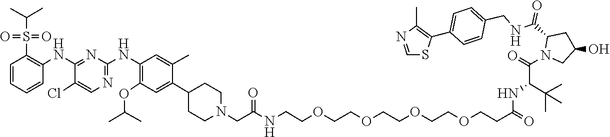 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCC(=O)N[C@H](C(=O)N2C[C@H](O)C[C@H]2C(=O)NCc2ccc(-c3scnc3C)cc2)C(C)(C)C)CC1] | (2S,4R)-1-((S)-20- (tert-Butyl)-1-(4-(4- ((5-chloro-4-((2- (isopropylsulfonyl) phenyl)amino) pyrimidin-2-yl) amino)-5- isopropoxy-2- methylphenyl) |
| | | | piperidin-1-yl)- |
| | | | 2,18-dioxo-6,912, |
| | | | 15-tetraoxa-3,19- |
| | | | diazahenicosan-21- |
| | | | oyl)-4-hydroxy-N- |
| | | | (4-(4- |
| | | | methylthiazol-5- |
| | | | yl)benzyl) |
| | | | pyrrolidine-2- |
| | | | carboxamide |
| 16 | CZ40-88 | 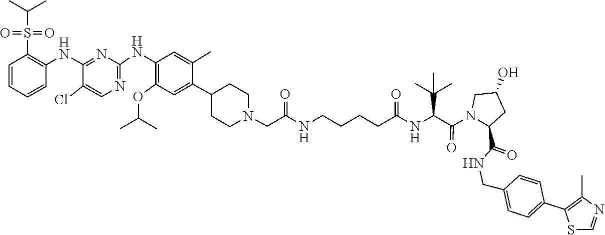 [SMILES: Cc1cc(Nc2ncc(Cl)c(Nc3ccccc3S(=O)(=O)C(C)C)n2)c(OC(C)C)cc1C1CCN(CC(=O)NCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (5-(2-(4-(4-((5- Chloro-4-((2- (isopropylsulfonyl) phenyl)amino) pyrimidin-2-yl) amino)-5- isopropoxy-2- methylphenyl) piperidin-1-yl)- acetamido) pentanamido)-3,3- dimethylbutanoyl)- 4-hydroxy-N-(4-(4- methylthiazol-5- |
| | | | yl)benzyl) |
| | | | pyrrolidine-2- |
| | | | carboxamide |
| 17 | CZ40-89 | 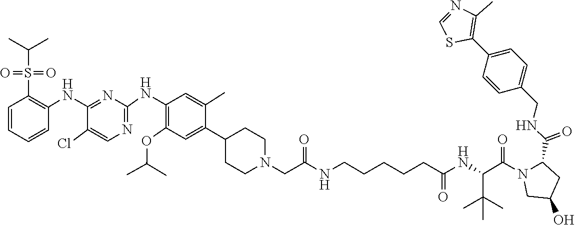 [SMILES: Cc1cc(Nc2ncc(Cl)c(Nc3ccccc3S(=O)(=O)C(C)C)n2)c(OC(C)C)cc1C1CCN(CC(=O)NCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (6-(2-(4-(4-((5- Chloro-4-((2- (isopropylsulfonyl) phenyl)amino) pyrimidin-2-yl) amino)-5- isopropoxy-2- methylphenyl) piperidin-1-yl)- acetamido) hexanamido)-3,3- dimethylbutanoyl)- 4-hydroxy-N-(4-(4- |
| | | | methylthiazol-5- |
| | | | yl)benzyl) |
| | | | pyrrolidine-2- |
| | | | carboxamide |
| 18 | CZ40-90 | 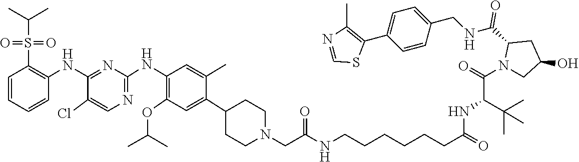 [SMILES: Cc1cc(Nc2ncc(Cl)c(Nc3ccccc3S(=O)(=O)C(C)C)n2)c(OC(C)C)cc1C1CCN(CC(=O)NCCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (7-(2-(4-(4-((5- Chloro-4-((2- (isopropylsulfonyl) phenyl)amino) pyrimidin-2-yl) amino)-5- isopropoxy-2- methylphenyl) piperidin-1-yl)- acetamido) |
| | | | heptanamido)-3,3- |
| | | | dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 19 | CZ40-92 | 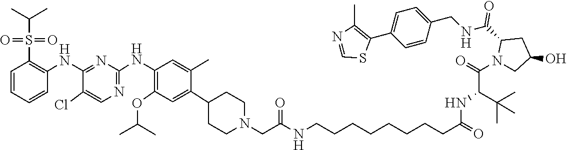 [SMILES: Cc1cc(Nc2ncc(Cl)c(Nc3ccccc3S(=O)(=O)C(C)C)n2)c(OC(C)C)cc1C1CCN(CC(=O)NCCCCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (9-(2-(4-(4-((5- Chloro-4-((2- (isopropylsulfonyl) phenyl)amino) pyrimidin-2-yl) amino)-5- isopropoxy-2- methylphenyl) piperidin-1-yl)- |
| | | | acetamido) |
| | | | nonanamido)-3,3- |
| | | | dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 20 | CZ40-93 | 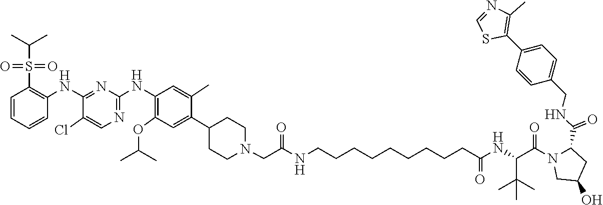 [SMILES: Cc1cc(Nc2ncc(Cl)c(Nc3ccccc3S(=O)(=O)C(C)C)n2)c(OC(C)C)cc1C1CCN(CC(=O)NCCCCCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (10-(2-(4-(4-((5- Chloro-4-((2- (isopropylsulfonyl) phenyl)amino) pyrimidin-2-yl) amino)-5- isopropoxy-2- methylphenyl) piperidin-1-yl)- acetamido) decanamido)-3,3- dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 21 | CZ40-94 | 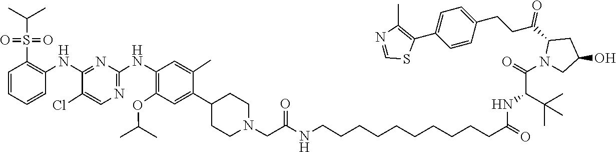 [SMILES: Cc1cc(Nc2ncc(Cl)c(Nc3ccccc3S(=O)(=O)C(C)C)n2)c(OC(C)C)cc1C1CCN(CC(=O)NCCCCCCCCCCC(=O)N[C@H](C(=O)N2C[C@H](O)C[C@H]2C(=O)CCc2ccc(-c3scnc3C)cc2)C(C)(C)C)CC1] | (2S,4R)-1-((S)-2- (10-(2-(4-(4-((5- Chloro-4-((2- (isopropylsulfonyl) phenyl)amino) pyrimidin-2-yl) amino)-5- isopropoxy-2- methylphenyl) piperidin-1-yl)- |
| | | | acetamido) |
| | | | undecanamido)-3,3- |
| | | | dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 22 | CZ47-01 | 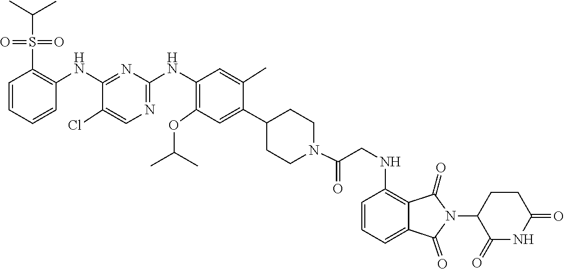 [SMILES: Cc1cc(Nc2ncc(Cl)c(Nc3ccccc3S(=O)(=O)C(C)C)n2)c(OC(C)C)cc1C1CCN(C(=O)CNc2cccc3c2C(=O)N(C2CCC(=O)NC2=O)C3=O)CC1] | 4-((2-(4-(4-((5- Chloro-4-((2- (isopropylsulfonyl) phenyl)amino) pyrimidin-2-yl) amino)-5- isopropoxy-2- methylphenyl) piperidin-1-yl)- 2-oxoethyl) amino)-(2-(2,6- dioxopiperidin-3- yl)-isoindoline- 1,3-dione |
| 23 | CZ47-02 | 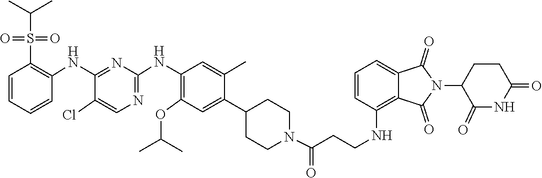 [SMILES: Cc1cc(Nc2ncc(Cl)c(Nc3ccccc3S(=O)(=O)C(C)C)n2)c(OC(C)C)cc1C1CCN(C(=O)CCNc2cccc3c2C(=O)N(C2CCC(=O)NC2=O)C3=O)CC1] | 4-((3-(4-(4-((5- Chloro-4-((2- (isopropylsulfonyl) phenyl)amino) pyrimidin-2-yl) amino)-5- isopropoxy-2- methylphenyl) piperidin-1-yl)- 3-oxopropyl) amino)-(2-(2,6- |
| | | | dioxopiperidin-3- |
| | | | yl)-isoindoline- |
| | | | 1,3-dione |
| 24 | CZ47-03 | 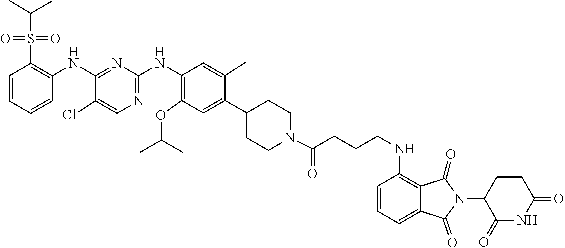 [SMILES: Cc1cc(Nc2ncc(Cl)c(Nc3ccccc3S(=O)(=O)C(C)C)n2)c(OC(C)C)cc1C1CCN(C(=O)CCCNc2cccc3c2C(=O)N(C2CCC(=O)NC2=O)C3=O)CC1] | 4-((4-(4-(4-((5- Chloro-4-((2- (isopropylsulfonyl) phenyl)amino) pyrimidin-2-yl) amino)-5- isopropoxy-2- methylphenyl) piperidin-1-yl)- 4-oxobutyl) amino)-(2-(2,6- dioxopiperidin-3- yl)-isoindoline- 1,3-dione |
| 25 | CZ47-04 | 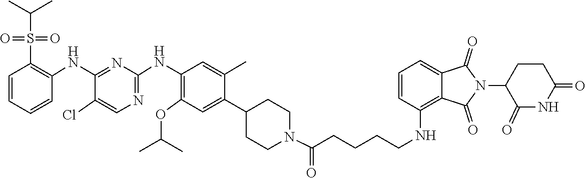 [SMILES: Cc1cc(Nc2ncc(Cl)c(Nc3ccccc3S(=O)(=O)C(C)C)n2)c(OC(C)C)cc1C1CCN(C(=O)CCCCNc2cccc3c2C(=O)N(C2CCC(=O)NC2=O)C3=O)CC1] | 4-((5-(4-(4-((5- Chloro-4-((2- (isopropylsulfonyl) phenyl)amino) pyrimidin-2-yl) amino)-5- isopropoxy-2- methylphenyl) piperidin-1-yl)- 5-oxopentyl) amino)-(2-(2,6- dioxopiperidin-3- |
| | | | yl)-isoindoline- |
| | | | 1,3-dione |
| 26 | CZ47-05 | 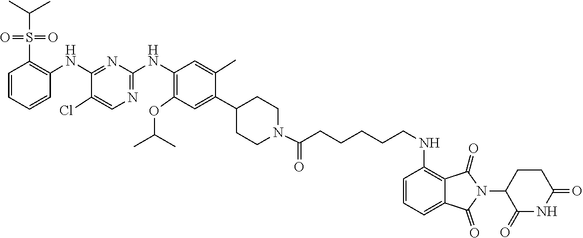 [SMILES: Cc1cc(Nc2ncc(Cl)c(Nc3ccccc3S(=O)(=O)C(C)C)n2)c(OC(C)C)cc1C1CCN(C(=O)CCCCCNc2cccc3c2C(=O)N(C2CCC(=O)NC2=O)C3=O)CC1] | 4-((6-(4-(4-((5- Chloro-4-((2- (isopropylsulfonyl) phenyl)amino) pyrimidin-2-yl) amino)-5- isopropoxy-2- methylphenyl) piperidin-1-yl)- 6-oxohexyl) amino)-(2-(2,6- dioxopiperidin-3- yl)-isoindoline- 1,3-dione |
| 27 | CZ47-06 | 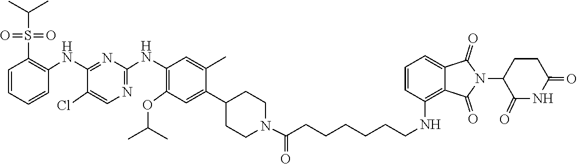 [SMILES: Cc1cc(Nc2ncc(Cl)c(Nc3ccccc3S(=O)(=O)C(C)C)n2)c(OC(C)C)cc1C1CCN(C(=O)CCCCCCNc2cccc3c2C(=O)N(C2CCC(=O)NC2=O)C3=O)CC1] | 4-((7-(4-(4-((5- Chloro-4-((2- (isopropylsulfonyl) phenyl)amino) pyrimidin-2-yl) amino)-5- isopropoxy-2- methylphenyl) piperidin-1-yl)- 7-oxoheptyl) amino)-(2-(2,6- |
| | | | dioxopiperidin-3- |
| | | | yl)-isoindoline- |
| | | | 1,3-dione |
| 28 | CZ47-07 | 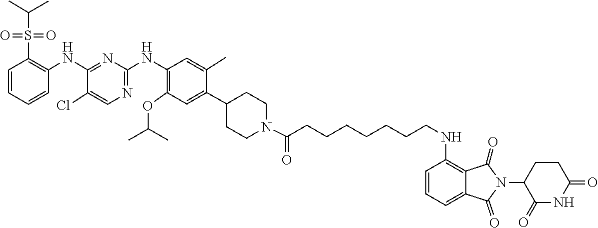 [SMILES: Cc1cc(Nc2ncc(Cl)c(Nc3ccccc3S(=O)(=O)C(C)C)n2)c(OC(C)C)cc1C1CCN(C(=O)CCCCCCCNc2cccc3c2C(=O)N(C2CCC(=O)NC2=O)C3=O)CC1] | 4-((8-(4-(4-((5- Chloro-4-((2- (isopropylsulfonyl) phenyl)amino) pyrimidin-2-yl) amino)-5- isopropoxy-2- methylphenyl) piperidin-1-yl)- 8-oxoocytyl) amino)-(2-(2,6- dioxopiperidin-3- yl)-isoindoline- 1,3-dione |
| 29 | CZ47-08 | 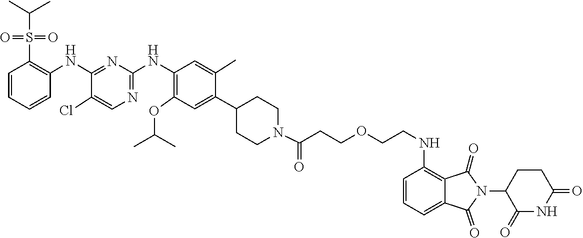 [SMILES: Cc1cc(Nc2ncc(Cl)c(Nc3ccccc3S(=O)(=O)C(C)C)n2)c(OC(C)C)cc1C1CCN(C(=O)CCOCCNc2cccc3c2C(=O)N(C2CCC(=O)NC2=O)C3=O)CC1] | 4-((2-(3-(4-(4-((5- Chloro-4-((2- (isopropylsulfonyl) phenyl)amino) pyrimidin-2-yl) amino)-5- isopropoxy-2- methylphenyl) piperidin-1-yl)- 3-oxopropoxy) ethyl)amino)- (2-(2,6- dioxopiperidin-3- yl)-isoindoline- 1,3-dione |
| 30 | CZ47-09 | 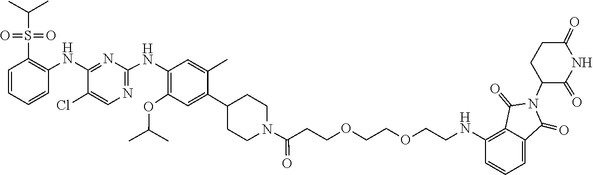 [SMILES: Cc1cc(Nc2ncc(Cl)c(Nc3ccccc3S(=O)(=O)C(C)C)n2)c(OC(C)C)cc1C1CCN(C(=O)CCOCCOCCNc2cccc3c2C(=O)N(C2CCC(=O)NC2=O)C3=O)CC1] | 4-((2-(2-(3-(4-(4- ((5-Chloro-4-((2- (isopropylsulfonyl) phenyl)amino) pyrimidin-2-yl) amino)-5- isopropoxy-2- methylphenyl) piperidin-1-yl)- 3-oxopropoxy) ethoxy)ethyl) |
| | | | amino)-(2-(2,6- |
| | | | dioxopiperidin-3- |
| | | | yl)-isoindoline- |
| | | | 1,3-dione |
| 31 | CZ47-10 | 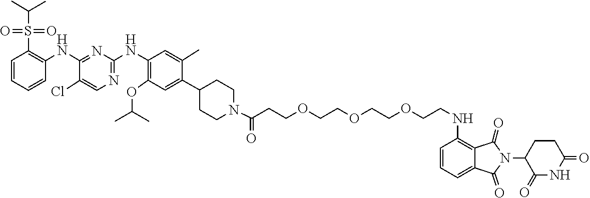 [SMILES: Cc1cc(Nc2ncc(Cl)c(Nc3ccccc3S(=O)(=O)C(C)C)n2)c(OC(C)C)cc1C1CCN(C(=O)CCOCCOCCOCCNc2cccc3c2C(=O)N(C2CCC(=O)NC2=O)C3=O)CC1] | 4-((2-(2-(2-(3-(4-(4- ((5-Chloro-4-((2- (isopropylsulfonyl) phenyl)amino) pyrimidin-2-yl) amino)-5- isopropoxy-2- methylphenyl) piperidin-1-yl)- 3-oxopropoxy) ethoxy)ethoxy) ethyl)amino)- (2-(2,6- |
| | | | dioxopiperidin-3- |
| | | | yl)-isoindoline- |
| | | | 1,3-dione |
| 32 | CZ47-11 | 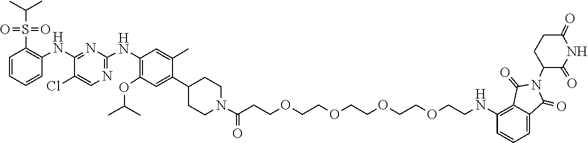 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCNc2cccc3c2C(=O)N(C2CCC(=O)NC2=O)C3=O)CC1] | 4-((15-(4-(4-((5- Chloro-4-((2- (isopropylsulfonyl) phenyl)amino) pyrimidin-2-yl) amino)-5- isopropoxy-2- methylphenyl) piperidin-1-yl)- |
| | | | 15-oxo-3,6,9,12- |
| | | | tetraoxapentadecyl) |
| | | | amino)-2-(2,6- |
| | | | dioxopiperidin-3- |
| | | | yl)-isoindoline- |
| | | | 1,3-dione |
| 33 | CZ47-12 | 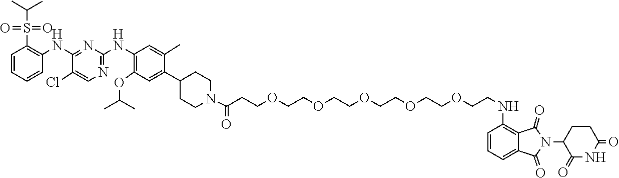 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCOCCNc2cccc3c2C(=O)N(C2CCC(=O)NC2=O)C3=O)CC1] | 4-((18-(4-(4-((5- Chloro-4-((2- (isopropylsulfonyl) phenyl)amino) pyrimidin-2-yl) amino)-5- isopropoxy-2- methylphenyl) piperidin-1-yl)- 18-oxo-3,6,9,12- pentaoxaotadecyl) |
| | | | amino)-2-(2,6- |
| | | | dioxopiperidin-3- |
| | | | yl)-isoindoline- |
| | | | 1,3-dione |
| 34 | CZ47-13 | 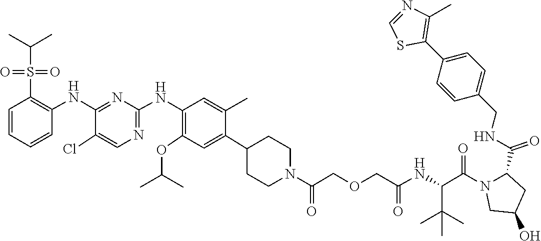 [SMILES: Cc1cc(Nc2ncc(Cl)c(Nc3ccccc3S(=O)(=O)C(C)C)n2)c(OC(C)C)cc1C1CCN(C(=O)COCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (2-(2-(4-(4-((5- Chloro-4-((2- (isopropylsulfonyl) phenyl)amino) pyrimidin-2-yl) amino)-5- isopropoxy-2- methylphenyl) piperidin-1-yl)-2 oxoethoxy) acetamido)-3,3- dimethylbutanoyl)- 4-hydroxy-N-(4-(4- methylthiazol-5- |
| | | | yl)benzyl) |
| | | | pyrrolidine-2- |
| | | | carboxamide |
| 35 | CZ47-14 | 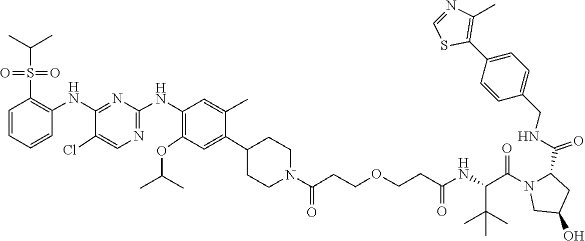 [SMILES: Cc1cc(Nc2ncc(Cl)c(Nc3ccccc3S(=O)(=O)C(C)C)n2)c(OC(C)C)cc1C1CCN(C(=O)CCOCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (3-(3-(4-(4-((5- Chloro-4-((2- (isopropylsulfonyl) phenyl)amino) pyrimidin-2-yl) amino)-5- isopropoxy-2- methylphenyl) piperidin-1-yl)-3 oxopropoxy) propanamido)-3,3- dimethylbutanoyl)- 4-hydroxy-N-(4-(4- methylthiazol-5- |
| | | | yl)benzyl) |
| | | | pyrrolidine-2- |
| | | | carboxamide |
| 36 | CZ47-15 | 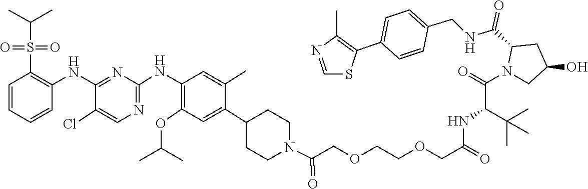 [SMILES: Cc1cc(Nc2ncc(Cl)c(Nc3ccccc3S(=O)(=O)C(C)C)n2)c(OC(C)C)cc1C1CCN(C(=O)COCCOCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (2-(2-(2-(4-(4-((5- Chloro-4-((2- (isopropylsulfonyl) phenyl)amino) pyrimidin-2-yl) amino)-5- isopropoxy-2- methylphenyl) piperidin-1-yl)-2 oxoethoxy)ethoxy) acetamido)-3,3- |
| | | | dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 37 | CZ47-16 | 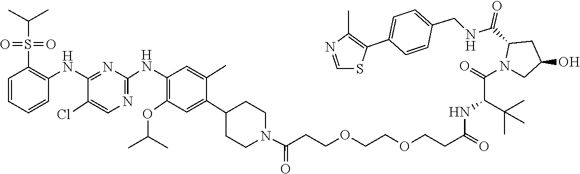 [SMILES: Cc1cc(Nc2ncc(Cl)c(Nc3ccccc3S(=O)(=O)C(C)C)n2)c(OC(C)C)cc1C1CCN(C(=O)CCOCCOCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (3-(2-(3-(4-(4-((5- Chloro-4-((2- (isopropylsulfonyl) phenyl)amino) pyrimidin-2-yl) amino)-5- isopropoxy-2- methylphenyl) piperidin-1-yl)-3 oxopropoxy)ethoxy) |
| | | | propanamido)-3,3- |
| | | | dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 38 | CZ47-17 | 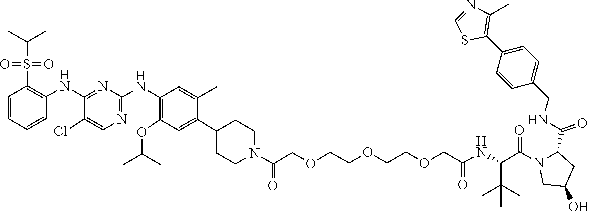 [SMILES: Cc1cc(Nc2ncc(Cl)c(Nc3ccccc3S(=O)(=O)C(C)C)n2)c(OC(C)C)cc1C1CCN(C(=O)COCCOCCOCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (tert-Butyl)-14-(4- (4-((5-chloro-4-((2- (isopropylsulfonyl) phenyl)amino) pyrimidin-2-yl) amino)-5- isopropoxy-2- methylphenyl) piperidin-1-yl)- 4,14-dioxo-6,9,12- trioxa-3- azatetradecanoyl)- |
| | | | 4-hydroxy-N-(4-(4- |
| | | | methylthiazol-5- |
| | | | yl)benzyl) |
| | | | pyrrolidine-2- |
| | | | carboxamide |
| 39 | CZ47-18 | 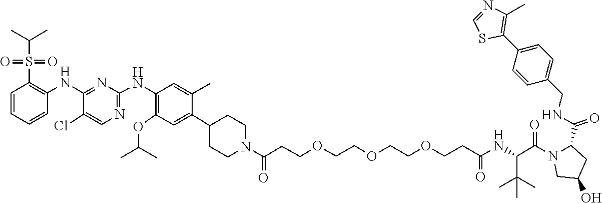 [SMILES: Cc1cc(Nc2ncc(Cl)c(Nc3ccccc3S(=O)(=O)C(C)C)n2)c(OC(C)C)cc1C1CCN(C(=O)CCOCCOCCOCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (tert-Butyl)-16-(4- (4-((5-chloro-4-((2- (isopropylsulfonyl) phenyl)amino) pyrimidin-2-yl) amino)-5- isopropoxy-2- methylphenyl) piperidin-1-yl)- 4,16-dioxo-7,10,13- trioxa-3- azahexadecanoyl)- |
| | | | 4-hydroxy-N-(4-(4- |
| | | | methylthiazol-5- |
| | | | yl)benzyl) |
| | | | pyrrolidine-2- |
| | | | carboxamide |
| 40 | CZ47-19 | 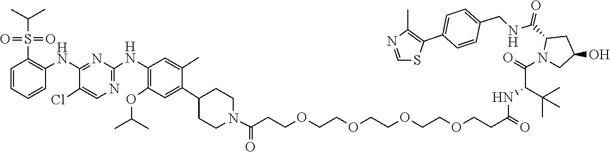 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (tert-Butyl)-19-(4- (4-((5-chloro-4-((2- (isopropylsulfonyl) phenyl)amino) pyrimidin-2-yl) amino)-5- isopropoxy-2- methylphenyl) piperidin-1-yl)- |
| | | | 4,19-dioxo-7,10,13- |
| | | | tetraoxa-3- |
| | | | azanonadecanoyl)- |
| | | | 4-hydroxy-N-(4-(4- |
| | | | methylthiazol-5- |
| | | | yl)benzyl) |
| | | | pyrrolidine-2- |
| | | | carboxamide |
| 41 | CZ47-20 | 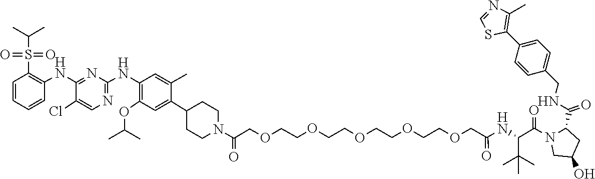 [SMILES: Cc1cc(Nc2ncc(Cl)c(Nc3ccccc3S(=O)(=O)C(C)C)n2)c(OC(C)C)cc1C1CCN(C(=O)COCCOCCOCCOCCOCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (tert-Butyl)-20-(4- (4-((5-chloro-4-((2- (isopropylsulfonyl) phenyl)amino) pyrimidin-2-yl) amino)-5- isopropoxy-2- methylphenyl) piperidin-1-yl)- 4,20-dioxo-6,9,12, |
| | | | 15,18-pentaoxa-3- |
| | | | azaicosanoyl)- |
| | | | 4-hydroxy-N-(4-(4- |
| | | | methylthiazol-5- |
| | | | yl)benzyl) |
| | | | pyrrolidine-2- |
| | | | carboxamide |
| 42 | CZ47-21 | 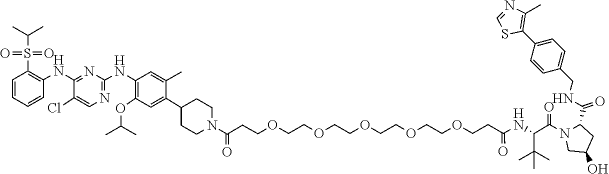 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCOCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (tert-Butyl)-22-(4- (4-((5-chloro-4-((2- (isopropylsulfonyl) phenyl)amino) pyrimidin-2-yl) amino)-5- isopropoxy-2- methylphenyl) piperidin-1-yl)- 4,22-dioxo-7,10,13, |
| | | | 16,19-pentaoxa-3- |
| | | | azadocosanoyl)- |
| | | | 4-hydroxy-N-(4-(4- |
| | | | methylthiazol-5- |
| | | | yl)benzyl) |
| | | | pyrrolidine-2- |
| | | | carboxamide |
| 43 | CZ47-22 | 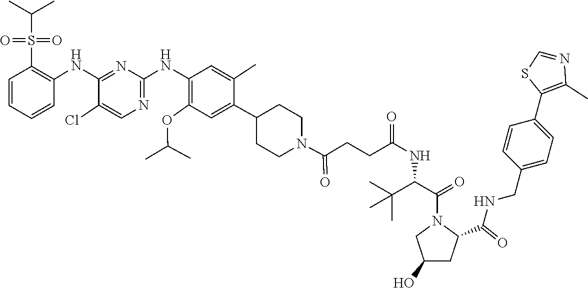 [SMILES: Cc1cc(Nc2ncc(Cl)c(Nc3ccccc3S(=O)(=O)C(C)C)n2)c(OC(C)C)cc1C1CCN(C(=O)CCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (4-(4-(4-((5- Chloro-4-((2- (isopropylsulfonyl) phenyl)amino) pyrimidin-2-yl) amino)-5- isopropoxy-2- methylphenyl) piperidin-1-yl)-4- oxobutanamido)- 3,3- dimethylbutanoyl)- 4-hydroxy-N-(4-(4- methylthiazol-5- yl)benzyl) pyrrolidine-2- carboxamide |
| 44 | CZ47-23 | 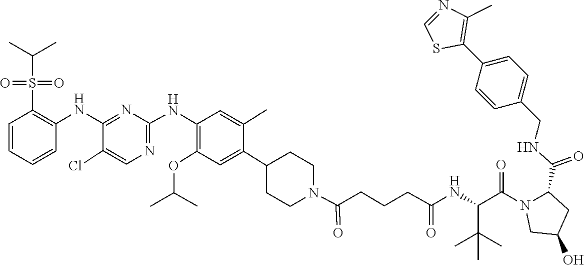 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (5-(4-(4-((5- Chloro-4-((2- (isopropylsulfonyl) phenyl)amino) pyrimidin-2-yl) amino)-5- isopropoxy-2- methylphenyl) piperidin-1-yl)-5- oxopentanamido)-3,3- dimethylbutanoyl)- 4-hydroxy-N-(4-(4- methylthiazol-5- yl)benzyl) pyrrolidine-2- |
| | | | carboxamide |
| 45 | CZ47-24 | 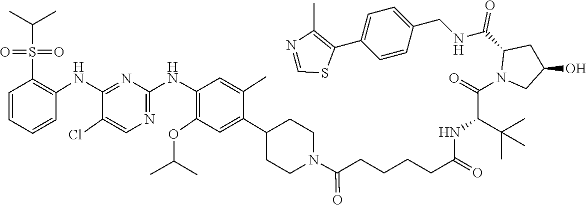 [SMILES: Cc1cc(Nc2ncc(Cl)c(Nc3ccccc3S(=O)(=O)C(C)C)n2)c(OC(C)C)cc1C1CCN(C(=O)CCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (6-(4-(4-((5- Chloro-4-((2- (isopropylsulfonyl) phenyl)amino) pyrimidin-2-yl) amino)-5- isopropoxy-2- methylphenyl) piperidin-1-yl)-6- oxohexanamido)-3,3- dimethylbutanoyl)- 4-hydroxy-N-(4-(4- |
| | | | methylthiazol-5- |
| | | | yl)benzyl) |
| | | | pyrrolidine-2- |
| | | | carboxamide |
| 46 | CZ47-25 | 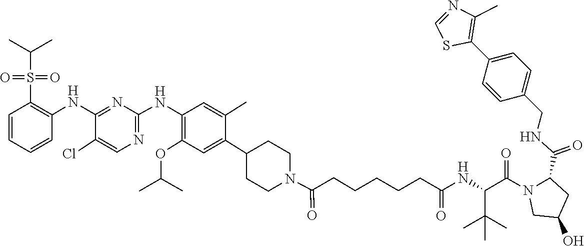 [SMILES: Cc1cc(Nc2ncc(Cl)c(Nc3ccccc3S(=O)(=O)C(C)C)n2)c(OC(C)C)cc1C1CCN(C(=O)CCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (7-(4-(4-((5- Chloro-4-((2- (isopropylsulfonyl) phenyl)amino) pyrimidin-2-yl) amino)-5- isopropoxy-2- methylphenyl) piperidin-1-yl)-6- oxoheptanamido)-3,3- dimethylbutanoyl)- 4-hydroxy-N-(4-(4- methylthiazol-5- yl)benzyl) |
| | | | pyrrolidine-2- |
| | | | carboxamide |
| 47 | CZ47-26 | 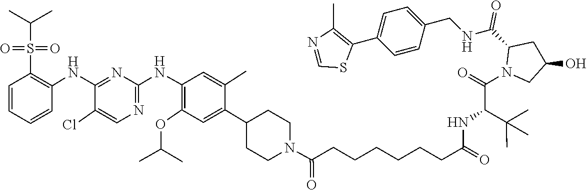 [SMILES: Cc1cc(Nc2ncc(Cl)c(Nc3ccccc3S(=O)(=O)C(C)C)n2)c(OC(C)C)cc1C1CCN(C(=O)CCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (8-(4-(4-((5- Chloro-4-((2- (isopropylsulfonyl) phenyl)amino) pyrimidin-2-yl) amino)-5- isopropoxy-2- methylphenyl) piperidin-1-yl)-8- oxooctanamido)-3,3- dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 48 | CZ47-27 | 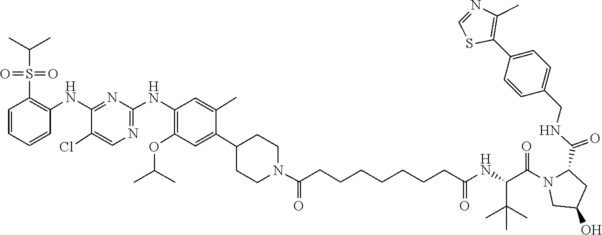 [SMILES: Cc1cc(Nc2ncc(Cl)c(Nc3ccccc3S(=O)(=O)C(C)C)n2)c(OC(C)C)cc1C1CCN(C(=O)CCCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (9-(4-(4-((5- Chloro-4-((2- (isopropylsulfonyl) phenyl)amino) pyrimidin-2-yl) amino)-5- isopropoxy-2- methylphenyl) piperidin-1-yl)-9- oxononanamido)-3,3- dimethylbutanoyl)- 4-hydroxy-N-(4-(4- methylthiazol-5- yl)benzyl) |
| | | | pyrrolidine-2- |
| | | | carboxamide |
| 49 | CZ47-28 | 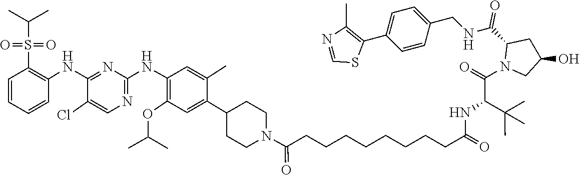 [SMILES: Cc1cc(Nc2ncc(Cl)c(Nc3ccccc3S(=O)(=O)C(C)C)n2)c(OC(C)C)cc1C1CCN(C(=O)CCCCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (10-(4-(4-((5- Chloro-4-((2- (isopropylsulfonyl) phenyl)amino) pyrimidin-2-yl) amino)-5- isopropoxy-2- methylphenyl) piperidin-1-yl)-10- oxodecanamido)- |
| | | | 3,3- |
| | | | dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 50 | CZ47-29 | 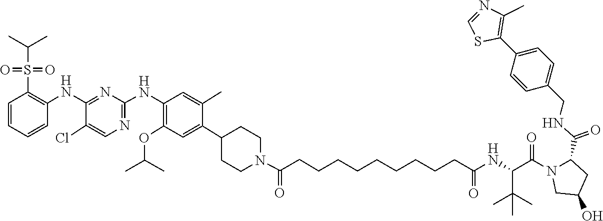 [SMILES: Cc1cc(Nc2ncc(Cl)c(Nc3ccccc3S(=O)(=O)C(C)C)n2)c(OC(C)C)cc1C1CCN(C(=O)CCCCCCCCCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (11-(4-(4-((5- Chloro-4-((2- (isopropylsulfonyl) phenyl)amino) pyrimidin-2-yl) amino)-5- isopropoxy-2- methylphenyl) piperidin-1-yl)-11- oxoundecanamido)- 3,3- dimethylbutanoyl)- 4-hydroxy-N-(4-(4- |
| | | | methylthiazol-5- |
| | | | yl)benzyl) |
| | | | pyrrolidine-2- |
| | | | carboxamide |
| 51 | CZ47-40 | 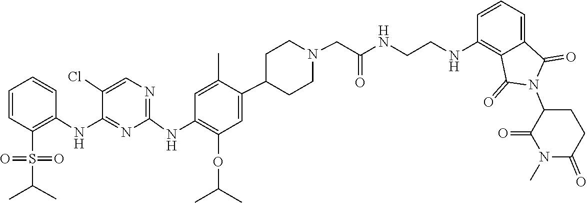 [SMILES: Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C)C2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) piperidin-1-yl)-N- (2-((2-(1-methyl- 2,6-dioxopiperidin- 3-yl)-1,3- dioxoisoindolin-4- |
| | | | yl)amino)ethyl) |
| | | | acetamide |
| 52 | CZ47-48 | 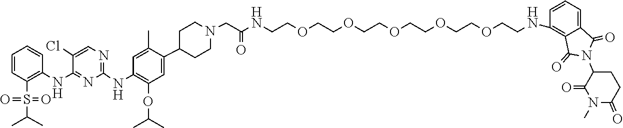 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCOCCNc2cccc3c2C(=O)N(C2CCC(=O)N(C)C2=O)C3=O)CC1] | 2-(4-(4-((5-Chloro- 4-((2- (isopropylsulfonyl) phenyl)amino) pyrimidin-2-yl) amino)-5- isopropoxy-2- methylphenyl) |
| | | | piperidin-1-yl)-N- |
| | | | (17-((2-(1-methyl- |
| | | | 2,6-dioxopiperidin- |
| | | | 3-yl)-1,3- |
| | | | dioxoisoindolin-4- |
| | | | yl)amino)- |
| | | | 3,6,9,12,15- |
| | | | pentaoxaheptadecy) |
| | | | acetamide |
| 53 | HC58-98 | 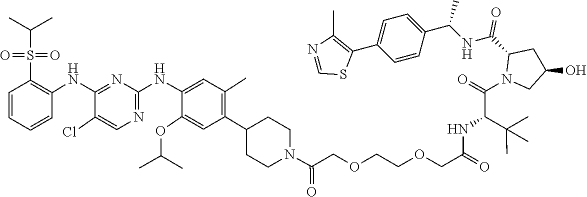 [SMILES: Cc1cc(Nc2ncc(Cl)c(Nc3ccccc3S(=O)(=O)C(C)C)n2)c(OC(C)C)cc1C1CCN(C(=O)COCCOCC(=O)N[C@H](C(=O)N2C[C@H](O)C[C@H]2C(=O)N[C@@H](C)c2ccc(-c3scnc3C)cc2)C(C)(C)C)CC1] | (2S,4R)-1-((S)-2- (2-(2-(2-(4-(4-((5- chloro-4-((2- (isopropylsulfonyl) phenyl)amino) pyrimidin-2-yl) amino)-5- isopropoxy-2- methylphenyl) piperidin-1-yl)-2- oxoethoxy)ethoxy) acetamido)-3,3- dimethylbutanoyl)- |
| | | | 4-hydroxy-N-((S)- |
| | | | 1-(4-(4- |
| | | | methylthiazol-5- |
| | | | yl)phenyl)ethyl) |
| | | | pyrrolidine-2- |
| | | | carboxamide |
| 54 | HC58-99 | 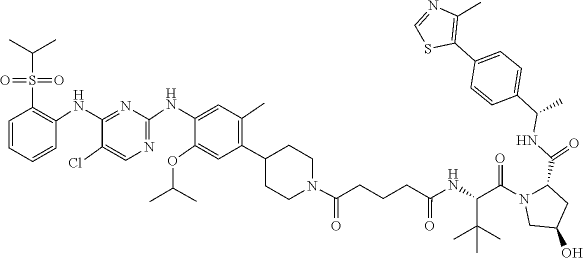 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@H](O)C[C@H]2C(=O)N[C@@H](C)c2ccc(-c3scnc3C)cc2)C(C)(C)C)CC1] | (2S,4R)-1-((S)-2- (5-(4-(4-((5- chloro-4-((2- (isopropylsulfonyl) phenyl)amino) pyrimidin-2-yl) amino)-5- isopropoxy-2- methylphenyl) piperidin-1-yl)-5- oxopentanamido)- 3,3- dimethylbutanoyl)- 4-hydroxy-N-((S)-1-(4-(4-methylthiazol-5- yl)phenyl)ethyl) |
| | | | pyrrolidine-2- |
| | | | carboxamide |
| 55 | HC58-100 | 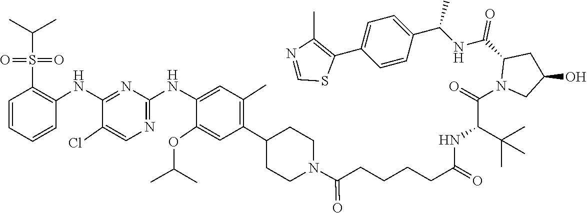 [SMILES: Cc1cc(Nc2ncc(Cl)c(Nc3ccccc3S(=O)(=O)C(C)C)n2)c(OC(C)C)cc1C1CCN(C(=O)CCCCC(=O)N[C@H](C(=O)N2C[C@H](O)C[C@H]2C(=O)N[C@@H](C)c2ccc(-c3scnc3C)cc2)C(C)(C)C)CC1] | (2S,4R)-1-((S)-2- (6-(4-(4-((5- chloro-4-((2- (isopropylsulfonyl) phenyl)amino) pyrimidin-2-yl) amino)-5- isopropoxy-2- methylphenyl) piperidin-1-yl)-6- oxohexanamido)- 3,3- dimethylbutanoyl)- 4-hydroxy-N-((S)- |
| | | | 1-(4-(4- |
| | | | methylthiazol-5- |
| | | | yl)phenyl)ethyl) |
| | | | pyrrolidine-2- |
| | | | carboxamide |
| 56 | HC58-110 | 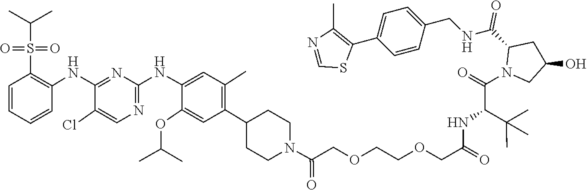 [SMILES: Cc1cc(Nc2ncc(Cl)c(Nc3ccccc3S(=O)(=O)C(C)C)n2)c(OC(C)C)cc1C1CCN(C(=O)COCCOCC(=O)N[C@H](C(=O)N2C[C@H](O)C[C@H]2C(=O)NCc2ccc(-c3scnc3C)cc2)C(C)(C)C)CC1] | (2S,4R)-1-((S)-2- (2-(2-(2-(4-(4-((5- chloro-4-((2- (isopropylsulfonyl) phenyl)amino) pyrimidin-2-yl) amino)-5- isopropoxy-2- methylphenyl) piperidin-1-yl)-2- oxoethoxy)ethoxy) acetamido)-3,3- |
| | | | dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |
| 57 | HC58-111 | 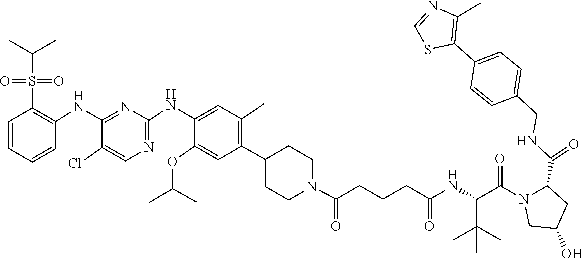 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@@H](O)C[C@H]2C(=O)NCc2ccc(-c3scnc3C)cc2)C(C)(C)C)CC1] | (2S,4R)-1-((S)-2- (5-(4-(4-((5- chloro-4-((2- (isopropylsulfonyl) phenyl)amino) pyrimidin-2-yl) amino)-5- isopropoxy-2- methylphenyl) piperidin-1-yl)-5- oxopentanamido)- 3,3- dimethylbutanoyl)- 4-hydroxy-N-(4-(4-methylthiazol-5- yl)benzyl) |
| | | | pyrrolidine-2- |
| | | | carboxamide |
| 58 | HC58-112 | 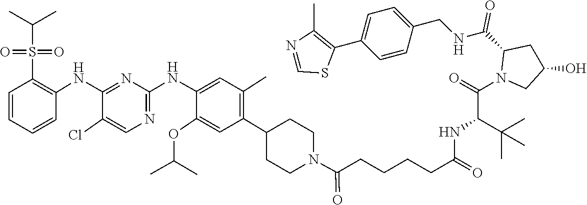 [SMILES: Cc1cc(Nc2ncc(Cl)c(Nc3ccccc3S(=O)(=O)C(C)C)n2)c(OC(C)C)cc1C1CCN(C(=O)CCCCC(=O)N[C@H](C(=O)N2C[C@@H](O)C[C@H]2C(=O)NCc2ccc(-c3scnc3C)cc2)C(C)(C)C)CC1] | (2S,4R)-1-((S)-2- (6-(4-(4-((5- chloro-4-((2- (isopropylsulfonyl) phenyl)amino) pyrimidin-2-yl) amino)-5- isopropoxy-2- methylphenyl) piperidin-1-yl)-6- oxohexanamido)- 3,3- dimethylbutanoyl)- |
| | | | 4-hydroxy-N-(4-(4- |
| | | | methylthiazol-5- |
| | | | yl)benzyl) |
| | | | pyrrolidine-2- |
| | | | carboxamide |

In some aspects, the ALK degraders/disruptors have the form “PI-Linker-EL”, as shown below:

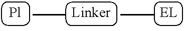

wherein PI comprises an ALK ligand (e.g., an ALK inhibitor) and EL comprises a degradation/disruption tag (e.g., E3 ligase ligand). Exemplary ALK ligands (PI), exemplary degradation/disruption tags (EL), and exemplary linkers (Linker) are illustrated below:
PI includes but is not limited to:

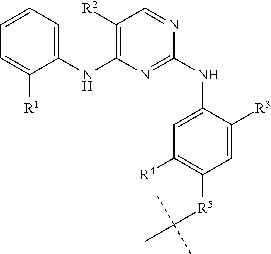

wherein
R¹ is (CR⁶R⁷)ₙSO₂R⁸, (CR⁶R⁷)ₙSO₂NR⁸R⁹, (CR⁶R⁷)ₙCOR⁸, (CR⁶R⁷)ₙCO₂R⁸, (CR⁶R⁷)ₙCONR⁸R⁹. (CR⁶R⁷)ₙP(O)R⁸R⁹, (CR⁶R⁷)ₙP(O)NR⁸R⁹;
R², R³ and R⁴ are independently hydrogen, halogen, C1-C8 alkyl, C1-C8 alkoxy, C1-C8 alkoxy alkyl;
R⁵ are independently hydrogen, halogen, C1-C8 alkyl, C1-C8 alkoxy, C1-C8 alkoxy alkyl, C1-C8 haloalkyl, C1-C8 hydroxyalkyl, C3-C7 cycloalkyl, C3-C7 heterocyclyl, C2-C8 alkenyl, C2-C8 alkynyl, OR¹⁰, SR¹⁰, NR¹⁰R¹¹, CN, NO₂, (CR¹⁰R¹¹)mNR¹²R¹³, (CR¹⁰R¹¹)mC(O)R¹², (NR¹⁰R¹¹)mNR¹²R¹³, (NR¹⁰R¹¹)mC(O)R¹², COR¹⁰, CO₂R¹⁰, CONR¹⁰R¹¹, NR¹⁰COR¹¹, NR¹⁰SOR¹¹, NR¹⁰SO₂R¹¹, SOR¹⁰, SO₂R¹⁰, SO₂NR¹⁰R¹¹, (CR¹⁰R¹¹)m-aryl, or (CR¹⁰R¹¹)m-heteroaryl; wherein
m=0-8;
n=0-3;
R⁶, R⁷, R⁸, R⁹, R¹⁰, R¹¹, R¹², R¹³ are independently hydrogen, C1-C8 alkyl, C1-C8 alkoxy, C2-C8 alkenyl, C2-C8 alkynyl, arylalkyl, cycloalkyl, heterocycloalkyl, aryl, heteroaryl, or heteroarylalkyl; and
R⁶ and R⁷, R⁸ and R⁹, R¹⁰ and R¹¹, R¹² and R¹³ can independently form 4-8 membered alkyl or heterocyclyl rings.

The ALK ligand can be a ALK inhibitor, such as, for example, crizotinib, ceritinib, alectinib, brigatinib, lorlatinib, TPX-0005, belizatinib, ensartinib, CEP-37440, and/or analogs thereof.

In some embodiments, the ALK ligand can be, e.g.,

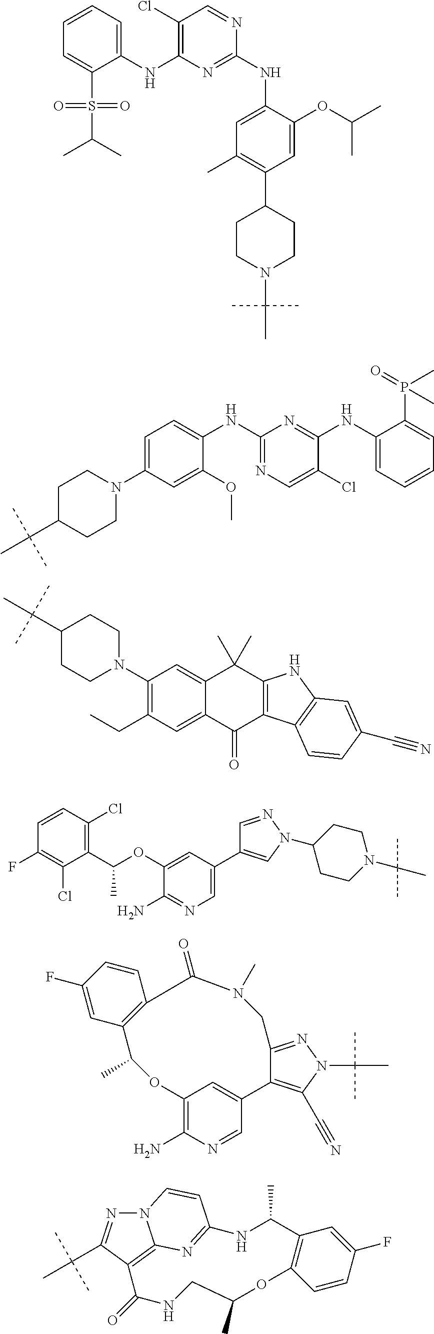

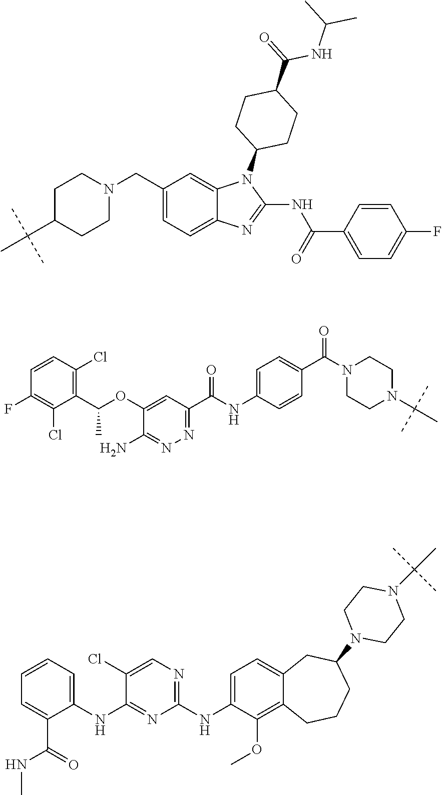

The ALK ligand can be bound to ALK, ALK fusion proteins, and/or ALK mutant proteins.

EL includes but is not limited to:

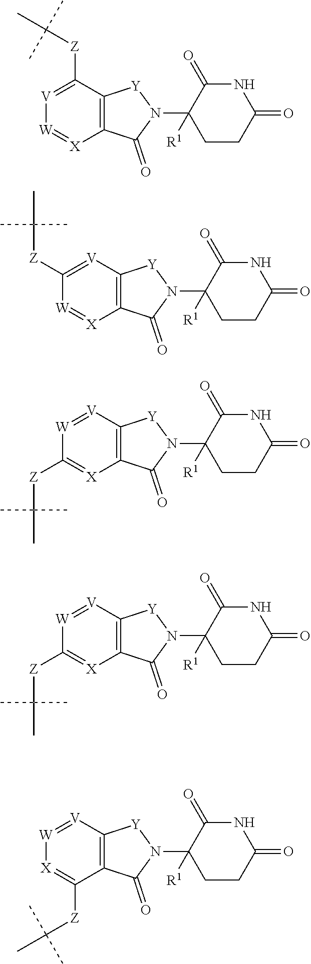

wherein
V, W, X are independently CR², or N;

### Y is CO or CH₂;

### Z is CH₂, NH, or O;

R¹ is hydrogen, methyl, or fluoro; and
R² is hydrogen, halogen, or C1-C5 alkyl;

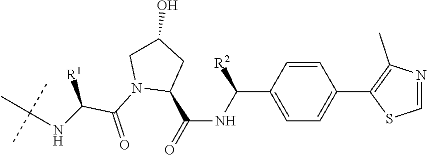

wherein
R¹ and R² are independently hydrogen, C1-C8 alkyl, C1-C8 alkoxyalkyl, C1-C8 haloalkyl, C1-C8 hydroxyalkyl, C3-C7 cycloalkyl, C3-C7 heterocyclyl, C2-C8 alkenyl, or C2-C8 alkynyl; and

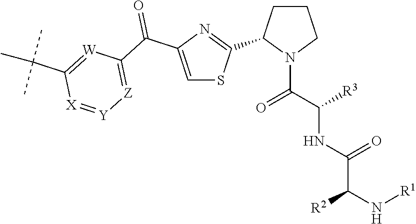

wherein
R¹, R², R³ and R⁴ are independently hydrogen, C1-C8 alkyl, C1-C8 alkoxyalkyl, C1-C8 haloalkyl, C1-C8 hydroxyalkyl, C3-C7 cycloalkyl, C3-C7 heterocyclyl, C2-C8 alkenyl, or C2-C8 alkynyl; and
V, W, X, Z are independently CR⁴, or N.

In some embodiments, the degradation/disruption tag can be for example, pomalidomide, thalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, and/or analogs thereof.

In some embodiments, the degradation/disruption tag can be, e.g.,

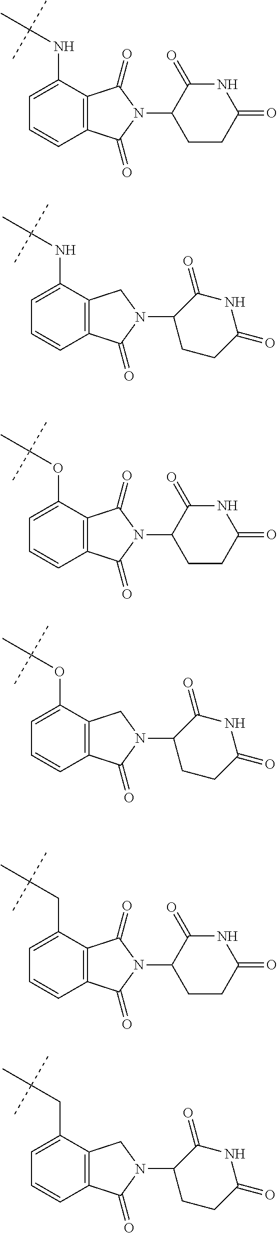

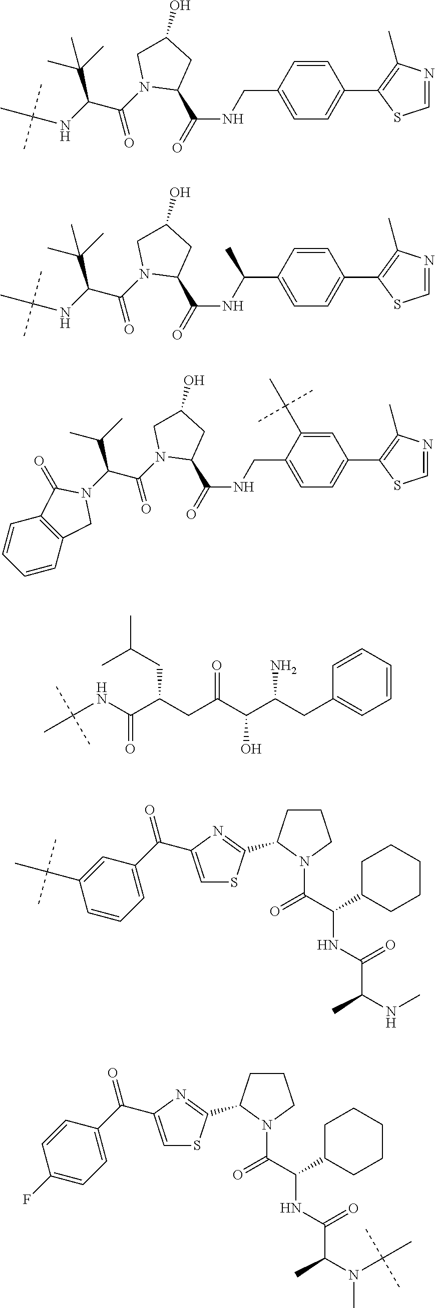

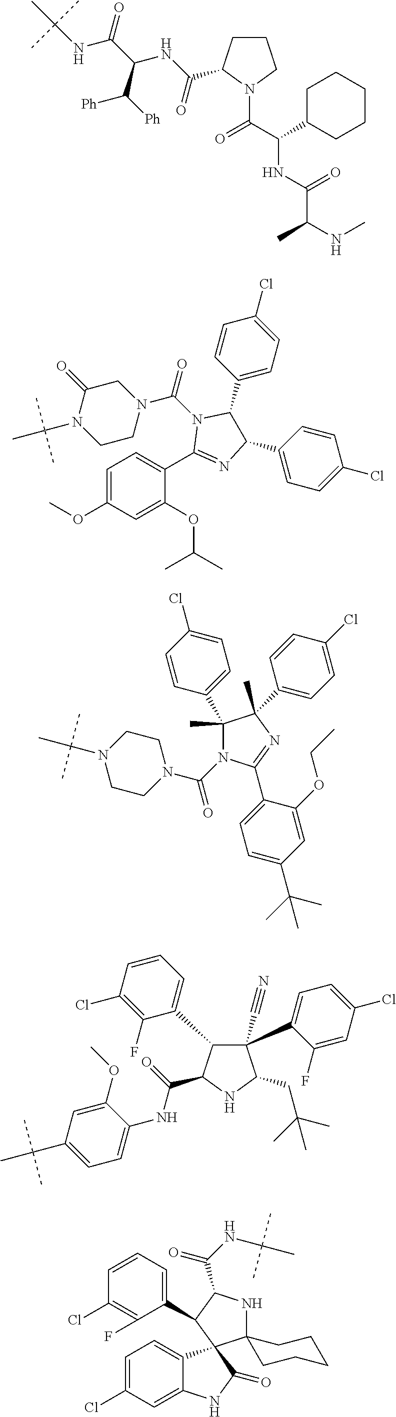

In some embodiments, the degradation/disruption tag can bind to a ubiquitin ligase (e.g., an E3 ligase such as a cereblon E3 ligase, a VHL E3 ligase, a MDM2 ligase, a TRIM21 ligase, a TRIM24 ligase, and/or a IAP ligase) and/or serve as a hydrophobic group that leads to ALK protein misfolding.

In any of the above-described compounds, the ALK ligand can be conjugated to the degradation/disruption tag through a linker. The linker can include, for example, acyclic or cyclic saturated or unsaturated carbon, ethylene glycol, amide, amino, ether, urea, carbamate, aromatic, heteroaromatic, heterocyclic and/or carbonyl containing groups with different lengths.

In some embodiments, the linker can be a moiety of:

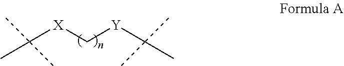

wherein

### X is C═O or CH₂;

### Y is C═O or CH₂; and

n is 0-15;

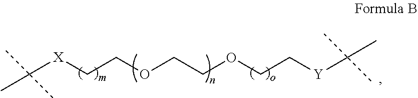

wherein

### X is C═O or CH₂;

### Y is C═O or CH₂;

m is 0-15;
n is 0-6; and
o is 0-15.

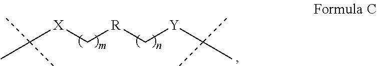

wherein

### X is C═O or CH₂;

### Y is C═O or CH₂;

R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl)-, —C═C—, —O—, —NH—, —N(C₁₋₃ alkyl)-, —C(O)NH—, —C(O)N(C₁₋₃ alkyl)-, a 3-13 membered ring, a 3-13 membered fused ring, a 3-13 membered bridged ring, and/or a 3-13 membered spiro ring;
m is 0-15; and
n is 0-15.

In some embodiments of Formula C, R is a 3-13 membered ring, a 3-13 membered fused ring, a 3-13 membered bridged ring, and/or a 3-13 membered spiro ring, one or more of which can contain one or more heteroatoms.

In some embodiments of Formula C, R has a structure of

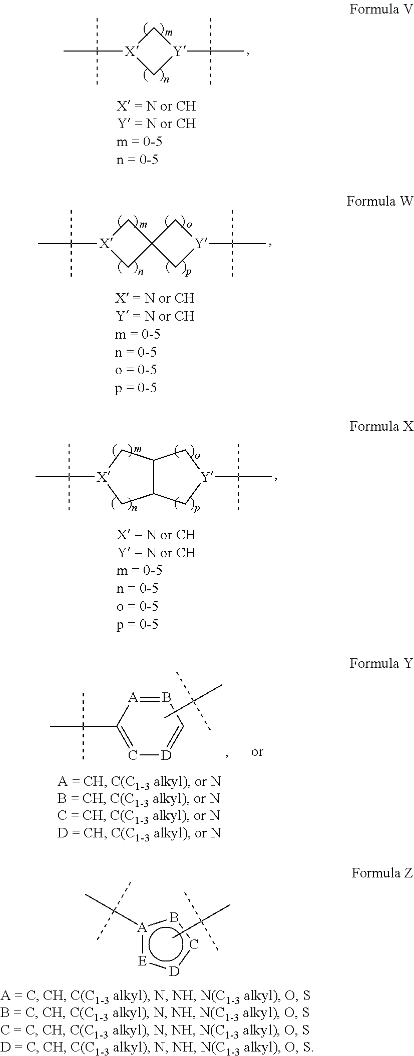

### Synthesis and Testing of Bivalent Compounds

The binding affinity of novel synthesized bivalent compounds (i.e., ALK degraders/disruptors) can be assessed using standard biophysical assay s known in the art (e.g., ITC). Cellular assays can then be used to assess the bivalent compound's ability to induce ALK degradation and inhibit cancer cell proliferation. Besides evaluating bivalent compound's-induced changes in the protein expression of ALK, ALK fusion proteins, or ALK mutant proteins, enzymatic activity can also be assessed. Assays suitable for use in any or all of these steps are known in the art, and include, e.g., Western blotting, quantitative mass spectrometry (MS) analysis, flow cytometry, enzymatic inhibition, ITC, SPR, cell growth inhibition and xenograft and PDX models. Suitable cell lines for use in any or all of these steps are known in the art and include, e.g., ALK-positive diffused large cell lymphoma cell lines [e.g., SU-DHL-1 that contain the t(2;5) (p23;q35) chromosomal translocation and express 80 kDaNPM-ALK fusion protein, p80 (Pulford et al., 1997) (Wood et al., 1996)] and ALK-positive non-small cell lung cancer cell lines [e.g., NCI-H2228 that contains an EML4-ALK v3 fusion (Choi et al., 2008; Koivunen et al., 2008)].

By way of non-limiting example, detailed synthesis protocols are described in the Examples for specific exemplary ALK degraders/disruptors.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or cfc-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

### Characterization of Exemplary ALK Degraders/Disruptors

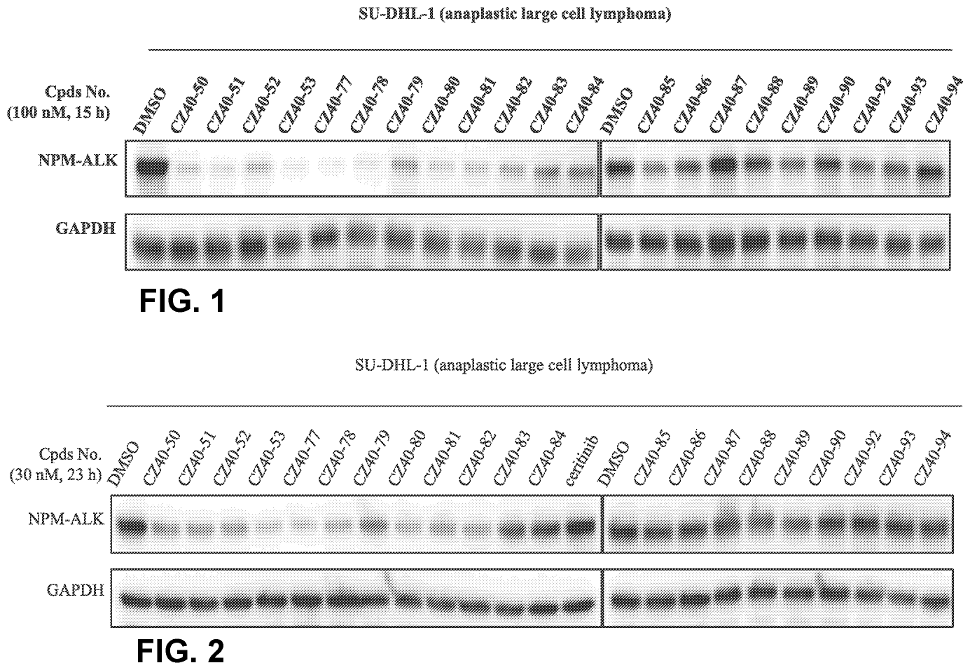

Specific exemplary ALK degraders/disruptors were characterized in various different anaplastic large cell lymphoma (ALCL) cells and non-small cell lung cancer (NSCLC) cells (Examples 4-19, FIGS. 1-18). CZ40-53, CZ40-77, CZ40-78, CZ47-15, CZ47-23, CZ47-24, HC58-98, HC58-99, and HC58-100 in particular were found to be especially effective in suppressing both ALK expression and ALK activity. This efficacy in suppressing ALK expression and ALK activity correlated with efficacy in inhibiting cancer cell proliferation. In addition, CZ40-78 is bioavailable in mice.

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents known in the art (e.g., HSP90 inhibitors, IGF-1R inhibitors, HDM-2/p53 inhibitor, CDK inhibitor, and mTOR inhibitors). When co-administered, ALK degraders/disruptors disclosed herein can operate in conjunction with conventional chemotherapeutic agents to produce mechanistically additive or synergistic therapeutic effects.

In some aspects, the pH of the compositions disclosed herein can be adjusted with pharmaceutically acceptable acids, bases, or buffers to enhance the stability of the ALK degraders/disruptor or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, poly acrylates, waxes, poly ethylene-poly oxypropylene-block polymers, polyethylene glycol and wool fat. Cyclodextrins such as α-, β-, and γ-cyclodextrin, may also be advantageously used to enhance delivery of compounds of the formulae described herein.

As used herein, the ALK degraders/disruptors disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The ALK degraders/disruptors disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the ALK degraders/disruptors disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., alkali metal (e.g., sodium), alkaline earth metal (e.g., magnesium), ammonium and N-(alkyl)4+ salts. The invention also envisions the quaternization of any basic nitrogen-containing groups of the ALK degraders/disruptors disclosed herein. Water or oil-soluble or dispersible products can be obtained by such quaternization.

In some aspects, the pharmaceutical compositions disclosed herein can include an effective amount of one or more ALK degraders/disruptors. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in anon-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein (e.g., an ALK degraders/disruptors) and one or more additional compounds (e.g., one or more additional compounds, drugs, or agents used for the treatment of cancer or any other condition or disease, including conditions or diseases known to be associated with or caused by cancer), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the deseed or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising an ALK degrader/disruptor, including pharmaceutical compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering (e.g., an ALK-mediated cancer). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

As used herein, the term “treating cancer” means causing a partial or complete decrease in the rate of growth of a tumor, and/or in the size of the tumor and/or in the rate of local or distant tumor metastasis, and/or the overall tumor burden in a subject, and/or any decrease in tumor survival, in the presence of a degrader/disruptor (e.g., an ALK degrader/disruptor) described herein.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. Exemplary ALK-mediated cancers that can be treated with ALK degraders/disruptors include, for example, anaplastic large cell lymphoma; non-Hodgkin's lymphoma; an inflammatory myofibrolastic tumor; a neuroblastoma; sarcoma; lung, non-small cell lung cancer; bronchus; prostate; breast (including sporadic breast cancers and sufferers of Cowden disease); pancreas; gastrointestinal cancer; colon; rectum; colon carcinoma; colorectal adenoma; esophageal cancer, thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary: multiple myeloma; esophagus; a leukemia; acute myelogenous leukemia; chronic myelogenous leukemia; lymphocytic leukemia; lymphoma; myeloid leukemia; brain; a carcinoma of the brain; oral cavity and pharynx; larynx; rhabdomyosarcoma; spitz cancer, small intestine; and melanoma.

As used herein, the term “preventing a disease” (e.g., preventing cancer) in a subject means for example, to stop the development of one or more symptoms of a disease in a subject before they occur or are detectable, e.g., by the patient or the patient's doctor. Preferably, the disease (e.g., cancer) does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

### EXAMPLES

### Example 1. Synthesis of 2-(4-(4-((5-chloro-4-((2-(isopropylsulfonyl)phenyl)amino)pyrimidin-2-yl)amino)-5-isopropoxy-2-methylphenyl)piperidin-1-yl)acetic acid

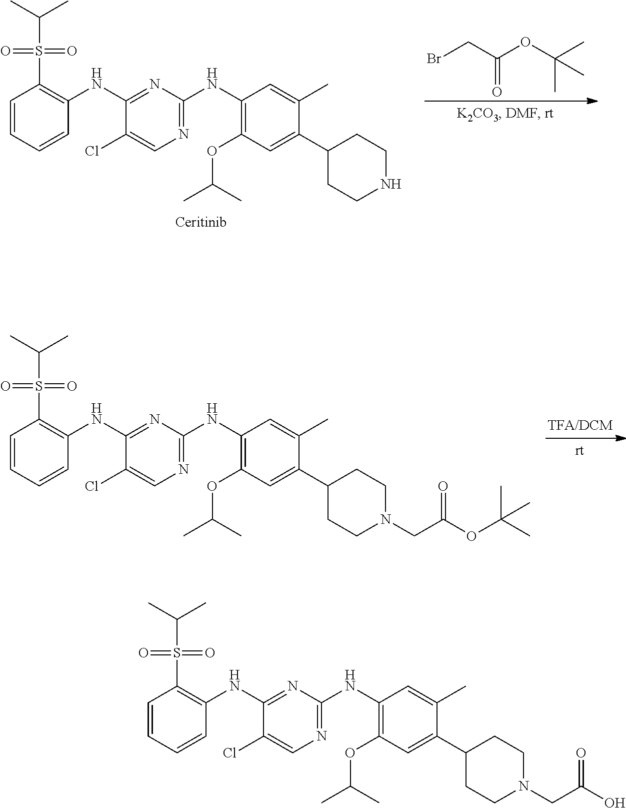

To a solution of ceritinib (500 mg, 0.896 mmol) and potassium carbonate (247 mg, 1.79 mmol) in DMF (5 mL) was added tert-butyl 2-bromoacetate (0.17 mL, 1.16 mmol). After being stirred at room temperature overnight, the reaction was quenched with water (15 mL) and extracted with dichloromethane (3×20 mL). The combined organic phase was dried over anhydrous sodium sulfate, filtered and purified by column chromatography on silica gel (dichloromethane methanol=20:1) to give the product as sticky oil (452 mg, yield 75%). ESI m/z=672.3 [M+H⁺]

tert-Butyl 2-(4-(4-((5-chloro-4-((2-(isopropylsulfonyl)phenyl)amino)pyrimidin-2-yl)amino)-5-isopropoxy-2-methylphenyl)piperidin-1-yl)acetate (450 mg, 0.67 mmol) was dissolved in dichloromethane (5 mL) and trifluroacetic acid (5 mL). After being stirred at room temperature for 5 h, the solvents were removed under reduced pressure. The residue was used in the next step without further purification. ESI m/z=616.2 [M+H⁺].

### Example 2. Synthesis of Representative Linker-EL Moieties

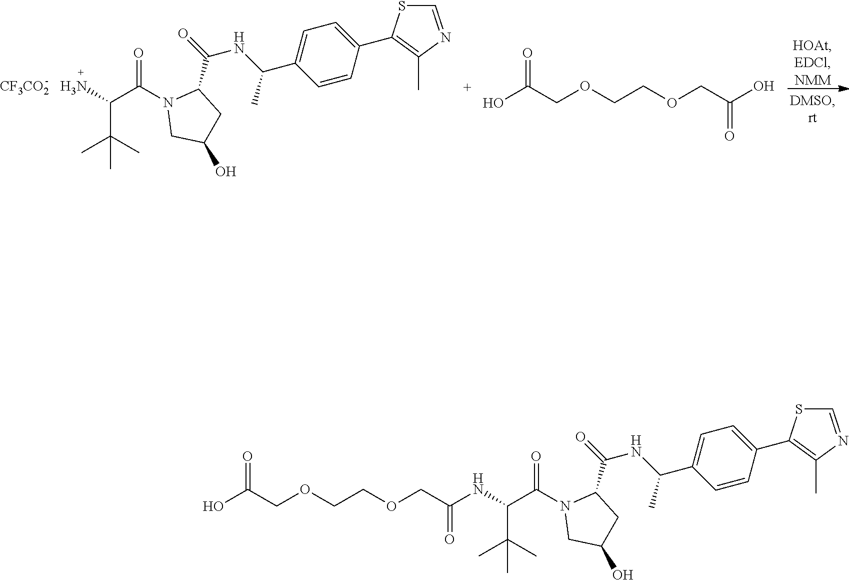 [SMILES: "CF3CO2" ; Cc1ncsc1-c1ccc([C@H](C)NC(=O)[C@@H]2C[C@@H](O)CN2C(=O)[C@@H](N)C(C)(C)C)cc1 ; O=C(O)COCCOCC(=O)O ; "HOAt,EDCl,NMM" ; Cc1ncsc1-c1ccc([C@H](C)NC(=O)[C@@H]2C[C@@H](O)CN2C(=O)[C@@H](NC(=O)COCCOCC(=O)O)C(C)(C)C)cc1]

Synthesis of 2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetic acid. To a solution of (S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-aminium 2,2,2-trifluoroacetate (20.0 mg, 0.037 mmol) and 2,2′-(ethane-1,2-diylbis(oxy))diacetic acid (7.9 mg, 0.044 mmol) in DMSO (1.0 mL) were added NMM (7.5 mg, 0.074 mmol), EDCI (10.6 mg, 0.056 mmol) and HOAt (7.6 mg, 0.056 mmol). The reaction mixture was stirred at room temperature overnight and purified by prepared HPLC to give the desired product HC58-94 as white solid (10.4 mg, yield 46%). ESI m/z=605.3 [M+H⁺], Following the above procedures for HC58-94, the following Linker-EL moieties were synthesized.

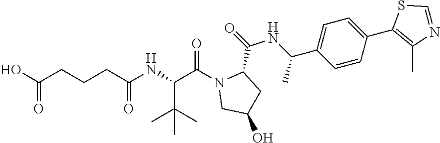 [SMILES: Cc1ncsc1-c1ccc([C@H](C)NC(=O)[C@@H]2C[C@@H](O)CN2C(=O)[C@@H](NC(=O)CCCC(=O)O)C(C)(C)C)cc1]

5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid (HC58-95). White solid, yield 56%. ESI m/z=559.3[M+H⁺].

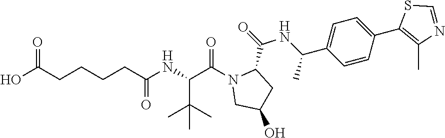 [SMILES: Cc1ncsc1-c1ccc([C@H](C)NC(=O)[C@@H]2C[C@@H](O)CN2C(=O)[C@@H](NC(=O)CCCCC(=O)O)C(C)(C)C)cc1]

6-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoic acid (HC58-96). White solid, yield 57%. ESI m/z=573.3[M+H⁺].

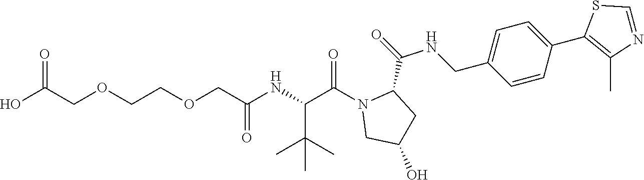 [SMILES: Cc1ncsc1-c1ccc(CNC(=O)[C@@H]2C[C@H](O)CN2C(=O)[C@@H](NC(=O)COCCOCC(=O)O)C(C)(C)C)cc1]

2-(2-(2-(((S)-1-((2S,4S)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetic acid (HC58-107). White solid, yield 61%. ESI m/z=591.2[M+H⁺].

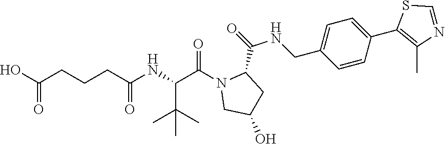 [SMILES: Cc1ncsc1-c1ccc(CNC(=O)[C@@H]2C[C@H](O)CN2C(=O)[C@@H](NC(=O)CCCC(=O)O)C(C)(C)C)cc1]

5-(((S)-1-((2S,4S)-4-Hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (HC58-108). White solid, yield 65%. ESI m/z=545.2[M+H⁺].

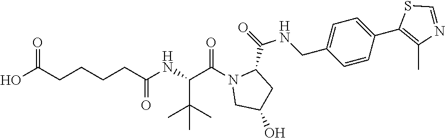 [SMILES: Cc1ncsc1-c1ccc(CNC(=O)[C@@H]2C[C@H](O)CN2C(=O)[C@@H](NC(=O)CCCCC(=O)O)C(C)(C)C)cc1]

6-(((S)-1-((2S,4S)-4-Hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (HC58-109). White solid, yield 65%. ESI m/z=559.3[M+H⁺].

Other Linker-EL moieties were synthesized following the published procedures described in WO 2018/106870 A1, the disclosure of which is hereby incorporated by reference.

### Example 3. Synthesis of ALK Degraders

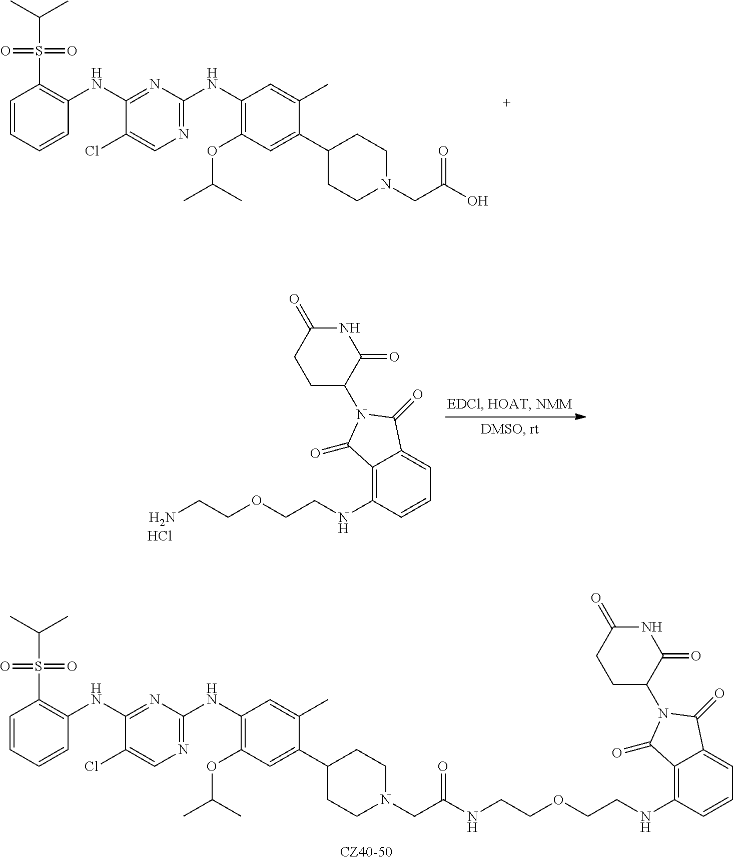 [SMILES: Cc1cc(Nc2ncc(Cl)c(Nc3ccccc3S(=O)(=O)C(C)C)n2)c(OC(C)C)cc1C1CCN(CC(=O)O)CC1 ; NCCOCCNc1cccc2c1C(=O)N(C1CCC(=O)NC1=O)C2=O ; Cl ; Cc1cc(Nc2ncc(Cl)c(Nc3ccccc3S(=O)(=O)C(C)C)n2)c(OC(C)C)cc1C1CCN(CC(=O)NCCOCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(2-(2-((2-(2,6-dioxopiperidin-3-yl)-1,3-dioxoisoindolin-4-yl)amino)ethoxy)ethyl)acetamide (CZ40-50). To a solution of 2-(4-(4-((5-chloro-4-((2-(isopropylsulfonyl)phenyl)amino)pyrimidin-2-yl)amino)-5-isopropoxy-2-methylphenyl)piperidin-1-yl)acetic acid (12 mg, 0.02 mmol) and 4-((2-(2-aminoethoxy)ethyl)amino)-2-(2,6-dioxopiperidin-3-yl)isoindoline-1,3-dione (8.6 mg, 0.02 mmol) in DMSO (1.0 mL) were added NMM (0.011 mL, 0.1 mmol), EDCI (6 mg, 0.03 mmol) and HOAT (4 mg, 0.03 mmol). The reaction mixture was stirred at room temperature overnight and purified by prepared HPLC to give the desired product CZ40-50 as yellow solid (10 mg, yield 52%). ¹H NMR (600 MHz, CD₃OD) δ 8.44 (d, J=8.4 Hz, 1H), 8.18 (s, 1H), 7.95 (d, J=7.8 Hz, 1H), 7.79 (s, 1H), 7.70 (t, J=7.8 Hz, 1H), 7.57 (t, J=7.8 Hz, 1H), 7.41 (t, J=7.8 Hz, 1H), 7.09 (d, J=8.4 Hz, 1H), 7.06 (d, J=6.6 Hz, 1H), 6.80 (s, 1H), 5.09 (dd, J=12.6, 5.4 Hz, 1H), 4.60-4.56 (m, 1H), 3.93-3.86 (m, 2H), 3.74-3.42 (m, 10H), 3.36-3.30 (m, 1H), 3.23-3.12 (m, 2H), 3.07-3.03 (m, 1H), 2.90-2.84 (m, 1H), 2.78-2.69 (m, 1H), 2.18-1.85 (m, 9H), 1.33 (d, J=6.0 Hz, 6H), 1.25 (d, J=6.6 Hz, 6H). HRMS calcd for C₄₇H₅₇ClN₉O₉S [M+H⁺]958.3683, found 958.3684.

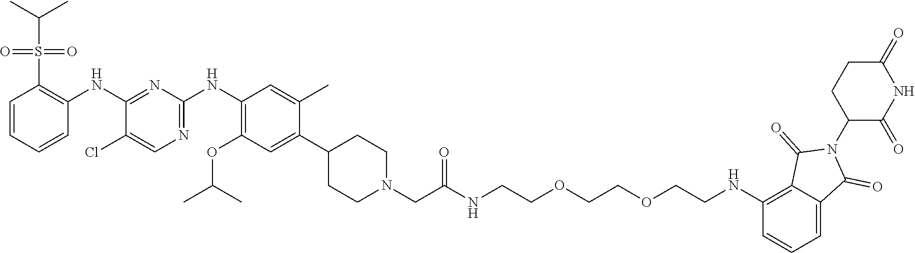 [SMILES: Cc1cc(Nc2ncc(Cl)c(Nc3ccccc3S(=O)(=O)C(C)C)n2)c(OC(C)C)cc1C1CCN(CC(=O)NCCOCCOCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(2-(2-(2-((2-(2,6-dioxopiperidin-3-yl)-1,3-dioxoisoindolin-4-yl)amino)ethoxy)ethoxy)ethyl)acetamide (CZ40-51). The title compound (yield 60%) was synthesized using the same procedure for the preparation of CZ40-50 as yellow solid. ¹H NMR (600 MHz, CD₃OD) δ 8.40 (d, J=8.4 Hz, 1H), 8.19 (s, 1H), 7.95 (d, J=7.8 Hz, 1H), 7.71 (s, 1H), 7.69 (t, J=8.4 Hz, 1H), 7.55 (t, J=7.8 Hz, 1H), 7.41 (t, J=7.8 Hz, 1H), 7.10 (d, J=9.0 Hz, 1H), 7.06 (d, J=7.2 Hz, 1H), 6.84 (s, 1H), 5.07 (dd, J=12.8, 5.4 Hz, 1H), 4.62-4.57 (m, 1H), 3.90 (s, 2H), 3.73 (t, J=4.8 Hz, 2H), 3.68-3.63 (m, 6H), 3.61 (t, J=5.4 Hz, 2H), 3.51 (t, J=5.4 Hz, 2H), 3.45 (t, J=5.4 Hz, 2H), 3.36-3.30 (m, 1H), 3.21 (t, J=12.0 Hz, 2H), 3.08-3.04 (m, 1H), 2.89-2.82 (m, 1H), 2.77-2.68 (m, 1H), 2.14-1.97 (m, 9H), 1.33 (d, J=6.0 Hz, 6H), 1.25 (d, J=6.6 Hz, 6H). HRMS calcd for C₄₉H₆₁ClN₉O₁₀S [M+H⁺] 1002.3945, found 1002.3920.

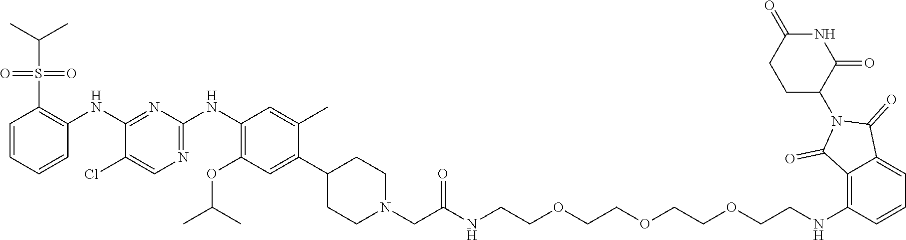 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(2-(2-(2-(2-((2-(2,6-dioxopiperidin-3-yl)-1,3-dioxoisoindolin-4-yl)amino)ethoxy)ethoxy)ethoxy)ethyl)acetamide (CZ40-52). The title compound (yield 69%) was synthesized using the same procedure for the preparation of CZ40-50 as yellow solid. ¹H NMR (600 MHz, CD₃OD) δ 8.42 (d, J=8.4 Hz, 1H), 8.18 (s, 1H), 7.94 (d, J=7.8 Hz, 1H), 7.77 (s, 1H), 7.69 (t, J=7.8 Hz, 1H), 7.54 (t, J=7.8 Hz, 1H), 7.39 (t, J=7.8 Hz, 1H), 7.08 (d, J=8.4 Hz, 1H), 7.05 (d, J=6.6 Hz, 1H), 6.82 (s, 1H), 5.06 (dd, J=12.6, 5.4 Hz, 1H), 4.61-4.56 (m, 1H), 3.92 (s, 2H), 3.72 (t, J=5.4 Hz, 2H), 3.69-3.65 (m, 8H), 3.62-3.60 (m, 2H), 3.57 (t, J=5.4 Hz, 2H), 3.49 (t, J=5.4 Hz, 2H), 3.44 (t, J=5.4 Hz, 2H), 3.35-3.30 (m, 1H), 3.20 (t, J=12.0 Hz, 2H), 3.08-3.04 (m, 1H), 2.89-2.83 (m, 1H), 2.77-2.68 (m, 1H), 2.14-1.97 (m, 9H), 1.33 (d, J=6.0 Hz, 6H), 1.25 (d, J=6.6 Hz, 6H). HRMS calcd for C₅₁H₆₅ClN₉O₁₁S [M+H⁺] 1046.4207, found 1046.4222.

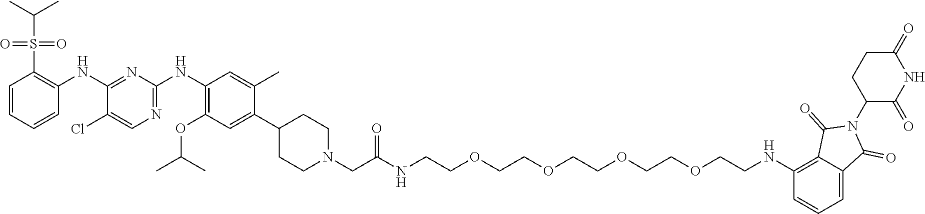 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(14-((2-(2,6-dioxopiperidin-3-yl)-1,3-dioxoisoindolin-4-yl)amino)-3,6,9,12-tetraoxatetradecyl)acetamide (CZ40-53). The title compound (yield 53%) was synthesized using the same procedure for the preparation of CZ40-50 as yellow solid. ¹H NMR (600 MHz, CD₃OD) δ 8.38 (d, J=8.4 Hz, 1H), 8.19 (s: 1H), 7.94 (d, J=7.8 Hz, 1H), 7.69 (t, J=7.8 Hz, 1H), 7.67 (s, 1H), 7.54 (t, J=7.8 Hz, 1H), 7.42 (t, J=7.8 Hz, 1H), 7.07 (d, J=8.4 Hz, 1H), 7.05 (d, J=12 Hz, 1H), 6.84 (s, 1H), 5.06 (dd, J=12.6, 5.4 Hz, 1H), 4.61-4.56 (m, 1H), 3.95 (s, 2H), 3.73-3.56 (m, 18H), 3.47 (t, J=5.4 Hz, 2H), 3.45 (t, J=5.4 Hz, 2H), 3.36-3.30 (m, 1H), 3.22 (t, J=12.0 Hz, 2H), 3.08-3.04 (m, 1H), 2.89-2.83 (m, 1H), 2.76-2.68 (m, 1H), 2.17-1.97 (m, 9H), 1.32 (d, J=6.0 Hz, 6H), 1.25 (d, J=6.6 Hz, 6H). HRMS calcd for C₅₃H₆₉ClN₉O₁₂S [M+H⁺] 1090.4469, found 1090.4472.

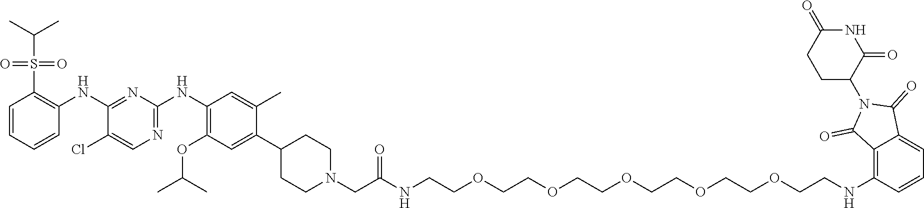 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCOCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(17-((2-(2,6-dioxopiperidin-3-yl)-1,3-dioxoisoindolin-4-yl)amino)-3,6,9,12,15-pentaoxaheptadecyl)acetamide (CZ40-77). The title compound (yield 59%) was synthesized using the same procedure for the preparation of CZ40-50 as yellow solid. ¹H NMR (600 MHz, CD₃OD) δ 8.36 (d, J=7.8 Hz, 1H), 8.19 (s, 1H), 7.95 (d, J=7.8 Hz, 1H), 7.69 (t, J=7.8 Hz, 1H), 7.63 (s, 1H), 7.53 (t, J=7.8 Hz, 1H), 7.43 (t, J=7.8 Hz, 1H), 7.06 (d, J=8.4 Hz, 1H), 7.04 (d, J=12 Hz, 1H), 6.85 (s, 1H), 5.05 (dd, J=12.6, 5.4 Hz, 1H), 4.61-4.56 (m, 1H), 3.96 (s, 2H), 3.72-3.61 (m, 20H), 3.58 (t, J=5.4 Hz, 2H), 3.48-3.44 (m, 4H), 3.37-3.30 (m, 1H), 3.24 (t, J=12.0 Hz, 2H), 3.09-3.05 (m, 1H), 2.88-2.82 (m, 1H), 2.75-2.66 (m, 1H), 2.14-1.97 (m, 9H), 1.32 (d, J=6.0 Hz, 6H), 1.24 (d, J=7.2 Hz, 6H). ¹³C NMR (150 MHz, CD₃OD) δ 173.2, 170.2, 169.3, 167.8, 163.9, 157.3, 155.7, 153.9, 146.7, 145.8, 137.9, 136.0, 135.9, 134.7, 132.4, 131.0, 127.6, 127.3, 125.8, 124.4, 123.8, 122.3, 116.9, 110.9, 110.7, 109.8, 105.2, 71.6, 70.2, 70.1, 70.0, 69.8, 69.1, 68.7, 57.3, 55.4, 54.0, 48.8, 41.8, 39.1, 34.8, 30.8, 29.6, 22.4, 21.0, 17.7, 14.1. HRMS calcd for C₅₅H₇₃ClN₉O₁₃S [M+H⁺] 1134.4732, found 1134.4739.

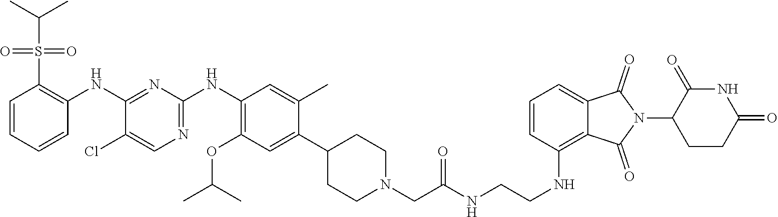 [SMILES: Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(2-((2-(2,6-dioxopiperidin-3-yl)-1,3-dioxoisoindolin-4-yl)amino)ethyl)acetamide (CZ40-78). The title compound (yield 80%) was synthesized using the same procedure for the preparation of CZ40-50 as yellow solid. ¹H NMR (600 MHz, CD₃OD) δ 8.38 (d, J=7.8 Hz, 1H), 8.19 (s, 1H), 7.95 (d, J=7.8 Hz, 1H), 7.70 (t, J=7.2 Hz, 1H), 7.67 (s, 1H), 7.58 (t, J=7.8 Hz, 1H), 7.43 (t, J=7.8 Hz, 1H), 7.16 (d, J=8.4 Hz, 1H), 7.08 (d, J=12 Hz, 1H), 6.85 (s, 1H), 5.03 (dd, J=12.6, 5.4 Hz, 1H), 4.61-4.56 (m, 1H), 3.92 (s, 2H), 3.64 (d, J=10.2 Hz, 2H), 3.59-3.54 (m, 4H), 3.37-3.31 (m, 1H), 3.19 (t, J=12.0 Hz, 2H), 3.09-3.05 (m, 1H), 2.83-2.77 (m, 1H), 2.70-2.64 (m, 1H), 2.16 (s, 3H), 2.07-1.97 (m, 6H), 1.33 (d, J=6.0 Hz, 6H), 1.24 (d, J=7.2 Hz, 6H). ¹³C NMR (150 MHz, CD₃OD) δ 173.0, 170.1, 169.3, 167.6, 164.5, 157.3, 155.7, 154.0, 146.6, 145.8, 137.9, 136.0, 135.9, 134.6, 132.5, 131.0, 127.6, 127.3, 125.7, 124.3, 123.8, 122.3, 116.7, 110.87, 110.85, 110.1, 105.1, 71.6, 57.2, 55.3, 53.8, 48.7, 41.3, 38.2, 34.7, 30.7, 29.5, 22.3, 21.0, 17.7, 14.0. HRMS calcd for C₄₅H₅₃ClN₉O₈S [M+H⁺] 914.3421, found 914.3434.

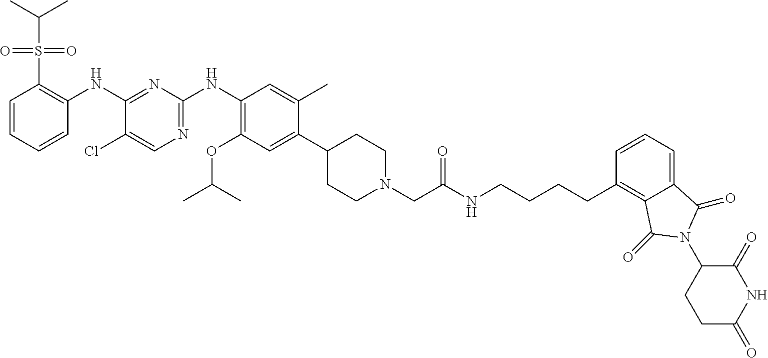 [SMILES: Cc1cc(Nc2ncc(Cl)c(Nc3ccccc3S(=O)(=O)C(C)C)n2)c(OC(C)C)cc1C1CCN(CC(=O)NCCCCc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(3-((2-(2,6-dioxopiperidin-3-yl)-1,3-dioxoisoindolin-4-yl)amino)propyl)acetamide (CZ40-79). The title compound (yield 72%) was synthesized using the same procedure for the preparation of CZ40-50 as yellow solid. ¹H NMR (600 MHz, CD₃OD) δ 8.37 (d, J=8.4 Hz, 1H), 8.19 (s, 1H), 7.95 (d, J=8.4 Hz, 1H), 7.70 (t, J=7.2 Hz, 1H), 7.65 (s, 1H), 7.56 (t, J=7.8 Hz, 1H), 7.44 (t, J=7.8 Hz, 1H), 7.07 (d, J=8.4 Hz, 1H), 7.05 (d, J=7.2 Hz, 1H), 6.84 (s, 1H), 5.08 (dd, J=12.6, 5.4 Hz, 1H), 4.62-4.58 (m, 1H), 3.93 (s, 2H), 3.64 (brs, 2H), 3.44 (t, J=6.6 Hz, 4H), 3.37-3.31 (m, 1H), 3.25-3.19 (m, 2H), 3.11-3.07 (m, 1H), 2.89-2.84 (m, 1H), 2.76-2.68 (m, 1H), 2.17-1.90 (m, 11H), 1.32 (d, J=6.0 Hz, 6H), 1.24 (d, J=12 Hz, 6H). HRMS calcd for C₄₆H₅₅ClN₉O₈S [M+H⁺] 928.3577, found 928.3555.

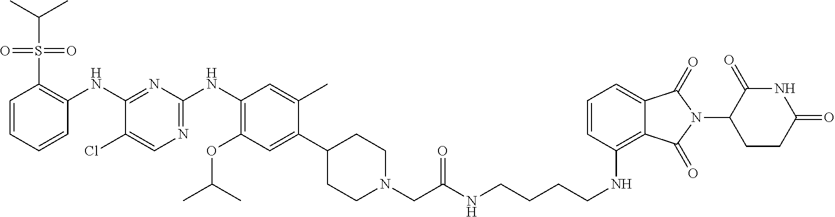 [SMILES: Cc1cc(Nc2ncc(Cl)c(Nc3ccccc3S(=O)(=O)C(C)C)n2)c(OC(C)C)cc1C1CCN(CC(=O)NCCCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(4-((2-(2,6-dioxopiperidin-3-yl)-1,3-dioxoisoindolin-4-yl)amino)butyl)acetamide (CZ40-80). The title compound (yield 71%) was synthesized using the same procedure for the preparation of CZ40-50 as yellow solid. ¹H NMR (600 MHz, CD₃OD) δ 8.37 (d, J=8.4 Hz, 1H), 8.19 (s, 1H), 7.95 (d, J=8.4 Hz, 1H), 7.70 (t, J=12 Hz, 1H), 7.65 (s, 1H), 7.55 (t, J=7.8 Hz, 1H), 7.43 (t, J=12 Hz, 1H), 7.06 (d, J=9.0 Hz, 1H), 7.04 (d, J=12 Hz, 1H), 6.86 (s, 1H), 5.02 (dd, J=12.6, 5.4 Hz, 1H), 4.62-4.58 (m, 1H), 3.94 (s, 2H), 3.68 (d, J=11.4 Hz, 2H), 3.39-3.33 (m, 5H), 3.23 (t, J=11.4 Hz, 2H), 3.11-3.07 (m, 1H), 2.85-2.79 (m, 1H), 2.73-2.64 (m, 1H), 2.17 (s, 3H), 2.08-1.98 (m, 6H), 1.73-1.66 (m, 4H), 1.32 (d, J=6.0 Hz, 6H), 1.24 (d, J=12 Hz, 6H). HRMS calcd for C₄₇H₅₇ClN₉O₈S [M+H⁺] 942.3734, found 942.3721.

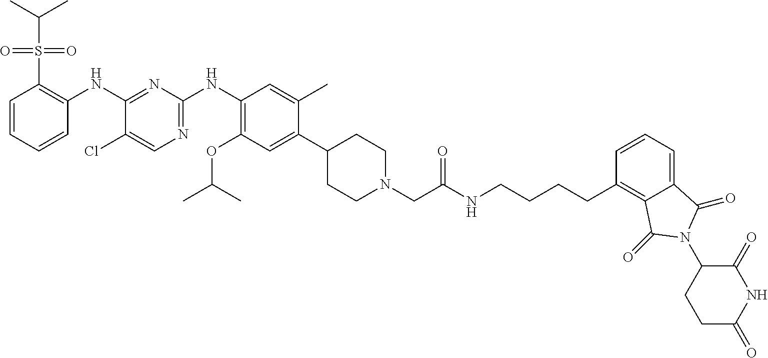 [SMILES: Cc1cc(Nc2ncc(Cl)c(Nc3ccccc3S(=O)(=O)C(C)C)n2)c(OC(C)C)cc1C1CCN(CC(=O)NCCCCc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(5-((2-(2,6-dioxopiperidin-3-yl)-1,3-dioxoisoindolin-4-yl)amino)pentyl)acetamide (CZ40-81). The title compound (yield 84%) was synthesized using the same procedure for the preparation of CZ40-50 as yellow solid. ¹H NMR (600 MHz, CD₃OD) δ 8.38 (d, J=7.8 Hz, 1H), 8.19 (s, 1H), 7.95 (d, J=8.4 Hz, 1H), 7.71 (t, J=12 Hz, 1H), 7.67 (s, 1H), 7.54 (t, J=7.8 Hz, 1H), 7.43 (t, J=12 Hz, 1H), 7.05 (d, J=8.4 Hz, 1H), 7.02 (d, J=6.6 Hz, 1H), 6.84 (s, 1H), 5.05 (dd, J=12.6, 5.4 Hz, 1H), 4.62-4.58 (m, 1H), 3.92 (s, 2H), 3.64 (d, J=11.4 Hz, 2H), 3.36-3.30 (m, 5H), 3.22 (t, J=12.0 Hz, 2H), 3.09-3.05 (m, 1H), 2.87-2.81 (m, 1H), 2.75-2.67 (m: 1H), 2.16 (s, 3H), 2.10-1.94 (m, 6H), 1.73-1.68 (m, 2H), 1.64-1.59 (m, 2H), 1.51-1.46 (m, 2H), 1.32 (d, J=6.0 Hz, 6H), 1.24 (d, J=7.2 Hz, 6H). HRMS calcd for C₄₈H₅₉ClN₉O₈S [M+H⁺] 956.3890, found 956.3882.

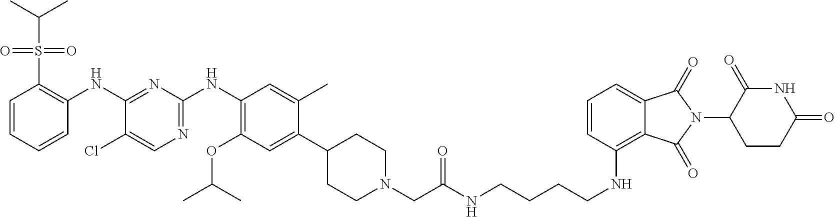 [SMILES: Cc1cc(Nc2ncc(Cl)c(Nc3ccccc3S(=O)(=O)C(C)C)n2)c(OC(C)C)cc1C1CCN(CC(=O)NCCCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(6-((2-(2,6-dioxopiperidin-3-yl)-1,3-dioxoisoindolin-4-yl)amino)hexyl)acetamide (CZ40-82). The title compound (yield 89%) was synthesized using the same procedure for the preparation of CZ40-50 as yellow solid. ¹H NMR (600 MHz, CD₃OD) δ 8.38 (d, J=7.8 Hz, 1H), 8.19 (s, 1H), 7.95 (d, J=8.4 Hz, 1H), 7.70 (t, J=7.2 Hz, 1H), 7.67 (s, 1H), 7.54 (t, J=7.8 Hz, 1H), 7.42 (t, J=7.2 Hz, 1H), 7.04 (d, J=8.4 Hz, 1H), 7.02 (d, J=6.6 Hz, 1H), 6.85 (s, 1H), 5.04 (dd, J=12.6, 5.4 Hz, 1H), 4.62-4.58 (m, 1H), 3.93 (s, 2H), 3.68 (d, J=11.4 Hz, 2H), 3.37-3.21 (m, 7H), 3.10-3.06 (m, 1H), 2.87-2.81 (m, 1H), 2.75-2.67 (m, 1H), 2.16 (s, 3H), 2.11-1.98 (m, 6H), 1.70-1.66 (m, 2H), 1.60-1.55 (m, 2H), 1.50-1.42 (m, 4H), 1.32 (d, J=6.0 Hz, 6H), 1.24 (d, J=7.2 Hz, 6H). HRMS calcd for C₄₉H₆₁ClN₉O₈S [M+H⁺] 970.4047, found 970.4033.

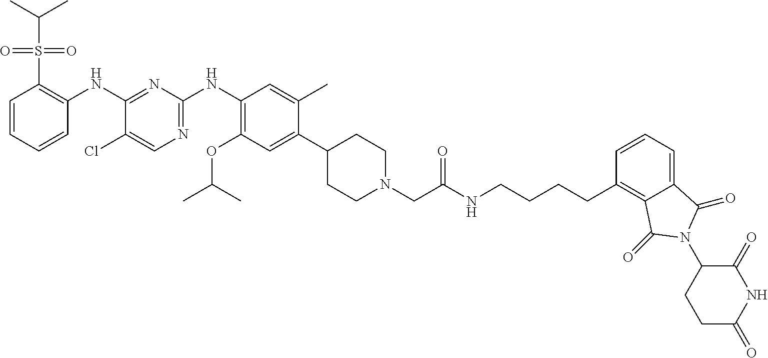 [SMILES: Cc1cc(Nc2ncc(Cl)c(Nc3ccccc3S(=O)(=O)C(C)C)n2)c(OC(C)C)cc1C1CCN(CC(=O)NCCCCc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(7-((2-(2,6-dioxopiperidin-3-yl)-1,3-dioxoisoindolin-4-yl)amino)heptyl)acetamide (CZ40-83). The title compound (yield 51%) was synthesized using the same procedure for the preparation of CZ40-50 as yellow solid. ¹H NMR (600 MHz, CD₃OD) δ 8.43 (d, J=8.4 Hz, 1H), 8.18 (s, 1H), 7.94 (d, J=7.8 Hz, 1H), 7.78 (s, 1H), 7.68 (t, J=7.2 Hz, 1H), 7.54 (t, J=7.8 Hz, 1H), 7.39 (t, J=7.2 Hz, 1H), 7.04 (d, J=8.4 Hz, 1H), 7.02 (d, J=6.6 Hz, 1H), 6.84 (s, 1H), 5.04 (dd, J=12.6, 5.4 Hz, 1H), 4.62-4.58 (m, 1H), 3.92 (s, 2H), 3.68 (d, J=11.4 Hz, 2H), 3.36-3.21 (m, 7H), 3.10-3.06 (m, 1H), 2.87-2.81 (m, 1H), 2.75-2.66 (m, 1H), 2.16 (s, 3H), 2.11-1.99 (m, 6H), 1.70-1.65 (m, 2H), 1.58-1.54 (m, 2H), 1.45-1.38 (m, 6H), 1.33 (d, J=6.0 Hz, 6H), 1.25 (d, J=12 Hz, 6H). HRMS calcd for C₅₀H₆₃ClN₉O₈S [M+H⁺] 984.4203, found 984.4211.

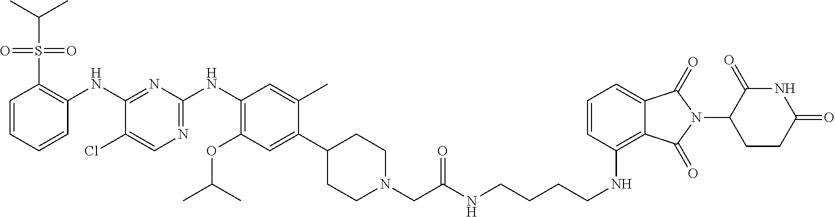 [SMILES: Cc1cc(Nc2ncc(Cl)c(Nc3ccccc3S(=O)(=O)C(C)C)n2)c(OC(C)C)cc1C1CCN(CC(=O)NCCCCNc2cccc3c2C(=O)N(C2CCC(=O)NC2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(8-((2-(2,6-dioxopiperidin-3-yl)-1,3-dioxoisoindolin-4-yl)amino)octyl)acetamide (CZ40-84). The title compound (yield 67%) was synthesized using the same procedure for the preparation of CZ40-50 as yellow solid. ¹H NMR (600 MHz, CD₃OD) δ 8.38 (d, J=8.4 Hz, 1H), 8.18 (s, 1H), 7.94 (d, J=7.8 Hz, 1H), 7.70-7.67 (m, 1H), 7.53 (t, J=7.8 Hz, 1H), 7.41 (t, J=12 Hz, 1H), 7.03 (d, J=8.4 Hz, 1H), 7.01 (d, J=6.6 Hz, 1H), 6.85 (s, 1H), 5.04 (dd, J=12.6, 5.4 Hz, 1H), 4.62-4.58 (m, 1H), 3.93 (s, 2H), 3.69 (d, J=11.4 Hz, 2H), 3.36-3.21 (m, 7H), 3.10-3.06 (m, 1H), 2.87-2.81 (m, 1H), 2.75-2.66 (m, 1H), 2.16 (s, 3H), 2.11-1.99 (m, 6H), 1.69-1.64 (m, 2H), 1.58-1.54 (m, 2H), 1.45-1.37 (m, 8H), 1.33 (d, J=6.0 Hz, 6H), 1.25 (d, J=12 Hz, 6H). HRMS calcd for C₅₁H₆₅ClN₉O₈S [M+H⁺] 998.4360, found 998.4348.

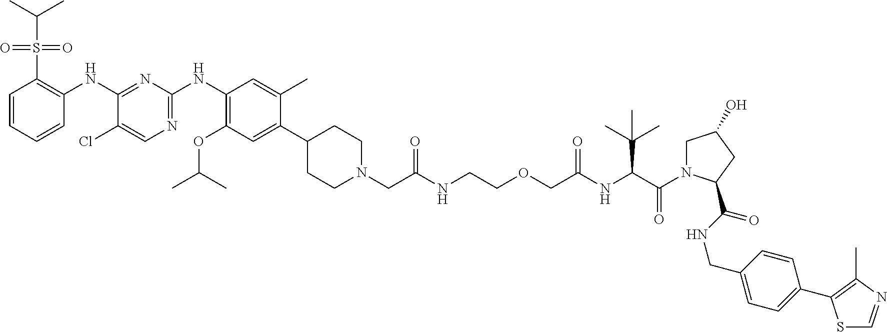 [SMILES: Cc1cc(Nc2ncc(Cl)c(Nc3ccccc3S(=O)(=O)C(C)C)n2)c(OC(C)C)cc1C1CCN(CC(=O)NCCOCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(2-(2-(2-(4-(4-((5-Chloro-4-((2-(isopropylsulfonyl)phenyl)amino)pyrimidin-2-yl)amino)-5-isopropoxy-2-methylphenyl)piperidin-1-yl)acetamido)ethoxy)acetamido)-3,3-dimethylbutanoyl)-4-hydroxy-N-(4-(4-methylthiazol-5-yl)benzyl)pyrrolidine-2-carboxamide (CZ40-85). The title compound (yield 48%) was synthesized using the same procedure for the preparation of CZ40-50 as white solid. ¹H NMR (600 MHz, CD₃OD) δ 8.89 (s, 1H), 8.26 (d, J=7.8 Hz, 1H), 8.20 (s, 1H), 7.95 (dd, J=7.8, 1.2 Hz, 1H), 7.68 (t, J=7.8 Hz, 1H), 7.64 (s, 1H), 7.42 (t, J=7.8 Hz, 1H), 7.33 (d, J=7.8 Hz, 2H), 7.27 (d, J=1.9 Hz, 2H), 6.87 (s, 1H), 4.78 (d, J=9.6 Hz, 1H), 4.70-4.66 (m, 1H), 4.58-4.52 (m, 1H), 4.29 (d, J=15.0 Hz, 1H), 4.12-3.89 (m, 6H), 3.82-3.55 (m, 8H), 3.41 (d, J=14.4 Hz, 1H), 3.25-3.22 (m, 1H), 3.10-3.08 (m, 1H), 2.42 (s, 3H), 2.29-2.25 (m, 1H), 2.16 (s, 3H), 2.12-1.98 (m, 6H), 1.32 (d, J=6.0 Hz, 6H), 1.22 (d, J=7.2 Hz, 6H), 1.09 (s, 9H). HRMS calcd for C₅₆H₇₄ClN₁₀O₉S₂ [M+H⁺] 1129.4765, found 1129.4757.

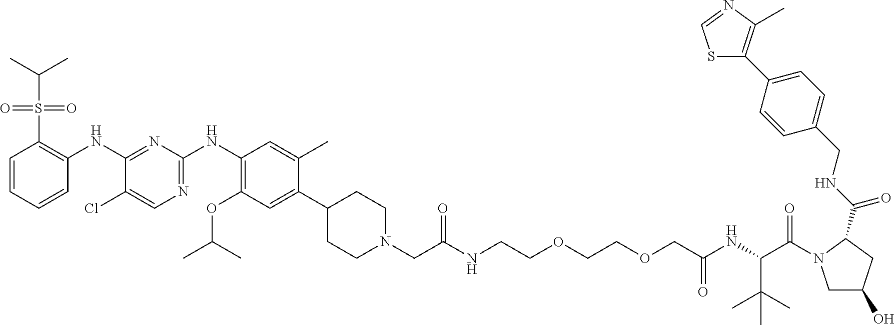 [SMILES: Cc1cc(Nc2ncc(Cl)c(Nc3ccccc3S(=O)(=O)C(C)C)n2)c(OC(C)C)cc1C1CCN(CC(=O)NCCOCCOCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(tert-Butyl)-14-(4-(4-((5-chloro-4-((2-(isopropylsulfonyl)phenyl)amino)pyrimidin-2-yl)amino)-5-isopropoxy-2-methylphenyl)piperidin-1-yl)-4,13-dioxo-6,9-dioxa-3,12-diazatetradecanoyl)-4-hydroxy-N-(4-(4-methylthiazol-5-yl)benzyl)pyrrolidine-2-carboxamide (CZ40-86). The title compound (yield 51%) was synthesized using the same procedure for the preparation of CZ40-50 as white solid. ¹H NMR (600 MHz, CD₃OD) δ 8.89 (s, 1H), 8.35 (d, J=7.8 Hz, 1H), 8.20 (s, 1H), 7.95 (dd, J=7.8, 1.2 Hz, 1H), 7.68 (t, J=7.8 Hz, 1H), 7.61 (s, 1H), 7.44-7.39 (m, 5H), 6.85 (s, 1H), 4.78 (d, J=9.6 Hz, 1H), 4.64-4.41 (m, 5H), 4.07-3.96 (m, 4H), 3.91-3.86 (m, 2H), 3.72-3.57 (m, 10H), 3.37-3.32 (m, 1H), 3.23-3.19 (m, 1H), 3.10-3.07 (m, 1H), 2.46 (s, 3H), 2.32-2.28 (m, 1H), 2.16 (s, 3H), 2.13-1.94 (m, 6H), 1.32 (d, J=6.0 Hz, 6H), 1.24 (d, J=7.2 Hz, 6H), 1.05 (s, 9H). HRMS calcd for C₅₈H₇₈ClN₁₀O₁₀S₂ [M+H⁺] 1173.5027, found 1173.5016.

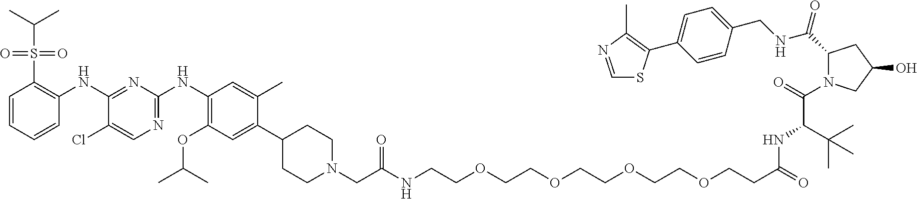 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-20-(tert-Butyl)-1-(4-(4-((5-chloro-4-((2-(isopropylsulfonyl)phenyl)amino)pyrimidin-2-yl)amino)-5-isopropoxy-2-methylphenyl)piperidin-1-yl)-2,18-dioxo-6,9,12,15-tetraoxa-3,19-diazahenicosan-21-oyl)-4-hydroxy-7V-(4-(4-methylthiazol-5-yl)benzyl)pyrrolidine-2-carboxamide (CZ40-47). The title compound (yield 68%) was synthesized using the same procedure for the preparation of CZ40-50 as white solid. ¹H NMR (600 MHz, CD₃OD) δ 8.91 (s, 1H), 8.33 (d, J=7.8 Hz, 1H), 8.20 (s, 1H), 7.96 (dd, J=7.8, 1.2 Hz, 1H), 7.69 (t, J=7.8 Hz, 1H), 7.57 (s, 1H), 7.47-7.39 (m, 5H), 6.88 (s, 1H), 4.66-4.48 (m, 5H), 4.36 (d, J=15.0 Hz, 1H), 3.97 (s, 2H), 3.88 (d, J=10.8 Hz, 1H), 3.79 (dd, J=10.8, 4.8 Hz, 1H), 3.74-3.72 (m, 4H), 3.63 (brs, 12H), 3.59 (t, J=5.4 Hz, 2H), 3.46 (t, J=5.4 Hz, 2H), 3.37-3.33 (m, 1H), 3.28-3.24 (m, 1H), 3.12-3.09 (m, 1H), 2.60-2.55 (m, 1H), 2.49-2.46 (m, 4H), 2.23-2.20 (m, 1H), 2.17 (s, 3H), 2.09-2.00 (m, 6H), 1.32 (d, J=6.0 Hz, 6H), 1.24 (d, J=7.2 Hz, 6H), 1.03 (s, 9H). HRMS calcd for C₆₃H₈₈ClN₁₀O₁₂S₂ [M+H⁺] 1275.5708, found 1275.5706.

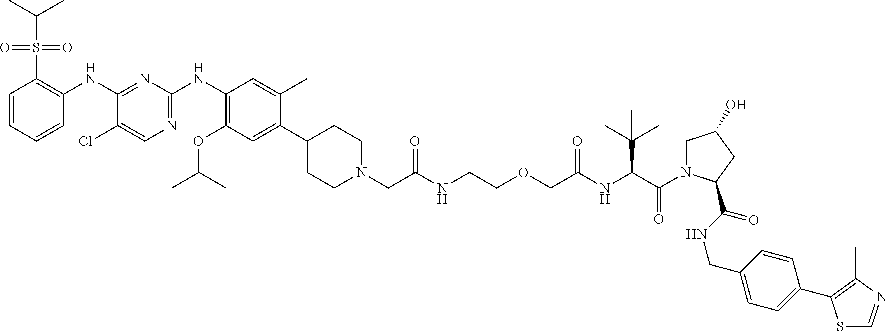 [SMILES: Cc1cc(Nc2ncc(Cl)c(Nc3ccccc3S(=O)(=O)C(C)C)n2)c(OC(C)C)cc1C1CCN(CC(=O)NCCOCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(5-(2-(4-(4-((5-Chloro-4-((2-(isopropylsulfonyl)phenyl)amino)pyrimidin-2-yl)amino)-5-isopropoxy-2-methylphenyl)piperidin-1-yl)acetamido)pentanamido)-3,3-dimethylbutanoyl)-4-hydroxy-N-(4-(4-methylthiazol-5-yl)benzyl)pyrrolidine-2-carboxamide (CZ40-88). The title compound (yield 53%) was synthesized using the same procedure for the preparation of CZ40-50 as white solid. ¹H NMR (600 MHz, CD₃OD) δ 8.89 (s, 1H), 8.34 (d, J=7.8 Hz, 1H), 8.20 (s, 1H), 7.96 (dd, J=7.8, 1.2 Hz, 1H), 7.69 (t, J=7.8 Hz, 1H), 7.65 (s, 1H), 7.44-7.36 (m, 5H), 6.87 (s, 1H), 4.68-4.64 (m, 2H), 4.56 (t, J=8.4 Hz, 1H), 4.48 (s, 1H), 4.37-4.29 (m, 2H), 4.01-3.94 (m, 2H), 3.89 (d, J=11.4 Hz, 1H), 3.81-3.71 (m, 3H), 3.40-3.32 (m, 2H), 3.28-3.24 (m, 2H), 3.12-3.09 (m, 1H), 2.45 (s, 3H), 2.40-2.21 (m, 3H), 2.17 (s, 3H), 2.10-1.99 (m, 6H), 1.67-1.56 (m, 4H), 1.32 (d, J=6.0 Hz, 6H), 1.24 (d, J=7.2 Hz, 6H), 1.06 (s, 9H). HRMS calcd for C₅₇H₇₆ClN₁₀O₈S₂ [M+H⁺] 1127.4972, found 1127.4956.

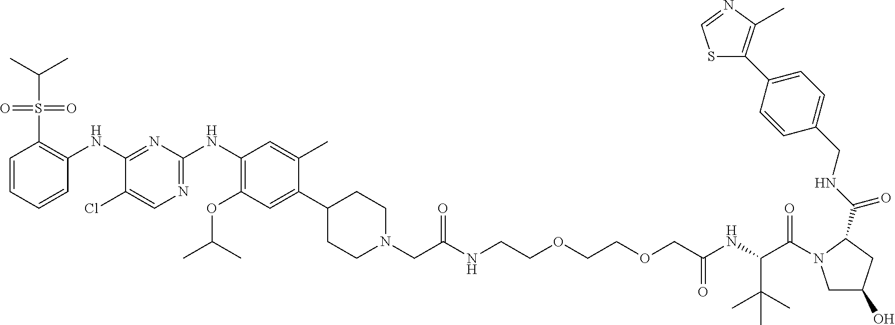 [SMILES: Cc1cc(Nc2ncc(Cl)c(Nc3ccccc3S(=O)(=O)C(C)C)n2)c(OC(C)C)cc1C1CCN(CC(=O)NCCOCCOCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(6-(2-(4-(4-((5-chloro-4-((2-(isopropylsulfonyl)phenyl)amino)pyrimidin-2-yl)amino)-5-isopropoxy-2-methylphenyl)piperidin-1-yl)acetamido)hexanamido)-3,3-dimethylbutanoyl)-4-hydroxy-N-(4-(4-methylthiazol-5-yl)benzyl)pyrrolidine-2-carboxamide (CZ40-89). The title compound (yield 58%) was synthesized using the same procedure for the preparation of CZ40-50 as white solid. ¹H NMR (600 MHz. Methanol-d₄) δ 8.89 (s, 1H), 8.36 (d, J=7.8 Hz, 1H), 8.20 (s, 1H), 7.96 (dd, J=7.8, 1.2 Hz, 1H), 7.69 (t, J=7.8 Hz, 1H), 7.62 (s, 1H), 7.46-7.39 (m, 5H), 6.87 (s, 1H), 4.65-4.50 (m, 5H), 4.36 (d, J=15.6 Hz, 1H), 3.94 (s, 2H), 3.89 (d, J=11.4 Hz, 1H), 3.80 (dd, J=11.4, 3.6 Hz, 1H), 3.70 (d, J=10.2 Hz, 2H), 3.37-3.33 (m, 1H), 3.28-3.24 (m, 3H), 3.12-3.08 (m, 1H), 2.46 (s, 3H), 2.32-2.20 (m, 3H), 2.17 (s, 3H), 2.10-2.00 (m, 6H), 1.65-1.54 (m, 4H), 1.38-1.36 (m, 2H), 1.32 (d, J=6.0 Hz, 6H), 1.24 (d, J=7.2 Hz, 6H), 1.03 (s, 9H). HRMS calcd for C₅₈H₇₈ClN₁₀O₈S₂ [M+H⁺] 1141.5129, found 1141.5128.

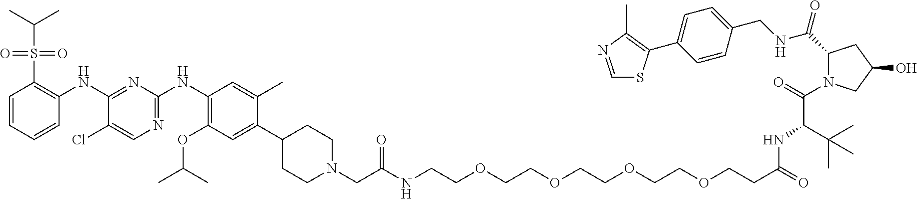 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(7-(2-(4-(4-((5-Chloro-4-((2-(isopropylsulfonyl)phenyl)amino)pyrimidin-2-yl)amino)-5-isopropoxy-2-methylphenyl)piperidin-1-yl)acetamido)heptanamido)-3,3-dimethylbutanoyl)-4-hydroxy-N-(4-(4-methylthiazol-5-yl)benzyl)pyrrolidine-2-carboxamide (CZ40-90). The title compound (yield 35%) was synthesized using the same procedure for the preparation of CZ40-50 as white solid. ¹H NMR (600 MHz, CD₃OD) δ 8.89 (s, 1H), 8.36 (d, J=7.8 Hz, 1H), 8.20 (s, 1H), 7.96 (dd, J=7.8, 1.2 Hz, 1H), 7.69 (t, J=7.8 Hz, 1H), 7.63 (s, 1H), 7.46-7.39 (m, 5H), 6.86 (s, 1H), 4.65-4.50 (m, 5H), 4.36 (d, J=15.6 Hz, 1H), 3.94 (s, 2H), 3.89 (d, J=11.4 Hz, 1H), 3.80 (dd, J=11.4, 3.6 Hz, 1H), 3.70 (d, J=10.2 Hz, 2H), 3.36-3.31 (m, 1H), 3.28-3.24 (m, 3H), 3.12-3.09 (m, 1H), 2.46 (s, 3H), 2.33-2.19 (m, 3H), 2.17 (s, 3H), 2.10-1.99 (m, 6H), 1.64-1.54 (m, 4H), 1.38-1.36 (m, 4H), 1.32 (d, J=6.0 Hz, 6H), 1.24 (d, J=7.2 Hz, 6H), 1.03 (s, 9H). HRMS calcd for C₅₉H₈₀ClN₁₀O₈S₂ [M+H⁺] 1155.5285, found 1155.5297.

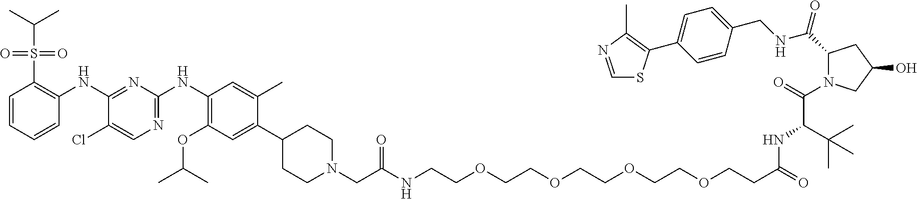 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(9-(2-(4-(4-((5-Chloro-4-((2-(isopropylsulfonyl)phenyl)amino)pyrimidin-2-yl)amino)-5-isopropoxy-2-methylphenyl)piperidin-1-yl)acetamido)nonanaimdo)-3,3-dimethylbutanoyl)-4-hydroxy-N-(4-(4-methylthiazol-5-yl)benzyl)pyrrolidine-2-carboxamide (CZ40-92). The title compound (yield 56%) was synthesized using the same procedure for the preparation of CZ40-50 as white solid. ¹H NMR (600 MHz, CD₃OD) δ 8.90 (s, 1H), 8.36 (d, J=7.8 Hz, 1H), 8.20 (s, 1H), 7.96 (dd, 7=7.8, 1.2 Hz, 1H), 7.69 (t, 7=7.8 Hz, 1H), 7.63 (s, 1H), 7.46-7.39 (m, 5H), 6.87 (s, 1H), 4.65-4.49 (m, 5H), 4.35 (d, 7=15.6 Hz, 1H), 3.94 (s, 2H), 3.89 (d, J=11.4 Hz, 1H), 3.80 (dd, J=11.4, 3.6 Hz, 1H), 3.70 (d, 7=10.2 Hz, 2H), 3.36-3.31 (m, 1H), 3.27-3.23 (m, 3H), 3.12-3.09 (m, 1H), 2.46 (s, 3H), 2.33-2.19 (m, 3H), 2.17 (s, 3H), 2.09-2.00 (m, 6H), 1.64-1.53 (m, 4H), 1.34 (brs, 8H), 1.32 (d, J=6.0 Hz, 6H), 1.24 (d, J=7.2 Hz, 6H), 1.03 (s, 9H). HRMS calcd for C₆₁N₈₄ClN₁₀O₈S₂ [M+H⁺] 1183.5598, found 1183.5584.

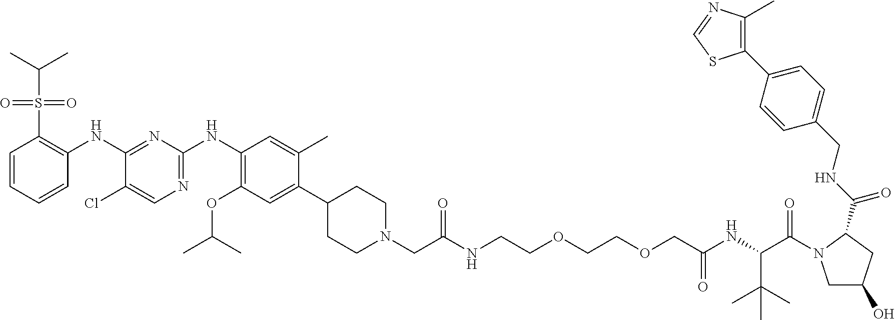 [SMILES: Cc1cc(Nc2ncc(Cl)c(Nc3ccccc3S(=O)(=O)C(C)C)n2)c(OC(C)C)cc1C1CCN(CC(=O)NCCOCCOCC(=O)N[C@H](C(=O)N2C[C@H](O)C[C@H]2C(=O)NCc2ccc(-c3scnc3C)cc2)C(C)(C)C)CC1]

(2S,4R)-1-((10-(2-(10-(2-(4-(4-((5-Chloro-4-((2-(isopropylsulfonyl)phenyl)amino)pyrimidin-2-yl)amino)-5-isopropoxy-2-methylphenyl)piperidin-1-yl)acetamido)decanamido)-3,3-dimethylbutanoyl)-4-hydroxy-N-(4-(4-methylthiazol-5-yl)benzyl)pyrrolidine-2-carboxamide (CZ40-93). The title compound (yield 17%) was synthesized using the same procedure for the preparation of CZ40-50 as white solid. ¹H NMR (600 MHz, CD₃OD) δ 8.88 (s, 1H), 8.41 (d, J=7.8 Hz, 1H), 8.18 (s, 1H), 7.94 (dd, 7=7.8, 1.2 Hz, 1H), 7.75 (s, 1H), 7.69 (t, J=7.8 Hz, 1H), 7.46-7.38 (m, 5H), 6.85 (s, 1H), 4.64-4.49 (m, 5H), 4.35 (d, J=15.6 Hz, 1H), 3.93 (s, 2H), 3.89 (d, J=11.4 Hz, 1H), 3.80 (dd, J=11.4, 3.6 Hz, 1H), 3.70 (d, J=10.2 Hz, 2H), 3.36-3.31 (m, 1H), 3.27-3.23 (m, 3H), 3.12-3.09 (m, 1H), 2.46 (s, 3H), 2.32-2.19 (m, 3H), 2.17 (s, 3H), 2.09-2.00 (m, 6H), 1.64-1.53 (m, 4H), 1.34 (d, J=6.0 Hz, 6H), 1.33 (brs, 10H), 1.25 (d, J=7.2 Hz, 6H), 1.03 (s, 9H). HRMS calcd for C₆₂H₈₆ClN₁₀O₈S₂ [M+H⁺] 1197.5755, found 1197.5750.

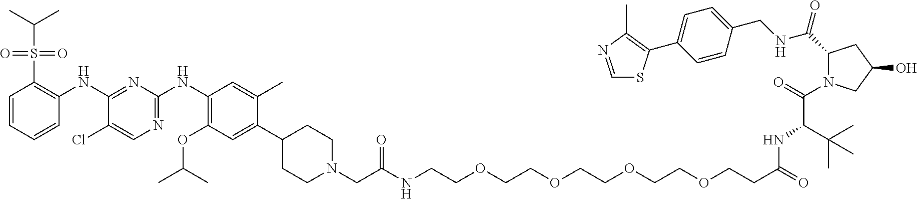 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-((11-(2-(4-(4-((5-Chloro-4-((2-(isopropylsulfonyl)phenyl)amino)pyrimidin-2-yl)amino)-5-isopropoxy-2-methylphenyl)piperidin-1-yl)acetamido)undecanamido)-3,3-dimethylbutanoyl)-4-hydroxy-N-(4-(4-methylthiazol-5-yl)benzyl)pyrrolidine-2-carboxamide (CZ40-94). The title compound (yield 73%) was synthesized using the same procedure for the preparation of CZ40-50 as white solid. ¹H NMR (600 MHz, CD₃OD) δ 8.88 (s, 1H), 8.39 (d, J=7.8 Hz, 1H), 8.18 (s, 1H), 7.94 (dd, J=7.8, 1.2 Hz, 1H), 7.69 (s, 1H), 7.67 (t, J=7.8 Hz, 1H), 7.46-7.39 (m, 5H), 6.86 (s, 1H), 4.64-4.49 (m, 5H), 4.35 (d, J=15.6 Hz, 1H), 3.94 (s, 2H), 3.89 (d, J=11.4 Hz, 1H), 3.80 (dd, J=11.4, 3.6 Hz, 1H), 3.70 (d, J=10.2 Hz, 2H), 3.36-3.31 (m, 1H), 3.27-3.23 (m, 3H), 3.12-3.09 (m, 1H), 2.46 (s, 3H), 2.32-2.19 (m, 3H), 2.17 (s, 3H), 2.09-2.00 (m, 6H), 1.64-1.53 (m, 4H), 1.33 (d, J=6.0 Hz, 6H), 1.31 (brs, 12H), 1.25 (d, J=7.2 Hz, 6H), 1.03 (s, 9H). HRMS calcd for C₆₃H₈₈ClN₁₀O₈S₂ [M+H⁺] 1211.5911, found 1211.5895.

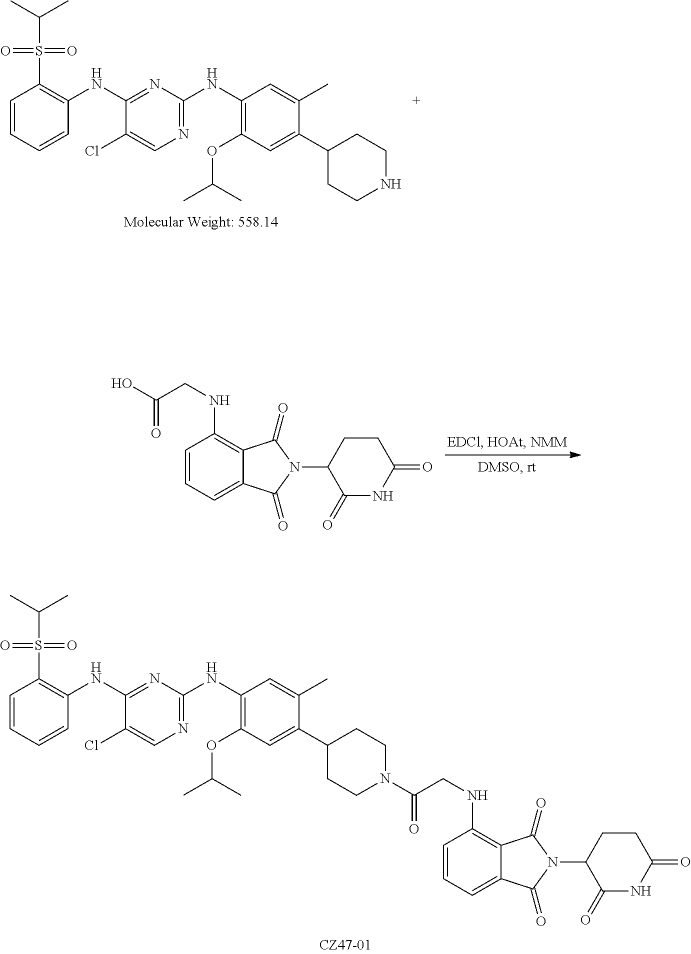 [SMILES: Cc1cc(Nc2ncc(Cl)c(Nc3ccccc3S(=O)(=O)C(C)C)n2)c(OC(C)C)cc1C1CCNCC1 ; O=C(O)CNc1cccc2c1C(=O)N(C1CCC(=O)NC1=O)C2=O ; Cc1cc(Nc2ncc(Cl)c(Nc3ccccc3S(=O)(=O)C(C)C)n2)c(OC(C)C)cc1C1CCN(C(=O)CNc2cccc3c2C(=O)N(C2CCC(=O)NC2=O)C3=O)CC1]

4-((2-(4-(4-((5-Chloro-4-((2-(isopropylsulfonyl)phenyl)amino)pyrimidin-2-yl)amino)-5-isopropoxy-2-methylphenyl)piperidin-1-yl)-2-oxoethyl)amino)-2-(2,6-dioxopiperidin-3-yl)isoindoline-1,3-dione (CZ47-01). To a solution of ceritinib (11 mg, 0.02 mmol) and (2-(2,6-dioxopiperidin-3-yl)-1,3-dioxoisoindolin-4-yl)glycine (6.6 mg, 0.02 mmol) in DMSO (1.0 mL) were added NMM (0.011 mL, 0.1 mmol), EDCI (6 mg, 0.03 mmol) and HOAT (4 mg, 0.03 mmol). The reaction mixture was stirred at room temperature overnight and purified by prepared HPLC to give the desired product CZ47-01 as yellow solid (9.1 mg, yield 52%). ¹H NMR (600 MHz, CD₃OD) δ 8.35 (d, J=7.8 Hz, 1H), 8.18 (s, 1H), 7.97 (d, J=7.8 Hz, 1H), 7.71 (t, J=7.8 Hz, 1H), 7.55 (q, J=7.8 Hz, 1H), 7.48 (t, J=7.2 Hz, 1H), 7.46 (s, 1H), 7.07 (t, J=6.0 Hz, 1H), 7.02 (dd, J=7.2, 3.6 Hz, 1H), 6.90 (d, J=11.4 Hz, 1H), 5.07 (dd, J=12.6, 4.8 Hz, 1H), 4.71 (d, J=12.6 Hz, 1H), 4.62-4.59 (m, 1H), 4.28 (dd, J=16.8, 7.2 Hz, 1H), 4.20 (d, J=16.8 Hz, 1H), 4.09 (d, J=13.2 Hz, 1H), 3.38-3.34 (m, 1H), 3.28 (t, J=12.6 Hz, 1H), 3.08 (t, J=12.0 Hz, 1H), 2.84 (d, J=15.6 Hz, 2H), 2.74 (d, J=15.6 Hz, 2H), 2.20 (s, 3H), 2.11 (brs, 1H), 1.87-1.76 (m, 3H), 1.70-1.63 (m, 1H), 1.26-1.24 (m, 12H). HRMS calcd for C₄₃H₄₈ClN₈O₈S [M+H⁺] 871.2999, found 871.2996.

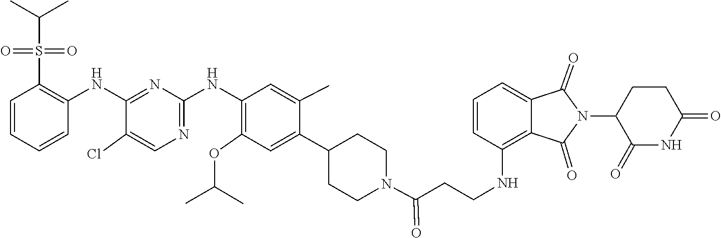 [SMILES: Cc1cc(Nc2ncc(Cl)c(Nc3ccccc3S(=O)(=O)C(C)C)n2)c(OC(C)C)cc1C1CCN(C(=O)CCNc2cccc3c2C(=O)N(C2CCC(=O)NC2=O)C3=O)CC1]

4-((3-(4-(4-((5-Chloro-4-((2-(isopropylsulfonyl)phenyl)amino)pyrimidin-2-yl)amino)-5-isopropoxy-2-methylphenyl)piperidin-1-yl)-3-oxopropyl)amino)-2-(2,6-dioxopiperidin-3-yl)isoindoline-1,3-dione (CZ47-02). The title compound (yield 90%) was synthesized using the same procedure for the preparation of CZ47-01 as white solid. NMR (600 MHz, CD₃OD) δ 8.34 (d, J=7.8 Hz, 1H), 8.18 (s, 1H), 7.96 (d, J=7.8 Hz, 1H), 7.69 (t, J=7.8 Hz, 1H), 7.58 (t, J=7.8 Hz, 1H), 7.48-7.46 (m, 2H), 7.13 (d, J=7.8 Hz, 1H), 7.06 (d, J=7.2, 1H), 6.82 (s, 1H), 5.01 (dd, J=12.6, 4.8 Hz, 1H), 4.72 (d, J=12.6 Hz, 1H), 4.61-4.57 (m, 1H), 4.07 (d, J=12.6 Hz, 1H), 3.69 (brs, 2H), 3.38-3.34 (m, 1H), 3.19 (t, J=12.6 Hz, 1H), 2.99 (t, J=12.0 Hz, 1H), 2.86-2.62 (m, 6H), 2.17 (s, 3H), 2.01-1.99 (m, 1H), 1.76 (d, J=12.6 Hz, 2H), 1.61-1.52 (m, 2H), 1.28-1.24 (m, 12H). HRMS calcd for C₄₄H₅₀ClN₈O₈S [M+H⁺] 885.3155, found 885.3152.

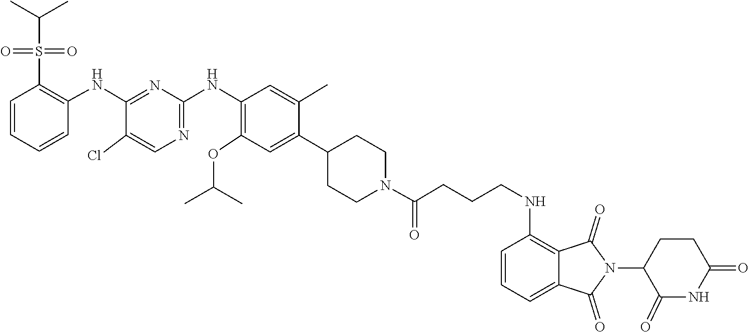 [SMILES: Cc1cc(Nc2ncc(Cl)c(Nc3ccccc3S(=O)(=O)C(C)C)n2)c(OC(C)C)cc1C1CCN(C(=O)CCCNc2cccc3c2C(=O)N(C2CCC(=O)NC2=O)C3=O)CC1]

4-((4-(4-(4-((5-Chloro-4-((2-(isopropylsulfonyl)phenyl)amino)pyrimidin-2-yl)amino)-5-isopropoxy-2-methylphenyl)piperidin-1-yl)-4-oxobutyl)amino)-2-(2,6-dioxopiperidin-3-yl)isoindoline-1,3-dione (CZ47-03). The title compound (yield 51%) was synthesized using the same procedure for the preparation of CZ47-01 as yellow solid. ¹H NMR (600 MHz, CD₃OD) δ 8.35 (d, J=7.8 Hz, 1H), 8.18 (s, 1H), 7.96 (d, J=7.8 Hz, 1H), 7.70 (t, J=7.8 Hz, 1H), 7.56 (t, J=7.8 Hz, 1H), 7.48 (s, 1H), 7.47 (t, J=7.8 Hz, 1H), 7.13 (d, J=7.8 Hz, 1H), 7.04 (d, J=7.2, 1H), 6.84 (s, 1H), 5.04 (dd, J=12.6, 4.8 Hz, 1H), 4.70 (d, J=12.6 Hz, 1H), 4.61-4.57 (m, 1H), 4.09 (d, J=12.6 Hz, 1H), 3.43 (brs, 2H), 3.38-3.34 (m, 1H), 3.20 (t, J=12.6 Hz, 1H), 3.00 (t, J=12.0 Hz, 1H), 2.85-2.78 (m, 1H), 2.74-2.65 (m, 3H), 2.61-2.51 (m, 2H), 2.17 (s, 3H), 2.07-2.05 (m, 1H), 2.01-1.96 (m, 2H), 1.78 (d, J=12.6 Hz, 2H), 1.61-1.53 (m, 2H), 1.27-1.24 (m, 12H). HRMS calcd for C₄₅H₅₂ClN₈O₈S [M+H⁺] 899.3312, found 899.3321.

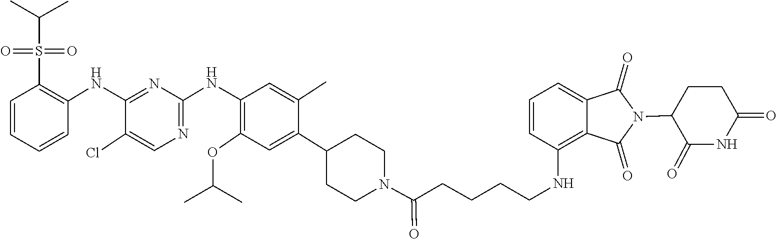 [SMILES: Cc1cc(Nc2ncc(Cl)c(Nc3ccccc3S(=O)(=O)C(C)C)n2)c(OC(C)C)cc1C1CCN(C(=O)CCCCNc2cccc3c2C(=O)N(C2CCC(=O)NC2=O)C3=O)CC1]

4-((5-(4-(4-((5-Chloro-4-((2-(isopropylsulfonyl)phenyl)amino)pyrimidin-2-yl)amino)-5-isopropoxy-2-methylphenyl)piperidin-1-yl)-5-oxopentyl)amino)-2-(2,6-dioxopiperidin-3-yl)isoindoline-1,3-dione (CZ47-04). The title compound (yield 60%) was synthesized using the same procedure for the preparation of CZ47-01 as yellow solid. ¹H NMR (600 MHz, CD₃OD) δ 8.39 (d, J=7.8 Hz, 1H), 8.16 (s, 1H), 7.94 (d, J=7.8 Hz, 1H), 7.69 (t, J=7.8 Hz, 1H), 7.62 (s, 1H), 7.55 (t, J=7.8 Hz, 1H), 7.42 (t, J=7.8 Hz, 1H), 7.07 (d, J=7.8 Hz, 1H), 7.02 (d, J=7.2 Hz, 1H), 6.82 (s, 1H), 5.00 (dd, J=12.6, 4.8 Hz, 1H), 4.69 (d, J=12.6 Hz, 1H), 4.61-4.57 (m, 1H), 4.05 (d, J=12.6 Hz, 1H), 3.43-3.34 (m, 3H), 3.20 (t, J=12.6 Hz, 1H), 2.99 (t, J=12.0 Hz, 1H), 2.82-2.76 (m, 1H), 2.72-2.61 (m, 3H), 2.54-2.47 (m, 2H), 2.16 (s, 3H), 2.04-2.00 (m, 1H), 1.80-1.75 (m, 6H), 1.61-1.53 (m, 2H), 1.29-1.24 (m, 12H). HRMS calcd for C₄₆H₅₄ClN₈O₈S [M+H⁺] 913.3468, found 913.3468.

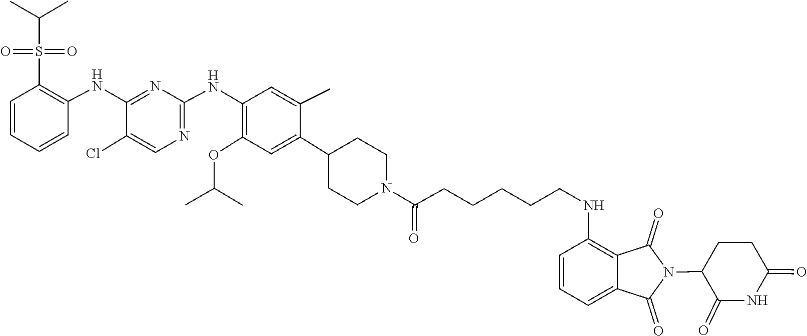 [SMILES: Cc1cc(Nc2ncc(Cl)c(Nc3ccccc3S(=O)(=O)C(C)C)n2)c(OC(C)C)cc1C1CCN(C(=O)CCCCCNc2cccc3c2C(=O)N(C2CCC(=O)NC2=O)C3=O)CC1]

4-((6-(4-(4-((5-Chloro-4-((2-(isopropylsulfonyl)phenyl)amino)pyrimidin-2-yl)amino)-5-isopropoxy-2-methylphenyl)piperidin-1-yl)-6-oxohexyl)amino)-2-(2,6-dioxopiperidin-3-yl)isoindoline-1,3-dione (CZ47-05). The title compound (yield 81%) was synthesized using the same procedure for the preparation of CZ47-01 as yellow solid. ¹H NMR (600 MHz, CD₃OD) δ 8.36 (d, J=7.8 Hz, 1H), 8.18 (s, 1H), 7.96 (d, J=7.8 Hz, 1H), 7.70 (t, J=7.8 Hz, 1H), 7.54 (t, J=7.8 Hz, 1H), 7.50 (s, 1H), 7.46 (t, J=7.8 Hz, 1H), 7.04 (d, J=7.8 Hz, 1H), 7.02 (d, J=7.2 Hz, 1H), 6.84 (s, 1H), 5.00 (dd, J=12.6, 4.8 Hz, 1H), 4.70 (d, J=12.6 Hz, 1H), 4.61-4.57 (m, 1H), 4.09 (d, J=12.6 Hz, 1H), 3.38-3.33 (m, 3H), 3.20 (t, J=12.6 Hz, 1H), 3.01 (t, J=12.0 Hz, 1H), 2.84-2.76 (m, 1H), 2.73-2.66 (m, 3H), 2.47 (t, J=7.2 Hz, 2H), 2.18 (s, 3H), 2.08-2.04 (m, 1H), 1.82-1.69 (m, 6H), 1.63-1.49 (m, 4H), 1.26-1.24 (m, 12H). HRMS calcd for C₄₇H₅₆ClN₈O₈S [M+H⁺] 927.3625, found 927.3640.

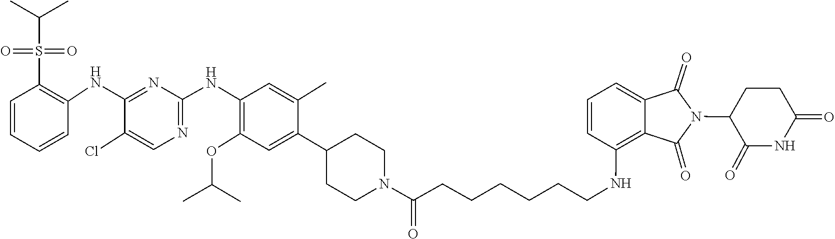 [SMILES: Cc1cc(Nc2ncc(Cl)c(Nc3ccccc3S(=O)(=O)C(C)C)n2)c(OC(C)C)cc1C1CCN(C(=O)CCCCCCNc2cccc3c2C(=O)N(C2CCC(=O)NC2=O)C3=O)CC1]

4-((7-(4-(4-((5-Chloro-4-((2-(isopropylsulfonyl)phenyl)amino)pyrimidin-2-yl)amino)-5-isopropoxy-2-methylphenyl)piperidin-1-yl)-7-oxoheptyl)amino)-2-(2,6-dioxopiperidin-3-yl)isoindoline-1,3-dione (CZ47-06). The title compound (yield 74%) was synthesized using the same procedure for the preparation of CZ47-01 as yellow solid. ¹H NMR (600 MHz, CD₃OD) δ 8.38 (d, J=7.8 Hz, 1H), 8.17 (s, 1H), 7.96 (d, J=7.8 Hz, 1H), 7.70 (t, J=7.8 Hz, 1H), 7.57 (s, 1H), 7.52 (t, J=7.8 Hz, 1H), 7.44 (t, J=7.8 Hz, 1H), 7.03 (d, J=8.4 Hz, 1H), 7.00 (d, J=7.2 Hz, 1H), 6.82 (s, 1H), 5.01 (dd, J=12.6, 4.8 Hz, 1H), 4.70 (d, J=12.6 Hz, 1H), 4.61-4.57 (m, 1H), 4.09 (d, J=12.6 Hz, 1H), 3.37-3.33 (m, 3H), 3.21 (t, J=12.6 Hz, 1H), 3.01 (t, J=12.0 Hz, 1H), 2.84-2.78 (m, 1H), 2.73-2.66 (m, 3H), 2.45 (t, J=7.2 Hz, 2H), 2.18 (s, 3H), 2.08-2.04 (m, 1H), 1.83-1.77 (m, 2H), 1.70-1.54 (m, 6H), 1.51-1.45 (m, 4H), 1.27-1.24 (m, 12H). HRMS calcd for C₄₈H₆₈ClN₈O₈S [M+H⁺] 941.3781, found 941.3779.

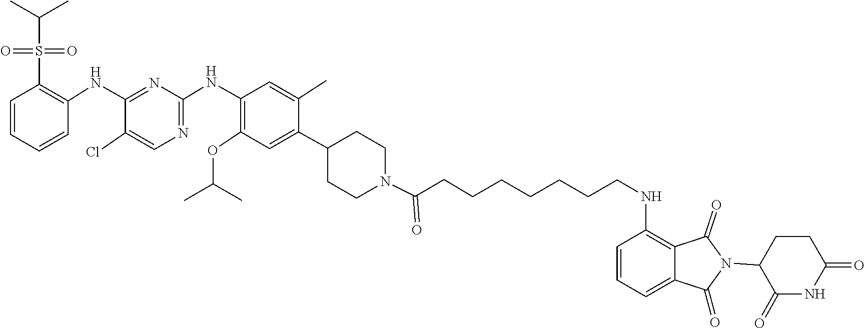 [SMILES: Cc1cc(Nc2ncc(Cl)c(Nc3ccccc3S(=O)(=O)C(C)C)n2)c(OC(C)C)cc1C1CCN(C(=O)CCCCCCCNc2cccc3c2C(=O)N(C2CCC(=O)NC2=O)C3=O)CC1]

4-((8-(4-(4-((5-Chloro-4-((2-(isopropylsulfonyl)phenyl)amino)pyrimidin-2-yl)amino)-5-isopropoxy-2-methylphenyl)piperidin-1-yl)-8-oxooctyl)amino)-2-(2,6-dioxopiperidin-3-yl)isoindoline-1,3-dione (CZ47-07). The title compound (yield 89%) was synthesized using the same procedure for the preparation of CZ47-01 as yellow solid. ¹H NMR (600 MHz, CD₃OD) δ 8.38 (d, J=7.8 Hz, 1H), 8.17 (s, 1H), 7.95 (d, J=7.8 Hz, 1H), 7.69 (t, J=7.8 Hz, 1H), 7.56 (s, 1H), 7.52 (t, J=7.8 Hz, 1H), 7.44 (t, J=7.8 Hz, 1H), 7.02 (d, J=8.4 Hz, 1H), 7.01 (d, J=7.2 Hz, 1H), 6.82 (s, 1H), 5.02 (dd, J=12.6, 4.8 Hz, 1H), 4.70 (d, J=12.6 Hz, 1H), 4.61-4.57 (m, 1H), 4.09 (d, J=12.6 Hz, 1H), 3.36-3.30 (m, 3H), 3.21 (t, J=12.6 Hz, 1H), 3.01 (t, J=12.0 Hz, 1H), 2.84-2.78 (m, 1H), 2.73-2.66 (m, 3H), 2.44 (t, J=7.2 Hz, 2H), 2.18 (s, 3H), 2.08-2.05 (m, 1H), 1.83-1.77 (m, 2H), 1.70-1.53 (m, 6H), 1.47-1.42 (m, 6H), 1.27-1.24 (m, 12H). HRMS calcd for C₄₉H₆₀ClN₈O₈S [M+H⁺] 955.3938, found 955.3939.

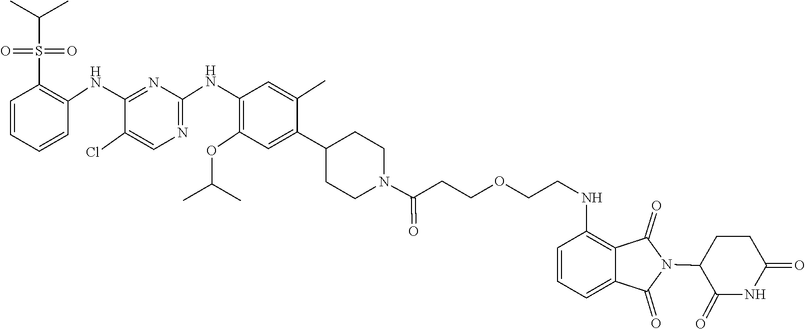 [SMILES: Cc1cc(Nc2ncc(Cl)c(Nc3ccccc3S(=O)(=O)C(C)C)n2)c(OC(C)C)cc1C1CCN(C(=O)CCOCCNc2cccc3c2C(=O)N(C2CCC(=O)NC2=O)C3=O)CC1]

4-((2-(3-(4-(4-((5-Chloro-4-((2-(isopropylsulfonyl)phenyl)amino)pyrimidin-2-yl)amino)-5-isopropoxy-2-methylphenyl)piperidin-1-yl)-3-oxopropoxy)ethyl)amino)-2-(2,6-dioxopiperidin-3-yl)isoindoline-1,3-dione (CZ47-08). The title compound (yield 86%) was synthesized using the same procedure for the preparation of CZ47-01 as yellow solid. ¹H NMR (600 MHz, CD₃OD) δ 8.36 (d, J=7.8 Hz, 1H), 8.18 (s, 1H), 7.96 (d, J=7.8 Hz, 1H), 7.71 (t, J=7.8 Hz, 1H), 7.53 (t, J=7.2 Hz, 1H), 7.52 (s, 1H), 7.47 (t, 7=7.8 Hz, 1H), 7.06 (t, J=7.8 Hz, 1H), 7.02 (t, J=7.2 Hz, 1H), 6.81 (s, 1H), 5.00 (dd, J=12.6, 4.8 Hz, 1H), 4.70 (d, J=12.6 Hz, 1H), 4.57-4.53 (m, 1H), 4.16 (d, J=12.6 Hz, 1H), 3.84-3.79 (m, 2H), 3.71 (brs, 2H), 3.51 (t, 2=5.4 Hz, 2H), 3.37-3.33 (m, 1H), 3.18 (t, J=12.6 Hz, 1H), 2.98 (t, J=12.0 Hz, 1H), 2.80-2.61 (m, 6H), 2.15 (s, 3H), 2.01-1.98 (m, 1H), 1.76 (d, J=12.6 Hz, 2H), 1.65-1.55 (m, 2H), 1.25-1.23 (m, 12H). HRMS calcd for C₄₆H₅₄ClN₈O₉S [M+H⁺] 929.3418, found 929.3426.

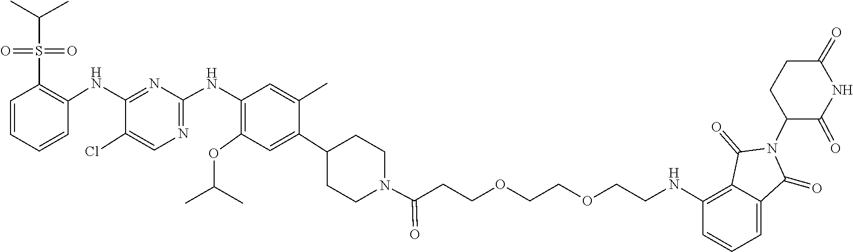 [SMILES: Cc1cc(Nc2ncc(Cl)c(Nc3ccccc3S(=O)(=O)C(C)C)n2)c(OC(C)C)cc1C1CCN(C(=O)CCOCCOCCNc2cccc3c2C(=O)N(C2CCC(=O)NC2=O)C3=O)CC1]

4-((2-(2-(3-(4-(4-((5-Chloro-4-((2-(isopropylsulfonyl)phenyl)amino)pyrimidin-2-yl)amino)-5-isopropoxy-2-methylphenyl)piperidin-1-yl)-3-oxopropoxy)ethoxy)ethyl)amino)-2-(2,6-dioxopiperidin-3-yl)isoindoline-1,3-dione (CZ47-09). The title compound (yield 82%) was synthesized using the same procedure for the preparation of CZ47-01 as yellow solid. ¹H NMR (600 MHz, CD₃OD) δ 8.32 (d, J=7.8 Hz, 1H), 8.19 (s, 1H), 7.97 (d, J=7.8 Hz, 1H), 7.71 (t, J=7.8 Hz, 1H), 7.50-7.48 (m, 2H), 7.42 (s, 1H), 7.03 (d, J=7.8 Hz, 1H), 6.99 (d, J=7.2 Hz, 1H), 6.85 (s, 1H), 5.00 (dd, J=12.6, 4.8 Hz, 1H), 4.69 (d, J=12.6 Hz, 1H), 4.62-4.57 (m, 1H), 4.12 (d, J=12.6 Hz, 1H), 3.79-3.75 (m, 2H), 3.68 (brs, 2H), 3.65 (brs, 4H), 3.44 (brs, 2H), 3.37-3.33 (m, 1H), 3.17 (t, J=12.6 Hz, 1H), 2.98 (t, J=12.0 Hz, 1H), 2.84-2.59 (m, 6H), 2.15 (s, 3H), 2.07-2.05 (m, 1H), 1.76 (t, J=12.6 Hz, 2H), 1.68-1.55 (m, 2H), 1.27-1.24 (m, 12H). HRMS calcd for C₄₈H₅₈ClN₈O₁₀S [M+H⁺]973.3680, found 973.3680.

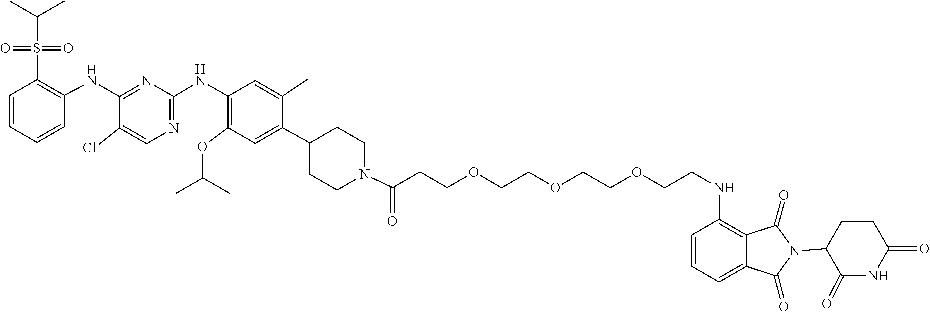 [SMILES: Cc1cc(Nc2ncc(Cl)c(Nc3ccccc3S(=O)(=O)C(C)C)n2)c(OC(C)C)cc1C1CCN(C(=O)CCOCCOCCOCCNc2cccc3c2C(=O)N(C2CCC(=O)NC2=O)C3=O)CC1]

4-((2-(2-(2-(3-(4-(4-((5-Chloro-4-((2-(isopropylsulfonyl)phenyl)amino)pyrimidin-2-yl)amino)-5-isopropoxy-2-methylphenyl)piperidin-1-yl)-3-oxopropoxy)ethoxy)ethoxy)ethyl)amino)-2-(2,6-dioxopiperidin-3-yl)isoindoline-1,3-dione (CZ47-10). The title compound (yield 79%) was synthesized using the same procedure for the preparation of CZ47-01 as yellow solid. ¹H NMR (600 MHz, CD₃OD) δ 8.33 (d, J=7.8 Hz, 1H), 8.18 (s, 1H), 7.97 (d, J=7.8 Hz, 1H), 7.71 (t, J=7.8 Hz, 1H), 7.51 (t, J=7.8 Hz, 1H), 7.48 (t, J=7.8 Hz, 1H), 7.44 (s, 1H), 7.04 (d, J=7.8 Hz, 1H), 7.01 (d, J=7.2 Hz, 1H), 6.86 (s, 1H), 5.02 (dd, J=12.6, 4.8 Hz, 1H), 4.69 (d, J=12.6 Hz, 1H), 4.62-4.57 (m, 1H), 4.13 (d, J=12.6 Hz, 1H), 3.79-3.75 (m, 2H), 3.68-3.66 (m, 2H), 3.63-3.61 (m, 8H), 3.44 (t, J=4.8 Hz, 2H), 3.37-3.33 (m, 1H), 3.19 (t, J=12.6 Hz, 1H), 3.00 (t, J=12.0 Hz, 1H), 2.84-2.59 (m, 6H), 2.17 (s, 3H), 2.08-2.06 (m, 1H), 1.76 (t, J=12.6 Hz, 2H), 1.69-1.56 (m, 2H), 1.27-1.24 (m, 12H). HRMS calcd for C₅₀H₆₂ClN₈O₁₁S [M+H⁺] 1017.3942, found 1017.3943.

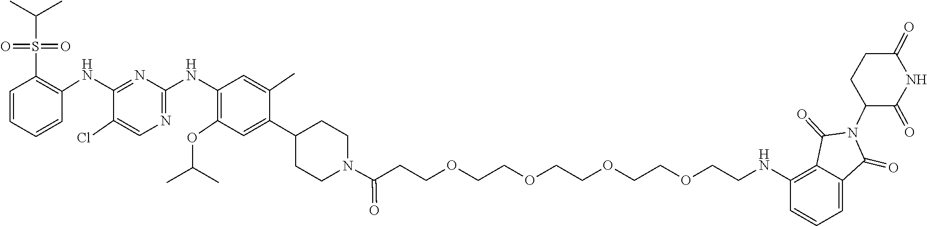 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCNc2cccc3c2C(=O)N(C2CCC(=O)NC2=O)C3=O)CC1]

4-((15-(4-(4-((5-Chloro-4-((2-(isopropylsulfonyl)phenyl)amino)pyrimidin-2-yl)amino)-5-isopropoxy-2-methylphenyl)piperidin-1-yl)-15-oxo-3,6,9,12-tetraoxapentadecyl)amino)-2-(2,6-dioxopiperidin-3-yl)isoindoline-1,3-dione (CZ47-11). The title compound (yield 80%) was synthesized using the same procedure for the preparation of CZ47-01 as yellow solid. ¹H NMR (600 MHz, CD₃OD) δ 8.36 (d, J=7.8 Hz, 1H), 8.17 (s, 1H), 7.95 (d, J=7.8 Hz, 1H), 7.69 (t, J=7.8 Hz, 1H), 7.53 (s, 1H), 7.50 (t, J=7.8 Hz, 1H), 7.45 (t, J=7.8 Hz, 1H), 7.05 (d, J=7.8 Hz, 1H), 7.01 (d, J=7.2 Hz, 1H), 6.84 (s, 1H), 5.02 (dd, J=12.6, 4.8 Hz, 1H), 4.70 (d, J=12.6 Hz, 1H), 4.62-4.57 (m, 1H), 4.14 (d, J=12.6 Hz, 1H), 3.78-3.73 (m, 2H), 3.68-3.66 (m, 2H), 3.61-3.59 (m, 12H), 3.45 (t, J=4.8 Hz, 2H), 3.37-3.33 (m, 1H), 3.20 (t, J=12.6 Hz, 1H), 3.00 (t, J=12.0 Hz, 1H), 2.86-2.61 (m, 6H), 2.16 (s, 3H), 2.08-2.06 (m, 1H), 1.78 (d, J=12.6 Hz, 2H), 1.69-1.54 (m, 2H), 1.28-1.24 (m, 12H). HRMS calcd for C₅₂H₆₆ClN₈O₁₂S [M+H⁺] 1061.4204, found 1061.4213.

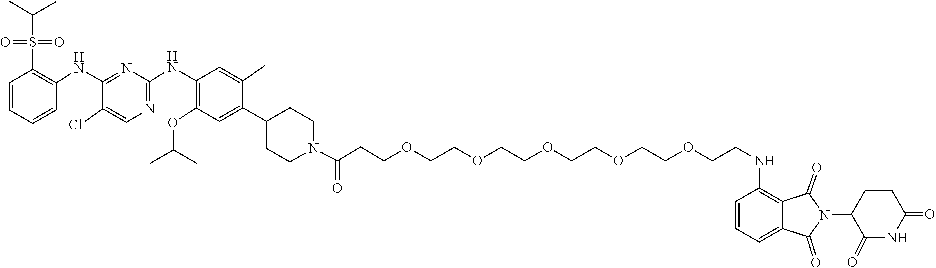 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCOCCNc2cccc3c2C(=O)N(C2CCC(=O)NC2=O)C3=O)CC1]

4-((18-(4-(4-((5-Chloro-4-((2-(isopropylsulfonyl)phenyl)amino)pyrimidin-2-yl)amino)-5-isopropoxy-2-methylphenyl)piperidin-1-yl)-18-oxo-3,6,9,12,15-pentaoxaoctadecyl)amino)-2-(2,6-dioxopiperidin-3-yl)isoindoline-1,3-dione (CZ47-12). The title compound (yield 72%) was synthesized using the same procedure for the preparation of CZ47-01 as yellow solid, ¹H NMR (600 MHz, CD₃OD) δ 8.36 (d, J=7.8 Hz, 1H), 8.17 (s, 1H), 7.95 (d, J=7.8 Hz, 1H), 7.69 (t, J=7.8 Hz, 1H), 7.53 (s, 1H), 7.50 (t, J=7.8 Hz, 1H), 7.44 (t, J=7.8 Hz, 1H), 7.05 (d, J=7.8 Hz, 1H), 7.01 (d, J=7.2 Hz, 1H), 6.84 (s, 1H), 5.02 (dd, J=12.6, 4.8 Hz, 1H), 4.70 (d, J=12.6 Hz, 1H), 4.62-4.57 (m, 1H), 4.15 (d, J=12.6 Hz, 1H), 3.78-3.73 (m, 2H), 3.69-3.67 (m, 2H), 3.62-3.57 (m, 16H), 3.45 (t, J=4.8 Hz, 2H), 3.37-3.33 (m, 1H), 3.21 (t, J=12.6 Hz, 1H), 3.00 (t, J=12.0 Hz, 1H), 2.83-2.62 (m, 6H), 2.17 (s, 3H), 2.08-2.06 (m, 1H), 1.78 (d, J=12.6 Hz, 2H), 1.69-1.54 (m, 2H), 1.28-1.24 (m, 12H). HRMS calcd for C₅₄H₇₀ClN₈O₁₃S [M+H⁺] 1105.4466, found 1105.4472.

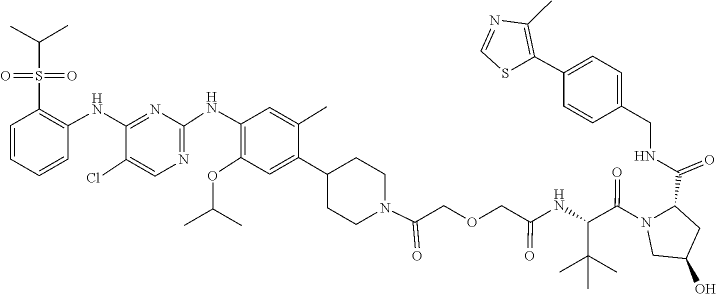 [SMILES: Cc1cc(Nc2ncc(Cl)c(Nc3ccccc3S(=O)(=O)C(C)C)n2)c(OC(C)C)cc1C1CCN(C(=O)COCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(2-(2-(4-(4-((5-Chloro-4-((2-(isopropylsulfonyl)phenyl)amino)pyrimidin-2-yl)amino)-5-isopropoxy-2-methylphenyl)piperidin-1-yl)-2-oxoethoxy)acetamido)-3,3-dimethylbutanoyl)-4-hydroxy-N-(4-(4-methylthiazol-5-yl)benzyl)pyrrolidine-2-carboxamide (CZ47-13). The title compound (yield 83%) was synthesized using the same procedure for the preparation of CZ47-01 as white solid. ¹H NMR (600 MHz, CD₃OD) δ 8.88 (d, J=8.4 Hz, 1H), 8.33 (s, 1H), 8.18 (s, 1H), 8.05 (t, J=8.4 Hz, 1H), 7.97 (d, J=8.4 Hz, 1H), 7.70 (t, J=7.2 Hz, 1H), 7.49-7.39 (m, 5H), 6.86 (s, 1H), 4.68 (d, J=13.2 Hz, 2H), 4.64-4.32 (m, 7H), 4.18-4.08 (m, 2H), 3.93-3.88 (m, 2H), 3.82-3.79 (m, 1H), 3.37-3.33 (m, 1H), 3.21 (t, J=12.6 Hz, 1H), 3.02 (t, J=12.0 Hz, 1H), 2.77 (t, J=12.6 Hz, 1H), 2.46 (s, 3H), 2.24-2.20 (m, 1H), 2.17 (s, 3H), 2.10-2.06 (m, 1H), 1.79 (t, J=14.4 Hz, 2H), 1.71-1.58 (m, 2H), 1.25-1.23 (m, 12H), 1.06 (s, 9H). HRMS calcd for C₅₄H₆₉ClN₉O₉S₂ [M+H⁺] 1086.4343, found 1086.4341.

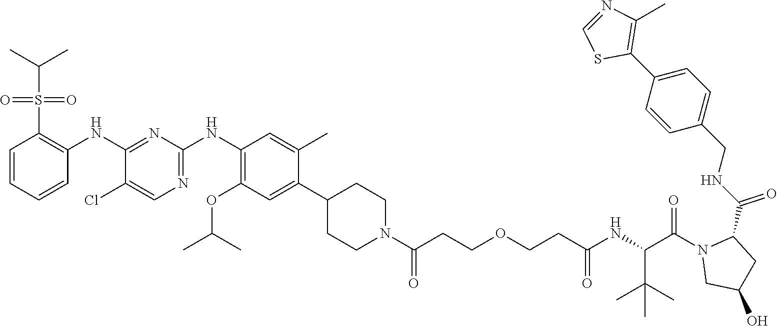 [SMILES: Cc1cc(Nc2ncc(Cl)c(Nc3ccccc3S(=O)(=O)C(C)C)n2)c(OC(C)C)cc1C1CCN(C(=O)CCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(3-(3-(4-(4-((5-Chloro-4-((2-(isopropylsulfonyl)phenyl)amino)pyrimidin-2-yl)amino)-5-isopropoxy-2-methylphenyl)piperidin-1-yl)-3-oxopropoxy)propanamido)-3,3-dimethylbutanoyl)-4-hydroxy-N-(4-(4-methylthiazol-5-yl)benzyl)pyrrolidine-2-carboxamide (CZ47-14). The title compound (yield 81%) was synthesized using the same procedure for the preparation of CZ47-01 as white solid. ¹H NMR (600 MHz, CD₃OD) δ 8.87 (s, 1H), 8.32 (s, 1H), 8.18 (s, 1H), 7.97 (d, J=7.8 Hz, 1H), 7.91 (d, J=8.4 Hz, 1H), 7.69 (t, J=8.4 Hz, 1H), 7.48-7.37 (m, 5H), 6.84 (s, 1H), 4.71 (d, J=12.6 Hz, 1H), 4.66 (d, J=12.6 Hz, 1H), 4.61-4.45 (m, 4H), 4.33 (d, J=15.6 Hz, 1H), 4.15 (d, J=12.6 Hz, 1H), 3.87 (d, J=10.8 Hz, 1H), 3.79-3.70 (m, 5H), 3.37-3.33 (m, 1H), 3.20 (t, J=12.6 Hz, 1H), 3.00 (t, J=12.0 Hz, 1H), 2.73-2.70 (m, 3H), 2.60-2.55 (m, 1H), 2.49-2.47 (m, 1H), 2.44 (s, 3H), 2.24-2.20 (m, 1H), 2.17 (s, 3H), 2.10-2.06 (m, 1H), 1.79 (t, J=14.4 Hz, 2H), 1.71-1.58 (m, 2H), 1.25-1.23 (m, 12H), 1.06 (s, 9H). HRMS calcd for C₅₆H₇₃ClN₉O₉S₂ [M+H⁺] 1114.4656, found 1114.4645.

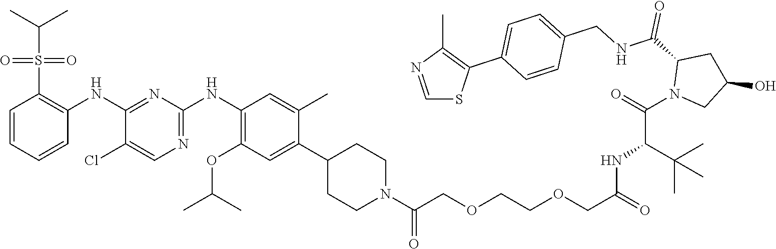 [SMILES: Cc1cc(Nc2ncc(Cl)c(Nc3ccccc3S(=O)(=O)C(C)C)n2)c(OC(C)C)cc1C1CCN(C(=O)COCCOCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(2-(2-(2-(4-(4-((5-Chloro-4-((2-(isopropylsulfonyl)phenyl)amino)pyrimidin-2-yl)amino)-5-isopropoxy-2-methylphenyl)piperidin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide (CZ47-15). The title compound (yield 80%) was synthesized using the same procedure for the preparation of CZ47-01 as white solid. ¹H NMR (600 MHz, CD₃OD) δ 8.86 (s, 1H), 8.30 (s, 1H), 8.17 (s, 1H), 7.95 (d, J=7.8 Hz, 1H), 7.74-7.68 (m, 2H), 7.51 (s, 1H), 7.46-7.37 (m, 4H), 6.84 (s, 1H), 4.71 (d, J=12.6 Hz, 1H), 4.64 (d, J=12.6 Hz, 1H), 4.60-4.48 (m, 3H), 4.44-4.30 (m, 4H), 4.11-4.01 (m, 3H), 3.85 (d, J=10.8 Hz, 1H), 3.79-3.70 (m, 5H), 3.37-3.33 (m, 1H), 3.18 (t, J=12.6 Hz, 1H), 3.00 (t, J=12.0 Hz, 1H), 2.75-2.70 (m, 1H), 2.44 (s, 3H), 2.24-2.20 (m, 1H), 2.16 (s, 3H), 2.10-2.06 (m, 1H), 1.79 (t, J=14.4 Hz, 2H), 1.71-1.58 (m, 2H), 1.25-1.23 (m, 12H), 1.06 (s, 9H). HRMS calcd for C₅₆H₇₃ClN₉O₁₀S₂ [M+H⁺] 1130.4605, found 1130.4617.

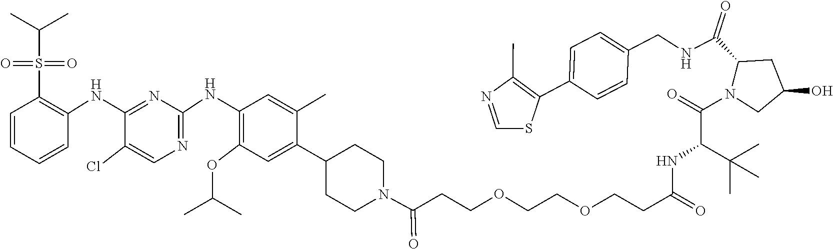 [SMILES: Cc1cc(Nc2ncc(Cl)c(Nc3ccccc3S(=O)(=O)C(C)C)n2)c(OC(C)C)cc1C1CCN(C(=O)CCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(3-(2-(3-(4-(4-((5-Chloro-4-((2-(isopropylsulfonyl)phenyl)amino)pyrimidin-2-yl)amino)-5-isopropoxy-2-methylphenyl)piperidin-1-yl)-3-oxopropoxy)ethoxy)propanamido)-3,3-dimethylbutanoyl)-4-hydroxy-N-(4-(4-methylthiazol-5-yl)benzyl)pyrrolidine-2-carboxamide (CZ47-16). The title compound (yield 86%) was synthesized using the same procedure for the preparation of CZ47-01 as white solid. ¹H NMR (600 MHz, CD₃OD) δ 8.87 (s, 1H), 8.37 (d, J=8.4 Hz, 1H),8.17 (s, 1H), 7.94 (d, J=7.8 Hz, 1H), 7.68 (t, J=7.8 Hz, 1H), 7.56 (s, 1H), 7.45-7.39 (m, 5H), 6.83 (s, 1H), 4.70 (d, J=12.6 Hz, 1H), 4.65 (d, J=12.6 Hz, 1H), 4.59-4.48 (m, 4H), 4.34 (d, J=15.6 Hz, 1H), 4.14 (d, J=12.6 Hz, 1H), 3.87 (d, J=10.8 Hz, 1H), 3.80-3.70 (m, 5H), 3.63-3.60 (m, 4H), 3.37-3.33 (m, 1H), 3.21 (t, J=12.6 Hz, 1H), 3.01 (t, J=12.0 Hz, 1H), 2.75-2.70 (m, 3H), 2.56-2.52 (m, 1H), 2.48-2.46 (m, 4H), 2.24-2.20 (m, 1H), 2.17 (s, 3H), 2.10-2.06 (m, 1H), 1.79 (t, J=14.4 Hz, 2H), 1.68-1.54 (m, 2H), 1.27-1.24 (m, 12H), 1.03 (s, 9H). HRMS calcd for C₅₈H₇₇ClN₉O₁₀S₂ [M+H⁺] 1158.4918, found 1158.4913.

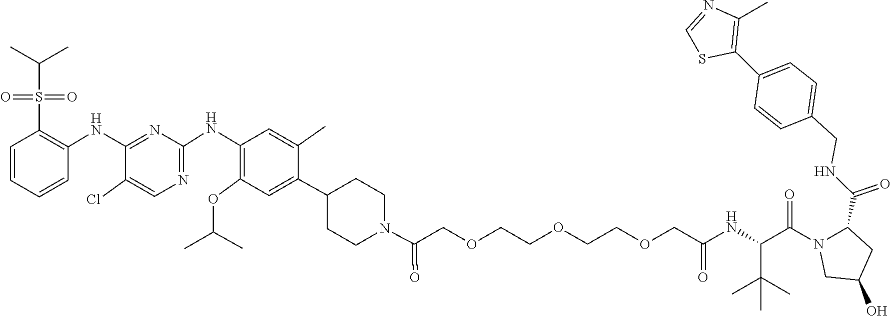 [SMILES: Cc1cc(Nc2ncc(Cl)c(Nc3ccccc3S(=O)(=O)C(C)C)n2)c(OC(C)C)cc1C1CCN(C(=O)COCCOCCOCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(tert-Butyl)-14-(4-(4-((5-chloro-4-((2-(isopropylsulfonyl)phenyl)amino)pyrimidin-2-yl)amino)-5-isopropoxy-2-methylphenyl)piperidin-1-yl)-4,14-dioxo-6,9,12-trioxa-3-azatetradecanoyl)-4-hydroxy-N-(4-(4-methylthiazol-5-yl)benzyl)pyrrolidine-2-carboxamide (CZ47-17). The title compound (yield 72%) was synthesized using the same procedure for the preparation of CZ47-01 as white solid. ¹H NMR (600 MHz, CD₃OD) δ 8.86 (s, 1H), 8.40 (d, J=7.8 Hz, 1H), 8.16 (s, 1H), 7.93 (d, J=7.8 Hz, 1H), 7.68-7.65 (m, 2H), 7.44-7.39 (m, 5H), 6.82 (s, 1H), 4.69 (d, J=12.6 Hz, 1H), 4.64 (d, J=12.6 Hz, 1H), 4.59-4.49 (m, 4H), 4.36-4.24 (m, 3H), 4.06-3.98 (m, 3H), 3.86 (d, J=10.8 Hz, 1H), 3.78 (dd, J=10.8, 4.8 Hz, 1H), 3.73-3.70 (m, 8H), 3.37-3.33 (m, 1H), 3.17 (t, J=12.6 Hz, 1H), 3.00 (t, J=12.0 Hz, 1H), 2.76-2.72 (m, 1H), 2.45 (s, 3H), 2.24-2.20 (m, 1H), 2.16 (s, 3H), 2.10-2.06 (m, 1H), 1.79 (d, J=12.0 Hz, 2H), 1.71-1.58 (m, 2H), 1.29-1.25 (m, 12H), 1.03 (s, 9H). HRMS calcd for C₅₈H₇₇ClN₉O₁₁S₂ [M+H⁺ ]1174.4867, found 117.4861.

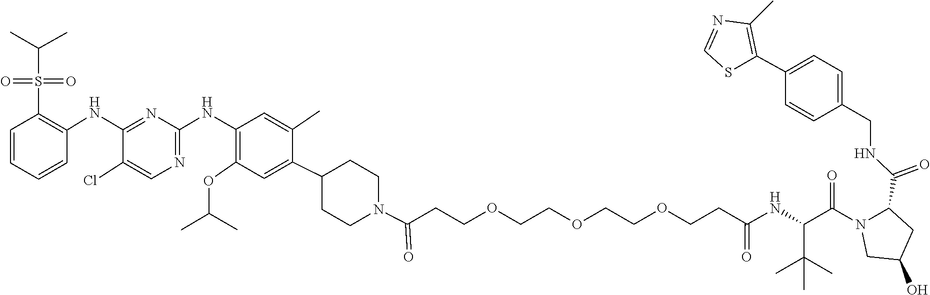 [SMILES: Cc1cc(Nc2ncc(Cl)c(Nc3ccccc3S(=O)(=O)C(C)C)n2)c(OC(C)C)cc1C1CCN(C(=O)CCOCCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(tert-Butyl)-16-(4-(4-((5-chloro-4-((2-(isopropylsulfonyl)phenyl)amino)pyrimidin-2-yl)amino)-5-isopropoxy-2-methylphenyl)piperidin-1-yl)-4,16-dioxo-7,10,13-trioxa-3-azahexadecanoyl)-4-hydroxy-N-(4-(4-methylthiazol-5-yl)benzyl)pyrrolidine-2-carboxamide (CZ47-18). The title compound (yield 83%) was synthesized using the same procedure for the preparation of CZ47-01 as white solid. ¹H NMR (600 MHz, CD₃OD) δ 8.87 (s, 1H), 8.39 (d, J=8.4 Hz, 1H), 8.16 (s, 1H), 7.93 (d, J=7.8 Hz, 1H), 7.68 (t, J=7.8 Hz, 1H), 7.63 (s, 1H), 7.45-7.39 (m, 5H), 6.83 (s, 1H), 4.71 (d, J=12.6 Hz, 1H), 4.64 (d, J=12.6 Hz, 1H), 4.59-4.48 (m, 4H), 4.34 (d, J=15.6 Hz, 1H), 4.15 (d, J=12.6 Hz, 1H), 3.87 (d, J=10.8 Hz, 1H), 3.80-3.66 (m, 5H), 3.63-3.58 (m, 8H), 3.36-3.32 (m, 1H), 3.21 (t, J=12.6 Hz, 1H), 3.00 (t, J=12.0 Hz, 1H), 2.76-2.65 (m, 3H), 2.57-2.53 (m, 1H), 2.48-2.46 (m, 4H), 2.24-2.20 (m, 1H), 2.17 (s, 3H), 2.09-2.05 (m, 1H), 1.79 (t, J=14.4 Hz, 2H), 1.68-1.54 (m, 2H), 1.29-1.24 (m, 12H), 1.02 (s, 9H). HRMS calcd for C₆₀H₈₁ClN₉O₁₁S₂ [M+H⁺] 1202.5180, found 1202.5173.

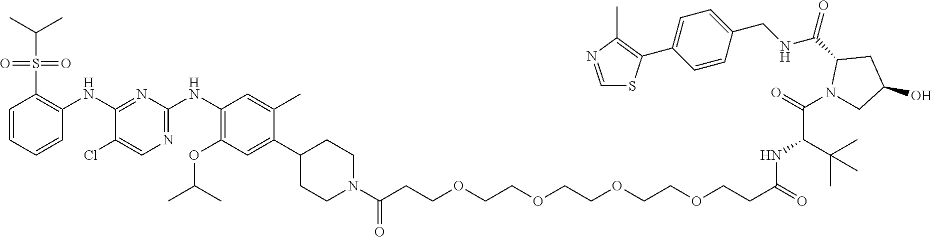 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(tert-Butyl)-19-(4-(4-((5-chloro-4-((2-(isopropylsulfonyl)phenyl)amino)pyrimidin-2-yl)amino)-5-isopropoxy-2-methylphenyl)piperidin-1-yl)-4,19-dioxo-7,10,13,16-tetraoxa-3-azanonadecanoyl)-4-hydroxy-N-(4-(4-methylthiazol-5-yl)benzyl)pyrrolidine-2-carboxamide (CZ47-19). The title compound (yield 80%) was synthesized using the same procedure for the preparation of CZ47-01 as white solid. ¹H NMR (600 MHz, CD₃OD) δ 8.86 (s, 1H), 8.42 (d, J=8.4 Hz, 1H), 8.15 (s, 1H), 7.92 (d, J=7.8 Hz, 1H), 7.71 (s, 1H), 7.66 (t, J=7.8 Hz, 1H), 7.45-7.36 (m, 5H), 6.81 (s, 1H), 4.71 (d, J=12.6 Hz, 1H), 4.64 (d, J=12.6 Hz, 1H), 4.60-4.48 (m, 4H), 4.34 (d, J=15.6 Hz, 1H), 4.15 (d, J=12.6 Hz, 1H), 3.87 (d, J=13.2 Hz, 1H), 3.79-3.68 (m, 5H), 3.61-3.58 (m, 12H), 3.36-3.32 (m, 1H), 3.21 (t, J=12.6 Hz, 1H), 3.00 (t, J=12.0 Hz, 1H), 2.78-2.64 (m, 3H), 2.57-2.53 (m, 1H), 2.45-2.43 (m, 4H), 2.22-2.18 (m, 1H), 2.16 (s, 3H), 2.09-2.05 (m, 1H), 1.79 (t, J=14.4 Hz, 2H), 1.68-1.54 (m, 2H), 1.30-1.24 (m, 12H), 1.02 (s, 9H). HRMS calcd for C₆₂H₈₅ClN₉O₁₂S₂ [M+H⁺] 1246.5442, found 1246.5446.

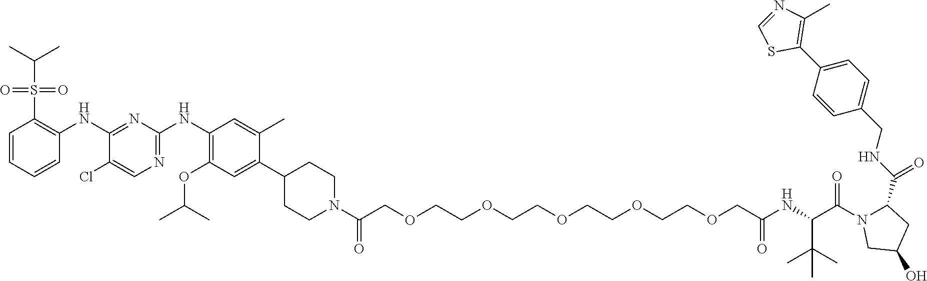 [SMILES: Cc1cc(Nc2ncc(Cl)c(Nc3ccccc3S(=O)(=O)C(C)C)n2)c(OC(C)C)cc1C1CCN(C(=O)COCCOCCOCCOCCOCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(tert-Butyl)-20-(4-(4-((5-chloro-4-((2-(isopropylsulfonyl)phenyl)amino)pyrimidin-2-yl)amino)-5-isopropoxy-2-methylphenyl)piperidin-1-yl)-4,20-dioxo-6,9,12,15,18-pentaoxa-3-azaicosanoyl)-4-hydroxy-N-(4-(4-methylthiazol-5-yl)benzyl)pyrrolidine-2-carboxamide (CZ47-20). The title compound (yield 83%) was synthesized using the same procedure for the preparation of CZ47-01 as white solid. ¹H NMR (600 MHz, CD₃OD) δ 8.86 (s, 1H), 8.42 (d, J=7.8 Hz, 1H), 8.15 (s, 1H), 7.92 (d, J=7.8 Hz, 1H), 7.70-7.64 (m, 2H), 7.45-7.37 (m, 5H), 6.82 (s, 1H), 4.69 (d, J=12.6 Hz, 1H), 4.64 (d, J=12.6 Hz, 1H), 4.59-4.49 (m, 4H), 4.36-4.32 (m, 2H), 4.24 (d, J=13.8 Hz, 1H), 4.06-3.98 (m, 3H), 3.86 (d, J=10.8 Hz, 1H), 3.78 (dd, J=10.8, 4.8 Hz, 1H), 3.68-3.60 (m, 16H), 3.37-3.33 (m, 1H), 3.18 (t, J=12.6 Hz, 1H), 3.00 (t, J=12.0 Hz, 1H), 2.76 (t, J=12.0 Hz, 1H), 2.46 (s, 3H), 2.24-2.20 (m, 1H), 2.16 (s, 3H), 2.10-2.06 (m, 1H), 1.79 (d, J=12.0 Hz, 2H), 1.71-1.58 (m, 2H), 1.30-1.24 (m, 12H), 1.03 (s, 9H). HRMS calcd for C₆₂H₈₅ClN₉O₁₃S₂ [M+H⁺] 1262.5391, found 1262.5398.

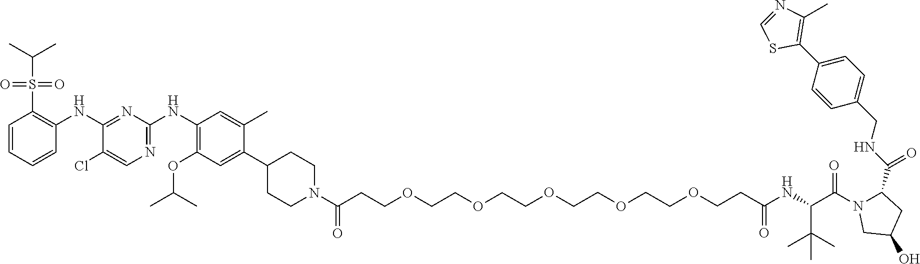 [SMILES: Cc1cc(Nc2ncc(Cl)c(Nc3ccccc3S(=O)(=O)C(C)C)n2)c(OC(C)C)cc1C1CCN(C(=O)CCOCCOCCOCCOCCOCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(tert-Butyl)-22-(4-(4-((5-chloro-4-((2-(isopropylsulfonyl)phenyl)amino)pyrimidin-2-yl)amino)-5-isopropoxy-2-methylphenyl)piperidin-1-yl)-4,22-dioxo-7,10,13,16,19-pentaoxa-3-azadocosanoyl)-4-hydroxy-N-(4-(4-methylthiazol-5-yl)benzyl)pyrrolidine-2-carboxamide (CZ47-21). The title compound (yield 85%) was synthesized using the same procedure for the preparation of CZ47-01 as white solid. ¹H NMR (600 MHz, CD₃OD) δ 8.86 (s, 1H), 8.42 (d, J=8.4 Hz, 1H), 8.15 (s, 1H), 7.92 (d, J=7.8 Hz, 1H), 7.70 (s, 1H), 7.67 (t, J=7.8 Hz, 1H), 7.45-7.36 (m, 5H), 6.81 (s, 1H), 4.71 (d, J=12.6 Hz, 1H), 4.64 (d, J=12.6 Hz, 1H), 4.60-4.48 (m, 4H), 4.34 (d, J=15.6 Hz, 1H), 4.15 (d, J=12.6 Hz, 1H), 3.87 (d, J=13.2 Hz, 1H), 3.79-3.67 (m, 5H), 3.61-3.58 (m, 16H), 3.36-3.32 (m, 1H), 3.22 (t, J=12.6 Hz, 1H), 3.00 (t, J=12.0 Hz, 1H), 2.78-2.64 (m, 3H), 2.57-2.53 (m, 1H), 2.45-2.43 (m, 4H), 2.22-2.18 (m, 1H), 2.16 (s, 3H), 2.09-2.05 (m, 1H), 1.79 (t, J=14.4 Hz, 2H), 1.68-1.54 (m, 2H), 1.30-1.24 (m, 12H), 1.02 (s, 9H). HRMS calcd for C₆₄H₈₉ClN₉O₁₃ S₂ [M+H⁺] 1290.5704, found 1290.5708.

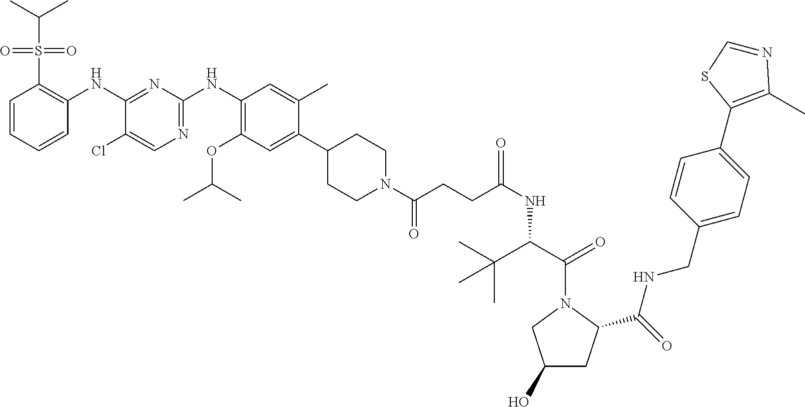 [SMILES: Cc1cc(Nc2ncc(Cl)c(Nc3ccccc3S(=O)(=O)C(C)C)n2)c(OC(C)C)cc1C1CCN(C(=O)CCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(4-(4-(4-((5-Chloro-4-((2-(isopropylsulfonyl)phenyl)amino)pyrimidin-2-yl)amino)-5-isopropoxy-2-methylphenyl)piperidin-1-yl)-4-oxobutanamido)-3,3-dimethylbutanoyl)-4-hydroxy-N-(4-(4-methylthiazol-5-yl)benzyl)pyrrolidine-2-carboxamide (CZ47-22). The title compound (yield 88%) was synthesized using the same procedure for the preparation of CZ47-01 as white solid. ¹H NMR (600 MHz, CD₃OD) δ 8.90 (s, 1H), 8.35 (d, J=7.8 Hz, 1H), 8.18 (s, 1H), 7.96 (d, J=7.8 Hz, 1H), 7.70 (t, J=7.8 Hz, 1H), 7.48-7.41 (m, 6H), 6.86 (d, J=7.8 Hz, 1H), 4.67 (d, J=12.6 Hz, 1H), 4.63-4.48 (m, 5H), 4.36 (d, J=15.6 Hz, 1H), 4.13 (d, J=13.2 Hz, 1H), 3.88 (d, J=10.8 Hz, 1H), 3.78 (dd, J=10.8, 4.8 Hz, 1H), 3.37-3.34 (m, 1H), 3.22 (t, J=12.6 Hz, 1H), 3.02 (t, J=12.0 Hz, 1H), 2.80-2.54 (m, 5H), 2.47 (s, 3H), 2.23-2.16 (m, 4H), 2.10-2.06 (m, 1H), 1.82 (d, J=12.0 Hz, 1H), 1.77 (d, J=12.0 Hz, 1H), 1.71-1.54 (m, 2H), 1.27-1.24 (m, 12H), 1.04 (s, 9H). HRMS calcd for C₅₄H₆₉ClN₉O₈S₂ [M+H⁺] 1070.4394, found 1070.4388.

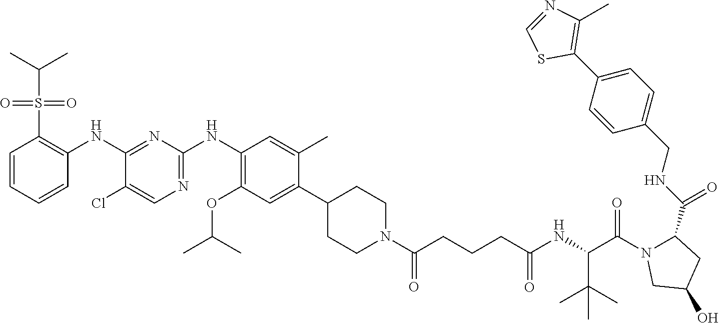 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(5-(4-(4-((5-Chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-4-hydroxy-N-(4-(4-methylthiazol-5-yl)benzyl)pyrrolidine-2-carboxamide (CZ47-23). The title compound (yield 83%) was synthesized using the same procedure for the preparation of CZ47-01 as white solid. ¹H NMR (600 MHz, CD₃OD) δ 8.91 (s, 1H), 8.33 (d, J=7.8 Hz, 1H), 8.19 (s, 1H), 7.97 (d, J=7.8 Hz, 1H), 7.70 (t, J=7.8 Hz, 1H), 7.49-7.38 (m, 6H), 6.86 (s, 1H), 4.70 (d, J=12.6 Hz, 1H), 4.62-4.49 (m, 5H), 4.34 (d, J=15.6 Hz, 1H), 4.10 (d, J=13.2 Hz, 1H), 3.91 (d, J=10.8 Hz, 1H), 3.80 (dd, J=10.8, 4.8 Hz, 1H), 3.37-3.34 (m, 1H), 3.22 (t, J=12.6 Hz, 1H), 3.02 (t, J=12.0 Hz, 1H), 2.72 (t, J=12.6 Hz, 1H), 2.48-2.32 (m, 7H), 2.22-2.14 (m, 4H), 2.11-2.05 (m, 1H), 1.94-1.90 (m, 2H), 1.82 (d, J=12.0 Hz, 1H), 1.78 (d, J=12.0 Hz, 1H), 1.71-1.54 (m, 2H), 1.27-1.24 (m, 12H), 1.04 (s, 9H). HRMS calcd for C₅₅H₇₁ClN₉O₈S₂ [M+H⁺] 1084.4550, found 1084.4553.

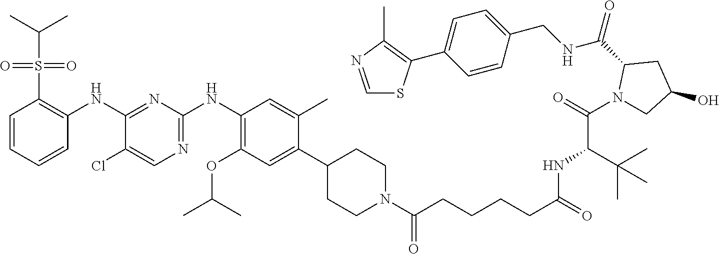 [SMILES: Cc1cc(Nc2ncc(Cl)c(Nc3ccccc3S(=O)(=O)C(C)C)n2)c(OC(C)C)cc1C1CCN(C(=O)CCCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(6-(4-(4-((5-Chloro-4-((2-(isopropylsulfonyl)phenyl)amino)pyrimidin-2-yl)amino)-5-isopropoxy-2-methylphenyl)piperidin-1-yl)-6-oxohexanamido)-3,3-dimethylbutanoyl)-4-hydroxy-N-(4-(4-methylthiazol-5-yl)benzyl)pyrrolidine-2-carboxamide (CZ47-24). The title compound (yield 86%) was synthesized using the same procedure for the preparation of CZ47-01 as white solid. ¹H NMR (600 MHz, CD₃OD) δ 8.91 (s, 1H), 8.35 (d, J=7.8 Hz, 1H), 8.18 (s, 1H), 7.97 (d, J=7.8 Hz, 1H), 7.70 (t, J=7.8 Hz, 1H), 7.49-7.38 (m, 6H), 6.86 (s, 1H), 4.70 (d, J=12.6 Hz, 1H), 4.64-4.49 (m, 5H), 4.34 (d, J=15.6 Hz, 1H), 4.10 (d, J=13.2 Hz, 1H), 3.88 (d, J=10.8 Hz, 1H), 3.79 (dd, J=10.8, 4.8 Hz, 1H), 3.37-3.34 (m, 1H), 3.22 (t, J=12.6 Hz, 1H), 3.02 (t, J=12.0 Hz, 1H), 2.72 (t, J=12.6 Hz, 1H), 2.48-2.46 (m, 5H), 2.36-2.28 (m, 2H), 2.22-2.16 (m, 4H), 2.11-2.05 (m, 1H), 1.84 (d, J=12.0 Hz, 1H), 1.78 (d, J=12.0 Hz, 1H), 1.68-1.55 (m, 6H), 1.27-1.24 (m, 12H), 1.03 (s, 9H). HRMS calcd for C₅₆H₇₃ClN₉O₈S₂ [M+H⁺] 1098.4707, found 1098.4718.

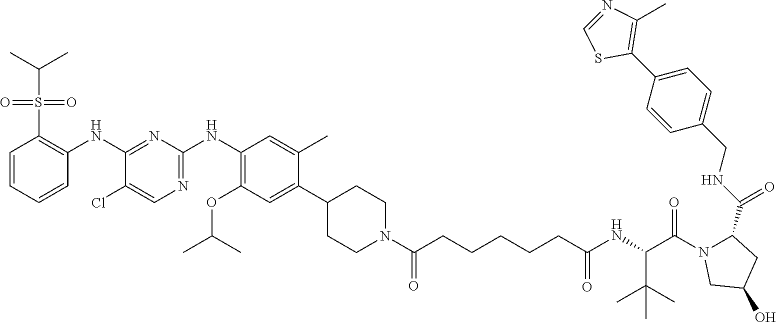 [SMILES: Cc1cc(Nc2ncc(Cl)c(Nc3ccccc3S(=O)(=O)C(C)C)n2)c(OC(C)C)cc1C1CCN(C(=O)CCCCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(7-(4-(4-((5-Chloro-4-((2-(isopropylsulfonyl)phenyl)amino)pyrimidin-2-yl)amino)-5-isopropoxy-2-methylphenyl)piperidin-1-yl)-7-oxoheptanamido)-3,3-dimethylbutanoyl)-4-hydroxy-N-(4-(4-methylthiazol-5-yl)benzyl)pyrrolidine-2-carboxamide (CZ47-25). The title compound (yield 89%) was synthesized using the same procedure for the preparation of CZ47-01 as white solid. ¹H NMR (600 MHz, CD₃OD) δ 8.91 (s, 1H), 8.33 (d, J=7.8 Hz, 1H), 8.18 (s, 1H), 7.97 (d, J=7.8 Hz, 1H), 7.70 (t, J=7.8 Hz, 1H), 7.49-7.38 (m, 6H), 6.86 (s, 1H), 4.70 (d, J=12.6 Hz, 1H), 4.64-4.49 (m, 5H), 4.35 (d, J=15.6 Hz, 1H), 4.10 (d, J=13.2 Hz, 1H), 3.89 (d, J=10.8 Hz, 1H), 3.79 (d, J=10.8 Hz, 1H), 3.37-3.34 (m, 1H), 3.23 (t, J=12.6 Hz, 1H), 3.02 (t, J=12.0 Hz, 1H), 2.72 (t, J=12.6 Hz, 1H), 2.48-2.45 (m, 5H), 2.34-2.26 (m, 2H), 2.22-2.16 (m, 4H), 2.11-2.05 (m, 1H), 1.83 (d, J=12.0 Hz, 1H), 1.78 (d, J=12.0 Hz, 1H), 1.68-1.55 (m, 6H), 1.43-1.38 (m, 2H), 1.27-1.24 (m, 12H), 1.03 (s, 9H). HRMS calcd for C₅₇H₇₅ClN₉O₈S₂ [M+H⁺] 1112.4863, found 1112.4864.

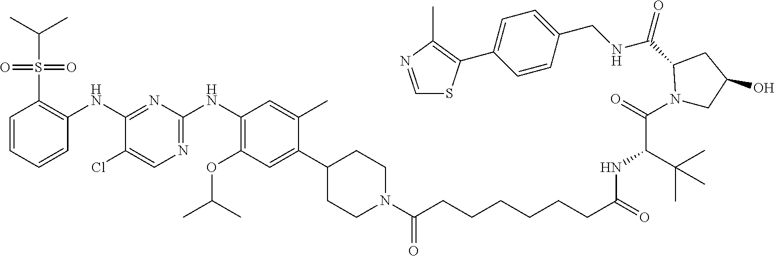 [SMILES: Cc1cc(Nc2ncc(Cl)c(Nc3ccccc3S(=O)(=O)C(C)C)n2)c(OC(C)C)cc1C1CCN(C(=O)CCCCCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(8-(4-(4-((5-Chloro-4-((2-(isopropylsulfonyl)phenyl)amino)pyrimidin-2-yl)amino)-5-isopropoxy-2-methylphenyl)piperidin-1-yl)-8-oxooctanamido)-3,3-dimethylbutanoyl)-4-hydroxy-N-(4-(4-methylthiazol-5-yl)benzyl)pyrrolidine-2-carboxamide (CZ47-26). The title compound (yield 88%) was synthesized using the same procedure for the preparation of CZ47-01 as white solid. ¹H NMR (600 MHz, CD₃OD) δ 8.90 (s, 1H), 8.34 (d, J=7.8 Hz, 1H), 8.18 (s, 1H), 7.97 (d, J=7.8 Hz, 1H), 7.70 (t, J=7.8 Hz, 1H), 7.49-7.38 (m, 6H), 6.85 (s, 1H), 4.70 (d, J=12.6 Hz, 1H), 4.64-4.49 (m, 5H), 4.35 (d, J=15.6 Hz, 1H), 4.10 (d, J=13.2 Hz, 1H), 3.89 (d, J=10.8 Hz, 1H), 3.79 (dd, J=10.8, 4.8 Hz, 1H), 3.37-3.34 (m, 1H), 3.23 (t, J=12.6 Hz, 1H), 3.02 (t, J=12.0 Hz, 1H), 2.72 (t, J=12.6 Hz, 1H), 2.48-2.43 (m, 5H), 2.34-2.24 (m, 2H), 2.22-2.16 (m, 4H), 2.11-2.05 (m, 1H), 1.83 (d, J=12.0 Hz, 1H), 1.78 (d, J=12.0 Hz, 1H), 1.65-1.55 (m, 6H), 1.38-1.33 (m, 4H), 1.27-1.24 (m, 12H), 1.03 (s, 9H). HRMS calcd for C₅₈H₇₇ClN₉O₈S₂ [M+H⁺] 1126.5020, found 1126.5018.

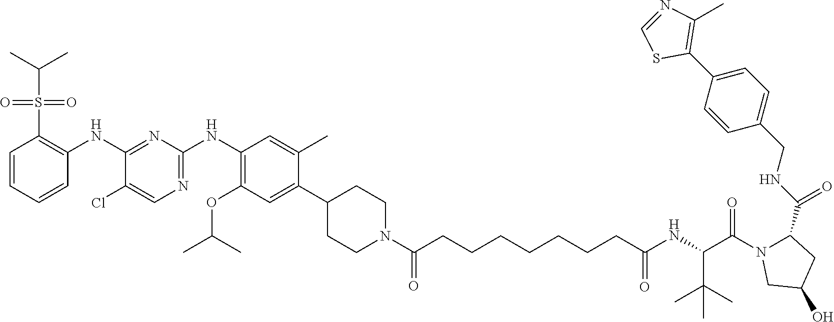 [SMILES: Cc1cc(Nc2ncc(Cl)c(Nc3ccccc3S(=O)(=O)C(C)C)n2)c(OC(C)C)cc1C1CCN(C(=O)CCCCCCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(9-(4-(4-((5-Chloro-4-((2-(isopropylsulfonyl)phenyl)amino)pyrimidin-2-yl)amino)-5-isopropoxy-2-methylphenyl)piperidin-1-yl)-9-oxononanamido)-3,3-dimethylbutanoyl)-4-hydroxy-7V-(4-(4-methylthiazol-5-yl)benzyl)pyrrolidine-2-carboxamide (CZ47-27). The title compound (yield 87%) was synthesized using the same procedure for the preparation of CZ47-01 as white solid. ¹H NMR (600 MHz, CD₃OD) δ 8.88 (s, 1H), 8.37 (d, J=7.8 Hz, 1H), 8.17 (s, 1H), 7.95 (d, J=7.8 Hz, 1H), 7.69 (t, J=7.8 Hz, 1H), 7.55 (s, 1H), 7.46-7.39 (m, 5H), 6.83 (s, 1H), 4.69 (d, J=12.6 Hz, 1H), 4.64-4.49 (m, 5H), 4.35 (d, J=15.6 Hz, 1H), 4.10 (d, J=13.2 Hz, 1H), 3.89 (d, J=10.8 Hz, 1H), 3.79 (dd, J=10.8, 4.8 Hz, 1H), 3.37-3.34 (m, 1H), 3.22 (t, J=12.6 Hz, 1H), 3.01 (t, J=12.0 Hz, 1H), 2.71 (t, J=12.6 Hz, 1H), 2.46-2.42 (m, 5H), 2.32-2.17 (m, 6H), 2.11-2.05 (m, 1H), 1.83 (d, J=12.0 Hz, 1H), 1.77 (d, J=12.0 Hz, 1H), 1.62-1.53 (m, 6H), 1.37 (brs, 6H), 1.28-1.24 (m, 12H), 1.03 (s, 9H). HRMS calcd for C₅₉H₇₉ClN₉O₈S₂ [M+H⁺] 1140.5176, found 1140.5182.

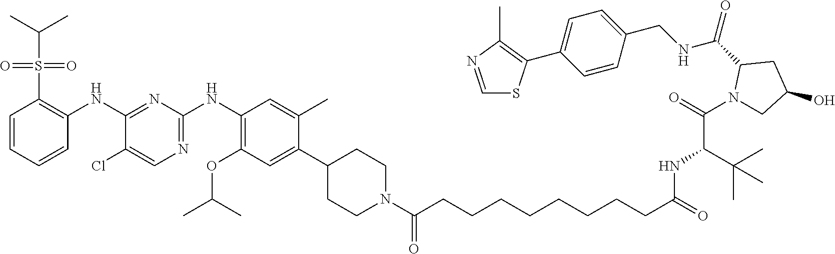

(2S,4R)-1-((S)-2-(10-(4-(4-((5-Chloro-4-((2-(isopropylsulfonyl)phenyl)amino)pyrimidin-2-yl)amino)-5-isopropoxy-2-methylphenyl)piperidin-1-yl)-10-oxodecanamido)-3,3-dimethylbutanoyl)-4-hydroxy-N-(4-(4-methylthiazol-5-yl)benzyl)pyrrolidine-2-carboxamide (CZ47-28). The title compound (yield 86%) was synthesized using the same procedure for the preparation of CZ47-01 as white solid. ¹H NMR (600 MHz, CD₃OD) δ 8.89 (s, 1H), 8.37 (d, J=7.8 Hz, 1H), 8.17 (s, 1H), 7.95 (d, J=7.8 Hz, 1H), 7.69 (t, J=7.8 Hz, 1H), 7.50 (s, 1H), 7.46-7.39 (m, 5H), 6.84 (s, 1H), 4.69 (d, J=12.6 Hz, 1H), 4.64-4.49 (m, 5H), 4.35 (d, J=15.6 Hz, 1H), 4.10 (d, J=13.2 Hz, 1H), 3.89 (d, J=10.8 Hz, 1H), 3.79 (dd, J=10.8, 4.8 Hz, 1H), 3.37-3.34 (m, 1H), 3.22 (t, J=12.6 Hz, 1H), 3.02 (t, J=12.0 Hz, 1H), 2.71 (t, J=12.6 Hz, 1H), 2.46-2.42 (m, 5H), 2.32-2.17 (m, 6H), 2.11-2.05 (m, 1H), 1.83 (d, J=12.0 Hz, 1H), 1.77 (d, J=12.0 Hz, 1H), 1.62-1.53 (m, 6H), 1.36 (brs, 8H), 1.28-1.24 (m, 12H), 1.03 (s, 9H). HRMS calcd for C₆₀H₈₁ClN₉O₈S₂ [M+H⁺] 1154.5333, found 1154.5340.

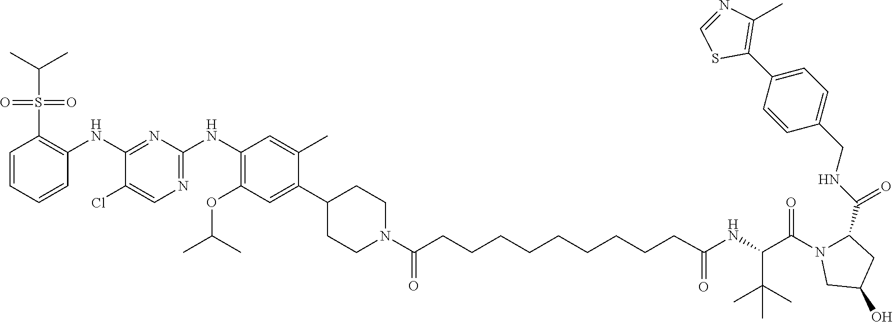 [SMILES: Cc1cc(Nc2ncc(Cl)c(Nc3ccccc3S(=O)(=O)C(C)C)n2)c(OC(C)C)cc1C1CCN(C(=O)CCCCCCCCCC(=O)N[C@H](C(=O)N2C[C@H](O)C[C@H]2C(=O)NCc2ccc(-c3scnc3C)cc2)C(C)(C)C)CC1]

(2S,4R)-1-((S)-2-(l l-(4-(4-((5-Chloro-4-((2-(isopropylsulfonyl)phenyl)amino)pyrimidin-2-yl)amino)-5-isopropoxy-2-methylphenyl)piperidin-1-yl)-11-oxoundecanamido)-3,3-dimethylbutanoyl)-4-hydroxy-N-(4-(4-methylthiazol-5-yl)benzyl)pyrrolidine-2-carboxamide (CZ47-29). The title compound (yield 64%) was synthesized using the same procedure for the preparation of CZ47-01 as white solid. ¹H NMR (600 MHz, CD₃OD) δ 8.90 (s, 1H), 8.35 (d, J=7.8 Hz, 1H), 8.18 (s, 1H), 7.96 (d, J=7.8 Hz, 1H), 7.70 (t, J=7.8 Hz, 1H), 7.47-7.40 (m, 6H), 6.84 (s, 1H), 4.70 (d, J=12.6 Hz, 1H), 4.63-4.48 (m, 5H), 4.35 (d, J=15.6 Hz, 1H), 4.10 (d, J=13.2 Hz, 1H), 3.89 (d, J=10.8 Hz, 1H), 3.79 (dd, J=10.8, 4.8 Hz, 1H), 3.37-3.34 (m, 1H), 3.23 (t, J=12.6 Hz, 1H), 3.01 (t, J=12.0 Hz, 1H), 2.72 (t, J=12.6 Hz, 1H), 2.46-2.42 (m, 5H), 2.30-2.18 (m, 6H), 2.11-2.05 (m, 1H), 1.83 (d, J=12.0 Hz, 1H), 1.78 (d, J=12.0 Hz, 1H), 1.64-1.54 (m, 6H), 1.36 (brs, 10H), 1.28-1.24 (m, 12H), 1.03 (s, 9H). HRMS calcd for C₆₁H₈₃ClN₉O₈S₂ [M+H⁺] 1168.5489, found 1168.5502.

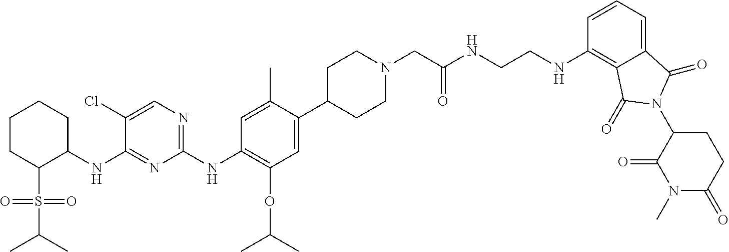 [SMILES: Cc1cc(Nc2ncc(Cl)c(NC3CCCCC3S(=O)(=O)C(C)C)n2)c(OC(C)C)cc1C1CCN(CC(=O)NCCNc2cccc3c2C(=O)N(C2CCC(=O)N(C)C2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(2-((2-(l-methyl-2,6-dioxopiperidin-3-yl)-1,3-dioxoisoindolin-4-yl)amino)ethyl)acetamide (CZ47-40). The title compound (yield 85%) was synthesized using the same procedure for the preparation of CZ40-50 as white solid. ¹H NMR (600 MHz, CD₃OD) δ 8.37 (d, J=8.4 Hz, 1H), 8.20 (s, 1H), 7.95 (d, J=7.8 Hz, 1H), 7.70 (t, J=1.2 Hz, 1H), 7.64 (s, 1H), 7.58 (t, J=7.8 Hz, 1H), 7.44 (t, J=7.8 Hz, 1H), 7.15 (d, J=9.0 Hz, 1H), 7.08 (d, J=6.6 Hz, 1H), 6.85 (s, 1H), 5.04 (dd, J=12.6, 5.4 Hz, 1H), 4.62-4.58 (m, 1H), 3.92 (s, 2H), 3.64 (d, J=10.2 Hz, 2H), 3.59-3.51 (m, 4H), 3.37-3.31 (m, 1H), 3.19 (t, J=12.0 Hz, 2H), 3.09-3.05 (m, 1H), 3.10 (s, 3H), 2.83-2.79 (m, 2H), 2.69-2.61 (m, 1H), 2.15 (s, 3H), 2.07-1.97 (m, 5H), 1.32 (d, J=6.0 Hz, 6H), 1.24 (d, J=7.2 Hz, 6H); ¹³C NMR (150 MHz, CD₃OD) δ 172.0, 169.9, 169.2, 167.7, 164.5, 160.8, 156.7, 154.9, 147.1, 146.6, 138.1, 137.0, 135.9, 134.7, 132.5, 131.2, 127.4, 126.9, 125.7, 125.1, 124.5, 124.0, 116.6, 111.0, 110.8, 110.0, 105.4, 71.4, 57.2, 55.4, 53.6, 49.4, 41.5, 38.2, 34.8, 31.0, 29.5, 25.7, 21.5, 20.9, 17.6, 13.9; HRMS calcd for C₄₆H₅₅ClN₉O₈S [M+H⁺] 928.3577, found 928.3584.

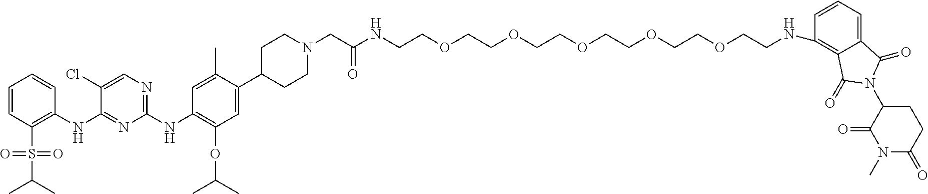 [SMILES: Cc1cc(Nc2ncc(Cl)c(Nc3ccccc3S(=O)(=O)C(C)C)n2)c(OC(C)C)cc1C1CCN(CC(=O)NCCOCCOCCOCCOCCOCCNc2cccc3c2C(=O)N(C2CCC(=O)N(C)C2=O)C3=O)CC1]

2-(4-(4-((5-Chloro-4-((2-(isopropylsulfonyl)phenyl)amino)pyrimidin-2-yl)amino)-5-isopropoxy-2-methylphenyl)piperidin-1-yl)-N-(17-((2-(l-methyl-2,6-dioxopiperidin-3-yl)-1,3-dioxoisoindolin-4-yl)amino)-3,6,9,12,15-pentaoxaheptadecyl)acetamide (MS4748). The title compound (yield 60%) was synthesized using the same procedure for the preparation of CZ40-50 as white solid. ¹H NMR (600 MHz, CD₃OD) δ 8.38 (d, J=8.4 Hz, 1H), 8.19 (s, 1H), 7.95 (d, J=7.8 Hz, 1H), 7.69 (t, J=7.8 Hz, 1H), 7.67 (s, 1H), 7.53 (t, J=7.8 Hz, 1H), 7.42 (t, J=7.8 Hz, 1H), 7.07 (d, J=8.4 Hz, 1H), 7.04 (d, J=7.2 Hz, 1H), 6.85 (s, 1H), 5.06 (dd, J=13.2, 5.4 Hz, 1H), 4.62-4.58 (m, 1H), 3.96 (s, 2H), 3.72-3.61 (m, 20H), 3.58 (t, J=5.4 Hz, 2H), 3.48-3.44 (m, 4H), 3.37-3.30 (m, 1H), 3.24 (t, J=11.4 Hz, 2H), 3.13 (s, 3H), 3.10-3.06 (m, 1H), 2.88-2.84 (m, 2H), 2.70-2.63 (m, 1H), 2.15 (s, 3H), 2.08-1.98 (m, 5H), 1.32 (d, J=6.0 Hz, 6H), 1.24 (d, J=7.2 Hz, 6H); ¹³C NMR (150 MHz, CD₃OD) δ 172.2, 170.0, 169.3, 167.8, 163.9, 161.0, 156.5, 155.5, 150.2, 146.7, 137.6, 137.2, 135.8, 134.7, 132.4, 131.2, 127.3, 126.5, 126.2, 124.7, 124.0, 123.1, 116.9, 110.8, 110.7, 109.8, 105.3, 71.4, 70.2, 70.1, 70.0, 69.8, 69.1, 68.7, 57.2, 55.4, 53.8, 49.4, 41.8, 39.1, 34.8, 31.1, 29.5, 26.0, 21.6, 20.9, 17.6, 14.0; HRMS calcd for C₅₆H₇₅ClN₉O₁₃S [M+H⁺] 1148.4888, found 1148.4880.

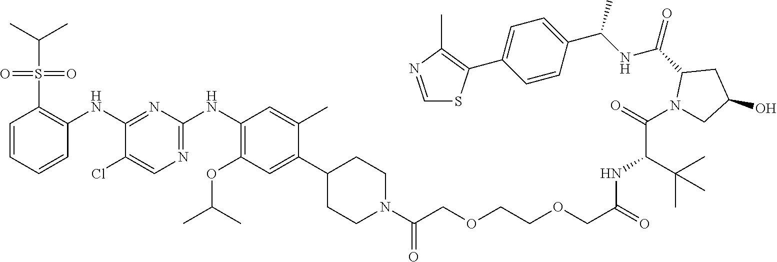

(2S,4R)-1-((S)-2-(2-(2-(2-(4-(4-((5-Chloro-4-((2-(isopropylsulfonyl)phenyl)amino)pyrimidin-2-yl)amino)-5-isopropoxy-2-methylphenyl)piperidin-1-yl)-2-oxoethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (HC58-98). The title compound (yield 72%) was synthesized using the same procedure for the preparation of CZ47-01 as white solid. ¹H NMR (600 MHz, CD₃OD) δ 8.99 (s, 1H), 8.31 (s, 1H), 8.22 (s, 1H), 8.00 (d, J=7.8 Hz, 1H), 7.79-7.66 (m, 2H), 7.53 (m, 1H), 7.47-7.35 (m, 4H), 6.91 (s, 1H), 4.75-4.55 (m, 4H), 4.50-4.34 (m, 3H), 4.17-4.03 (m, 3H), 3.90-3.72 (m, 6H), 3.43-35 (m, 1H), 3.26 (t, J=12.8 Hz, 1H), 3.13-3.01 (m, 1H), 2.86-2.77 (m, 1H), 2.49 (s, 3H), 2.27-2.18 (m, 5H), 2.01-1.93 (m, 1H), 1.91-1.78 (m, 2H), 1.79-1.68 (m, 1H), 1.68-1.56 (m, 1H), 1.46 (m, 3H), 1.34-1.23 (m, 12H), 1.06 (s, 9H). HRMS calcd for C₅₇H₇₅ClN₉O₁₀S₂ [M+H⁺] 1144.4767, found 1144.4769.

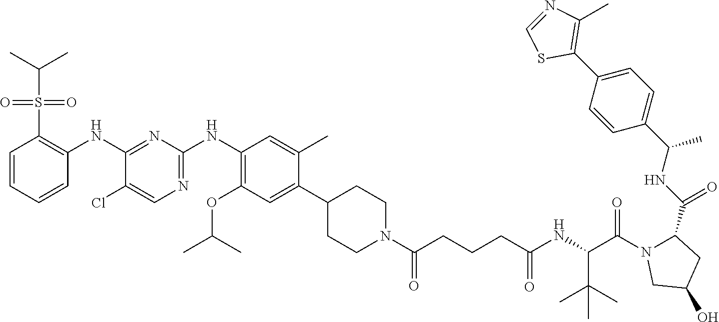 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@H](O)C[C@H]2C(=O)N[C@@H](C)c2ccc(-c3scnc3C)cc2)C(C)(C)C)CC1]

(2S,4R)-1-((S)-2-(5-(4-(4-((5-Chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (HC58-99). The title compound (yield 65%) was synthesized using the same procedure for the preparation of CZ47-01 as white solid. ¹H NMR (600 MHz, CD₃OD) δ 9.02 (s, 1H), 8.32 (s, 1H), 8.23 (s, 1H), 8.01 (d, J=8.1 Hz, 1H), 7.73 (s, 1H), 7.54 (t, J=1.1 Hz, 1H), 7.48-7.42 (m, 4H), 7.41-7.33 (m, 1H), 6.91 (s, 1H), 5.01 (m, 1H), 4.73 (d, J=13.1 Hz, 1H), 4.69-4.55 (m, 4H), 4.45 (s, 1H), 4.13 (d, J=14.3 Hz, 1H), 3.91 (d, J=11.0 Hz, 1H), 3.78 (d, J=11.0, 4.0 Hz, 1H), 3.43-3.37 (m, 1H), 3.25 (t, J=12.5 Hz, 1H), 3.06 (t, J=12.1 Hz, 1H), 2.76 (t, J=12.2 Hz, 1H), 2.50 (m, 4H), 2.45-2.33 (m, 2H), 2.27-2.18 (m, 4H), 2.02-1.91 (m, 3H), 1.90-1.79 (m, 2H), 1.75-1.64 (m, 1H), 1.59 (d, 7=7.1 Hz, 1H), 1.54-1.46 (m, 3H), 1.35-1.22 (m, 12H), 1.08 (s, 9H). HRMS calcd for C₅₆H₇₃ClN₉O₈S₂ [M+H⁺] 1098.4712, found 1098.4723.

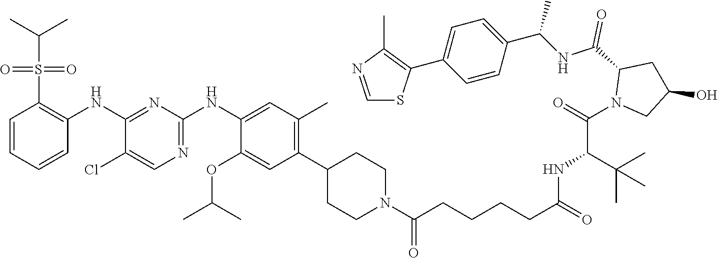 [SMILES: Cc1cc(Nc2ncc(Cl)c(Nc3ccccc3S(=O)(=O)C(C)C)n2)c(OC(C)C)cc1C1CCN(C(=O)CCCCC(=O)N[C@H](C(=O)N2C[C@H](O)C[C@H]2C(=O)N[C@@H](C)c2ccc(-c3scnc3C)cc2)C(C)(C)C)CC1]

(2S,4R)-1-(S)-2-(6-(4-(4-((5-Chloro-4-((2-(isopropylsulfonyl)phenyl)amino)pyrimidin-2-yl)amino)-5-isopropoxy-2-methylphenyl)piperidin-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (HC58-100). The title compound (yield 73%) was synthesized using the same procedure for the preparation of CZ47-01 as white solid. ¹H NMR (600 MHz, Methanol-d₄) δ 9.10 (s, 1H), 8.31 (s, 1H), 8.23 (s, 1H), 8.02 (d, J=8.0 Hz, 1H), 7.73 (s, 1H), 7.56 (t, J=7.7 Hz, 1H), 7.50-7.43 (m, 4H), 7.32 (s, 1H), 6.92 (s, 1H), 5.05-5.00 (m, 1H), 4.74 (d, J=13.0 Hz, 1H), 4.69-4.63 (m, 2H), 4.62-4.57 (m, 1H), 4.45 (s, 1H), 4.14 (d, J=13.4 Hz, 1H), 3.92-3.87 (m, 1H), 3.79-3.74 (m, 1H), 3.45-3.38 (m, 1H), 3.26 (t, J=12.8 Hz, 1H), 3.12-3.03 (m, 1H), 2.76 (t, J=12.8 Hz, 1H), 2.55-2.45 (m, 5H), 2.42-2.29 (m, 2H), 2.21 (d, J=19.8 Hz, 4H), 2.00-1.94 (m, 1H), 1.84 (m, 2H), 1.69 (s, 5H), 1.59 (d, J=7.3 Hz, 1H), 1.52 (d, J=7.1 Hz, 3H), 1.33-1.23 (m, 12H), 1.07 (s, 9H). HRMS calcd for C₅₇H₇₅ClN₉O₈S₂ [M+H⁺] 1112.4869, found 1112.4883.

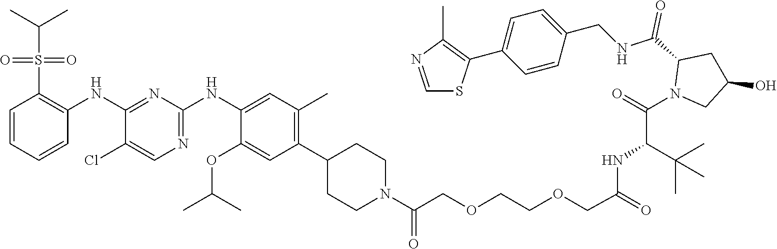 [SMILES: Cc1cc(Nc2ncc(Cl)c(Nc3ccccc3S(=O)(=O)C(C)C)n2)c(OC(C)C)cc1C1CCN(C(=O)COCCOCC(=O)N[C@H](C(=O)N2C[C@H](O)C[C@H]2C(=O)NCc2ccc(-c3scnc3C)cc2)C(C)(C)C)CC1]

(2S,4S)-1-((S)-2-(2-(2-(2-(4-(4-((5-Chloro-4-((2-(isopropylsulfonyl)phenyl)amino)pyrimidin-2-yl)amino)-5-isopropoxy-2-methylphenyl)piperidin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide (HC58-110). The title compound (yield 67%) was synthesized using the same procedure for the preparation of CZ47-01 as white solid. ¹H NMR (600 MHz, CD₃OD) δ 9.02 (s, 1H), 8.31 (s, 1H), 8.23 (s, 1H), 8.00 (d, J=8.0 Hz, 1H), 7.72 (s, 1H), 7.54 (t, J=7.7 Hz, 1H), 7.50-7.35 (m, 5H), 6.90 (s, 1H), 4.73 (d, J=14.0 Hz, 1H), 4.68-4.62 (m, 1H), 4.58-4.48 (m, 3H), 4.44-4.35 (m, 2H), 4.13 (d, J=14.0 Hz, 1H), 4.06 (dd, J=10.7, 5.1 Hz, 1H), 3.72 (dd, J=10.4, 3.8 Hz, 1H), 3.44-3.37 (m, 1H), 3.28-3.21 (m, 1H), 3.10-3.01 (m, 1H), 2.80-2.70 (m, 1H), 2.57-2.31 (m, 9H), 2.30-2.14 (m, 4H), 2.05-1.88 (m, 3H), 1.87-1.76 (m, 2H), 1.75-1.52 (m, 2H), 1.39-1.18 (m, 12H), 1.05 (s, 9H). HRMS calcd for C₅₆H₇₃ClN₉O₈S₂ [M+H⁺] 1130.4610, found 1130.4626.

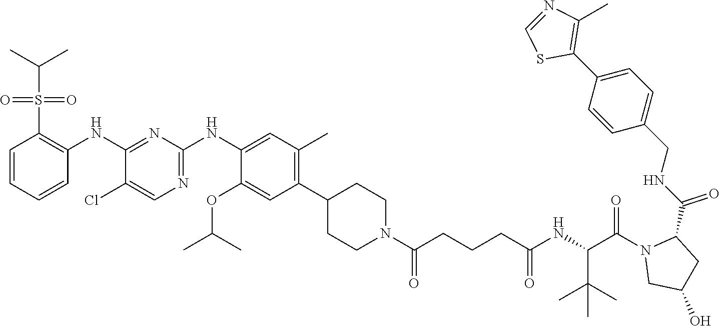 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@@H](O)C[C@H]2C(=O)NCc2ccc(-c3scnc3C)cc2)C(C)(C)C)CC1]

(2S,4S)-1-((S)-2-(5-(4-(4-((5-Chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-4-hydroxy-N-(4-(4-methylthiazol-5-yl)benzyl)pyrrolidine-2-carboxamide (HC58-111). The title compound (yield 78%) was synthesized using the same procedure for the preparation of CZ47-01 as white solid. ¹H NMR (600 MHz, CD₃OD) δ 8.97 (s, 1H), 8.29 (s, 1H), 8.23 (s, 1H), 8.00 (d, J=7.9 Hz, 1H), 7.75-7.68 (m, 2H), 7.54-7.50 (m, 1H), 7.42 (m, 4H), 6.88 (d, J=8.2 Hz, 1H), 4.70-4.58 (m, 3H), 4.55-4.30 (m, 4H), 4.13-3.96 (m, 3H), 3.83-3.70 (m, 6H), 3.42-3.34 (m, 1H), 3.27-3.18 (m, 1H), 3.10-2.97 (m, 1H), 2.83-2.72 (m, 1H), 2.53-2.38 (m, 4H), 2.20 (s, 3H), 1.99 (m, 1H), 1.81 (s, 2H), 1.74-1.53 (m, 2H), 1.33-1.18 (m, 12H), 1.06 (s, 9H). HRMS calcd for C₅₅H₇₁ClN₉O₈S₂ [M+H⁺] 1084.4556, found 1084.4547.

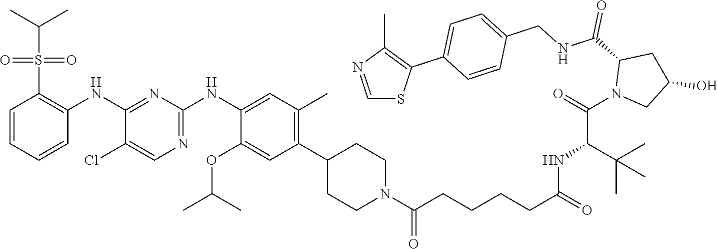 [SMILES: Cc1cc(Nc2ncc(Cl)c(Nc3ccccc3S(=O)(=O)C(C)C)n2)c(OC(C)C)cc1C1CCN(C(=O)CCCCC(=O)N[C@H](C(=O)N2C[C@@H](O)C[C@H]2C(=O)NCc2ccc(-c3scnc3C)cc2)C(C)(C)C)CC1]

(2S,4S)-1-((S)-2-(6-(4-(4-((5-chloro-4-((2-(isopropylsulfonyl)phenyl)amino)pyrimidin-2-yl)amino)-5-isopropoxy-2-methylphenyl)piperidin-1-yl)-6-oxohexanamido)-3,3-dimethylbutanoyl)-4-hydroxy-N-(4-(4-methylthiazol-5-yl)benzyl)pyrrolidine-2-carboxamide (HC58-112). The title compound (yield 81%) was synthesized using the same procedure for the preparation of CZ47-01 as white solid. ¹H NMR (600 MHz, CD₃OD) δ 8.99 (s, 1H), 8.33 (s, 1H), 8.23 (s, 1H), 8.02-7.99 (m, 1H), 7.72 (s, 1H), 7.53 (t, J=7.7 Hz, 1H), 7.47 (d, J=7.8 Hz, 2H), 7.43 (d, J=8.1 Hz, 2H), 7.38 (s, 1H), 6.90 (s, 1H), 4.73 (d, J=13.4 Hz, 1H), 4.70-4.62 (m, 1H), 4.59-4.50 (m, 2H), 4.44-4.33 (m, 2H), 4.13 (d, J=14.2 Hz, 1H), 4.07-3.98 (m, 1H), 3.71 (m, 1H), 3.45-3.36 (m, 1H), 3.31-3.19 (m, 1H), 3.12-2.99 (m, 1H), 2.75 (t, J=12.7 Hz, 1H), 2.56-2.28 (m, 7H), 2.22 (s, 3H), 2.07-1.95 (m, 1H), 1.90-1.76 (m, 2H), 1.76-1.55 (m, 8H), 1.37-1.19 (m, 12H), 1.06 (m, 9H). HRMS calcd for C₅₆H₇₃ClN₉O₈S₂ [M+H⁺] 1098.4712, found 1098.4717.

### Materials and Methods:

### General Chemistry Methods

HPLC spectra for all compounds were acquired using an Agilent 1200 Series system with DAD detector. Chromatography was performed on a 2.1×150 mm Zorbax 300SB-C18 5 μm column with water containing 0.1% formic acid as solvent A and acetonitrile containing 0.1% formic acid as solvent B at a flow rate of 0.4 ml/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Nuclear Magnetic Resonance (NMR) spectra were acquired on a Bruker DRX-600 spectrometer with 600 MHz for proton (¹H NMR) and 150 MHz for carbon (¹³C NMR); chemical shifts are reported in (δ). Preparative HPLC was performed on Agilent Prep 1200 series with UV detector set to 254 nm. Samples were injected onto a Phenomenex Luna 75×30 mm, 5 μm, C₁₈ column at room temperature. The flow rate was 40 ml/min. A linear gradient was used with 10% (or 50%) of MeOH (A) in H₂O (with 0.1% TFA) (B) to 100% of MeOH (A). HPLC was used to establish the purity of target compounds. All final compounds had >95% purity using the HPLC methods described above.

### Cell Culture

SU-DHL-1, NCI-H3122 and NCI-H2228 cells were cultured in RPMI-1640 medium supplemented with 10% fetal bovine serum, 50 μg/mL penicillin and streptomycin. Cells were cultured in a 37° C. incubator with 5% CO₂.

### Cell Viability Assay

SU-DHL-1 cells were seeded in 96-well plates at a density of 1×104 cells per well, in triplicate. Treated cells with DMSO or indicated serial dilutions of compounds for 2.5 days. Cell growth/survival was measured by using the CellTiter-Glo luminescent cell viability assay following the manufacturer's instructions (Promega) or CCK8 cell viability assay (Sigma). Data was analyzed by using the GraphPad Prism software, Error bars represent ±SD for triplicate experiments.

### Western Blot Assay

Cells were washed with cold PBS once and lysed in 1× Laemmli loading buffer directly. Lysates were heated at 99° C. for 5 mm, and resolved on 4-15% precast SDS-PAGE (BIO-RAD) and transferred onto PVDF membrane. Membrane was blocked in 5% milk in Tris-buffered saline and Tween 20 (TBST) for 1 h at room temperature, followed by incubation with a primary antibody overnight at 4° C., and a horseradish peroxidase (HRP)-conjugated secondary antibody for 1 h at room temperature. The membrane was imaged by a ChemiDoc MP imaging system (BIO-RAD). Antibodies against GAPDH (abeam), Phospho-ALK (Tyr1507), ALK (Cell Signaling Technology), Tubulin (Santa Cruz) were purchased commercially.

The ALK fusion protein degradation results of bivalent compounds are set forth in Table 2 below.

| Cpd | SU-DHL-1 | Cpd | NCI-H3122 | Cpd | NCI-H3122 |
|---|---|---|---|---|---|
| Code | (30 nM) | Code | (500 nM) | Code | (500 nM) |
| CZ40-50 | ++ | CZ47-01 | − | CZ47-22 | + |
| CZ40-51 | ++ | CZ47-02 | + | CZ47-23 | ++ |
| CZ40-52 | ++ | CZ47-03 | + | CZ47-24 | ++ |
| CZ40-53 | ++ | CZ47-04 | + | CZ47-25 | ++ |
| CZ40-77 | ++ | CZ47-05 | − | CZ47-26 | ++ |
| CZ40-78 | ++ | CZ47-06 | − | CZ47-27 | ++ |
| CZ40-79 | ++ | CZ47-07 | + | CZ47-28 | − |
| CZ40-80 | + | CZ47-08 | ++ | CZ47-29 | − |
| CZ40-81 | ++ | CZ47-09 | ++ | CZ47-40 | − |
| CZ40-82 | ++ | CZ47-10 | − | CZ47-48 | − |
| CZ40-83 | ++ | CZ47-11 | + | HC58-98 | ++ |
| CZ40-84 | + | CZ47-12 | + | HC58-99 | ++ |
| CZ40-85 | − | CZ47-13 | + | HC58-100 | ++ |
| CZ40-86 | − | CZ47-14 | + | HC58-110 | − |
| CZ40-87 | − | CZ47-15 | ++ | HC58-111 | − |
| CZ40-88 | + | CZ47-16 | − | HC58-112 | − |
| CZ40-89 | + | CZ47-17 | − | | |
| CZ40-90 | − | CZ47-18 | − | | |
| CZ40-92 | − | CZ47-19 | − | | |
| CZ40-93 | − | CZ47-20 | − | | |
| CZ40-94 | + | CZ47-21 | − | | |

Protein degradation >80%:++;
Protein degradation 20-80%:+;
Protein degradation <20%:−.

### Example 4. ALK Degraders Reduced NPM-ALK Fusion Protein Levels in SU-DHL-1 Cells at 30 nM and 100 nM (FIGS. 1 and 2)

SU-DHL-1 suspension cells were seeded in 12-well plates at a density of 6×10⁵/mL and treated with DMSO or indicated compounds at 30 nM or 100 nM for indicated time. Western blot results showed that various ALK degraders significantly reduced NPM-ALK fusion protein levels at 30 nM while the ALK activity inhibitor, ceritinib had no effect on ALK protein levels.

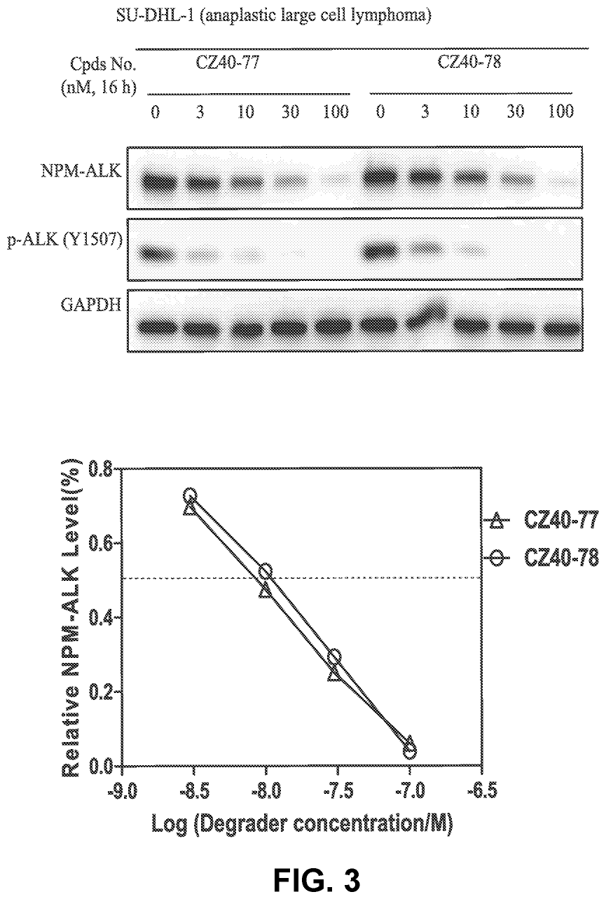

### Example 5. ALK Degraders Reduced NPM-ALK Fusion Protein Levels in SU-DHL-1 Cells in a Concentration-Dependent Manner (FIG. 3)

SU-DHL-1 cells were seeded in 12-well plates at a density of 4×10⁵/mL and treated with DMSO or indicated serial dilutions of compounds for 16 h. The NPM-ALK fusion protein levels were determined by Western blot and normalized against GPADH (right panel). Dose-responding curves showed that ALK degraders CZ40-77 and CZ40-78 resulted in rapid NPM-ALK fusion protein degradation with DC₅₀s (the drug concentration that results in 50% protein degradation) values less than 10 nM. Phosphorylation at Tyr1507 site is a measure of ALK activity, results showed that ALK degraders CZ40-77 and CZ40-78 also strongly inhibited the ALK activity at 3 nM.

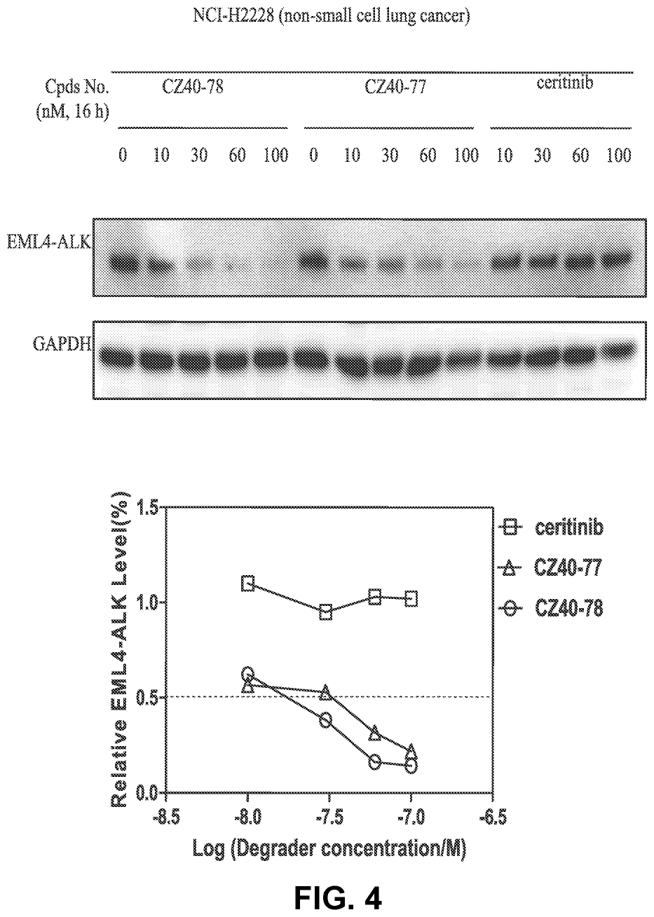

### Example 6. ALK Degraders Concentration-Dependently Reduced EML4-ALK v3 Fusion Protein Levels in NCI-H2228 Cells (FIG. 4)

NCI-H2228 cells were seeded in 12-well plates at a density of 2×10⁵/mL one day before treatment. Cells were 70% confluent and treated with DMSO or indicated serial dilutions of compounds for 16 h. The EML4-ALK fusion protein levels were determined by Western blot and normalized against GPADH (right panel). Dose-responding curves showed that ALK degraders CZ40-77 and CZ40-78 resulted in rapid EML4-ALK v3 fusion protein degradation with DC₅₀s values nearly 10 nM. Importantly, the ALK activity inhibitor, ceritinib showed no distinct effect on ALK protein levels at 100 nM.

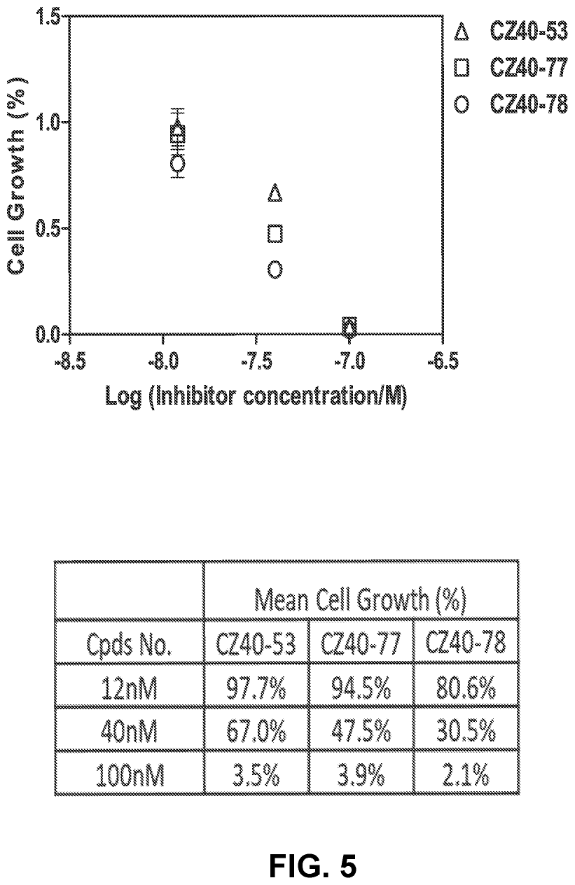

### Example 7. ALK Degraders Inhibited SU-DHL-1 Cell Growth (FIG. 5)

SU-DHL-1 cells were seeded in 96-well plates at a density of 1×10⁴ cells per well, in triplicate. Treated cells with DMSO or indicated serial dilutions of compounds for 2.5 days. Cell growth/survival was measured by using the CellTiter-Glo luminescent cell viability assay. Data was analyzed by using the GraphPad Prism software, Error bars represent ±SD for triplicate experiments (left panel). Dose-responding curves showed that ALK degraders CZ40-53, CZ40-77 and CZ40-78 significantly inhibited SU-DHL-1 cell growth with GI₅₀s (the drug concentration that results in 50% growth inhibition) values less than 40 nM.

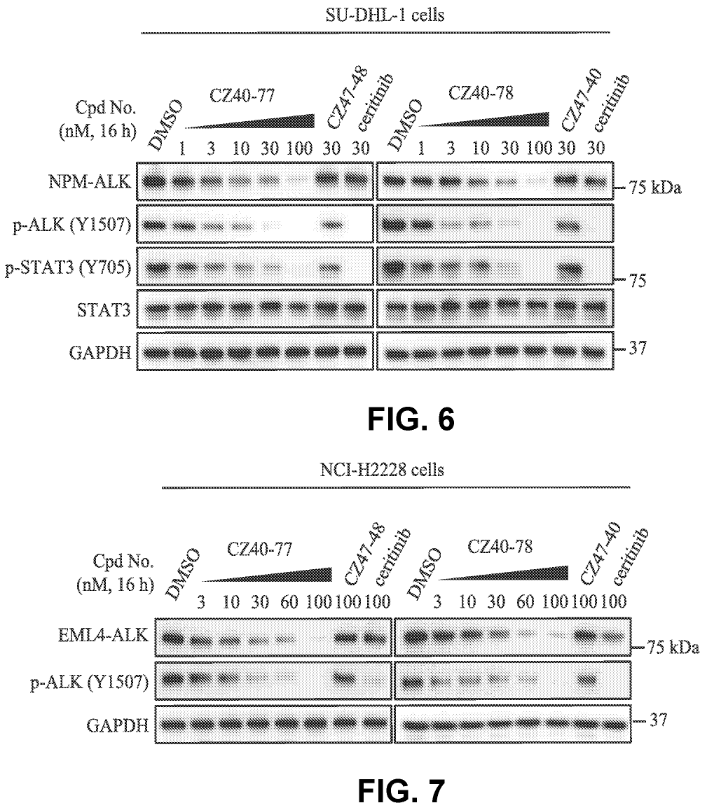

### Example 8. CZ40-77 and CZ40-78 Significantly Reduced ALK Fusion Protein Levels and Inhibited the ALK Down-Stream Signaling in a Concentration-Dependent Manner in SU-DHL-1 (FIG. 6) and NCI-H2228 (FIG. 7) Cells

SU-DHL-1 or NCI-H2228 cells were treated with DMSO or indicated compounds at 1, 3, 10, 30 nM, or 100 nM for 16 h. Western blot results showed that CZ40-77 and CZ40-78 concentration-dependently reduced NPM-ALK or EML4-ALK fusion protein levels, while the negative control compounds CZ47-40 and CZ47-48, as well as ALK activity inhibitor, ceritinib had no significant effect on ALK protein levels.

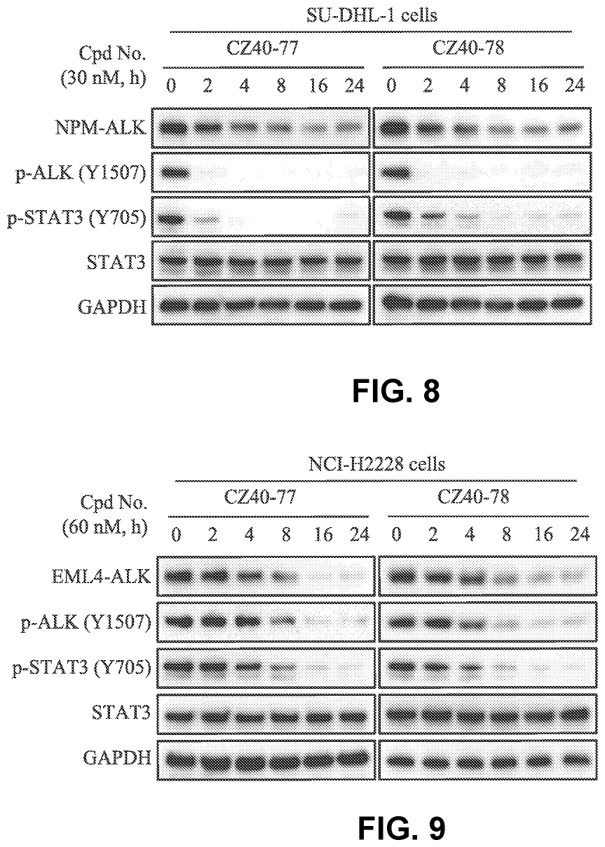

### Example 9. CZ40-77 and CZ40-78 Significantly Reduced ALK Fusion Protein Levels and Inhibited the ALK Down-Stream Signaling in a Time-Dependent Manner in SU-DHL-1 (FIG. 8) and NCI-H2228 (FIG. 9) Cells

SU-DHL-1 or NCI-H2228 cells were treated with CZ40-77 and CZ40-78 at indicated concentration for 0, 2, 4, 8, 16 and 24 h. Western blot results showed that CZ40-77 and CZ40-78 time-dependently reduced NPM-ALK or EML4-ALK fusion protein levels.

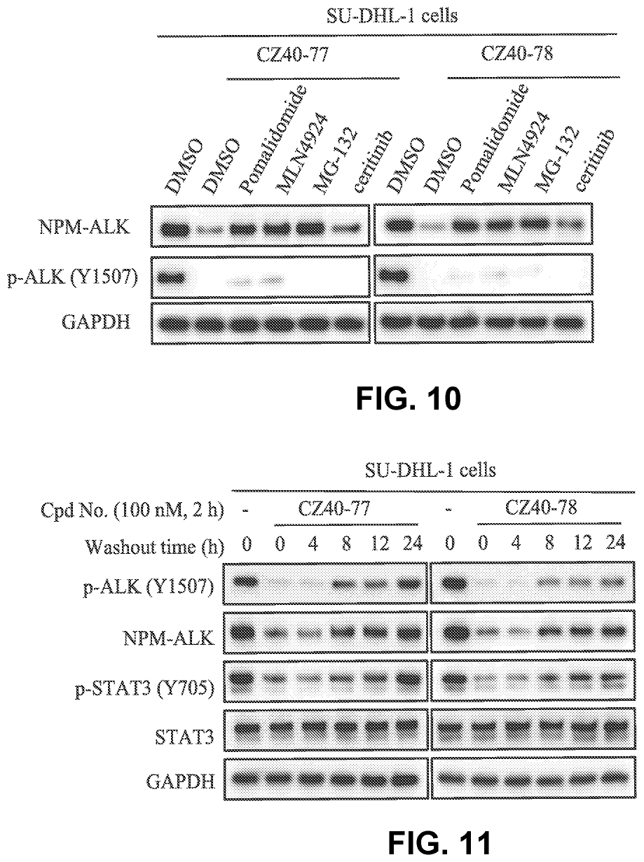

### Example 10. CZ40-77 and CZ40-78 Induced ALK Fusion Protein Degradation can be Rescued (FIG. 10)

SU-DHL-1 cells were pre-treated with DMSO, pomalidomide (10 μM), MLN4924 (1 μM), MG-132 (20 μM) or ceritinib (100 nM) for 2 h, before being treated with the 100 nM compounds for 6 h. CZ40-77 and CZ40-78 induced NPM-ALK degradation can be significantly rescued by pomalidomide. MLN4924, and MG-132.

### Example 11. CZ40-77 and CZ40-78 Induced ALK Fusion Protein Degradation can be Rescued (FIG. 10)

SU-DHL-1 cells were pre-treated with DMSO, pomalidomide (10 μM). MLN4924 (1 μM). MG-132 (20 μM) or ceritinib (100 nM) for 2 h, before being treated with the 100 nM compounds for 6 h. CZ40-77 and CZ40-78 induced NPM-ALK degradation can be significantly rescued by pomalidomide, MLN4924, and MG-132.

### Example 12. CZ40-77 and CZ40-78 Induced ALK Fusion Protein Degradation is Reversible (FIG. 11)

SU-DHL-1 cells were treated with DMSO or 100 nM compounds for 2 h, before being washed with PBS and incubated for the indicated length of time in fresh medium. The ALK fusion protein levels were recovered in 8 h.

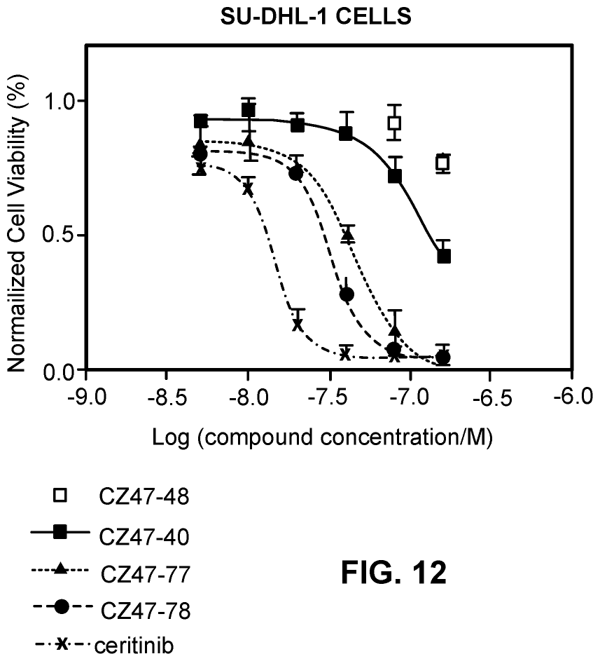

### Example 13. CZ40-77 and CZ40-78 Inhibited Viability of SU-DHL-1 Cells (FIG. 12)

SU-DHL-1 cells were seeded in 96-well plates at a density of 5000 cells per well, in triplicate. The cells were treated with DMSO or indicated serial dilutions of compounds for 3 days. Cell growth was measured using the CellTiter-Glo luminescent cell viability assay. Data were analyzed using the GraphPad Prism. CZ40-77 and CZ40-78 showed better cell viability inhibition than their negative controls CZ47-48 and CZ40-40.

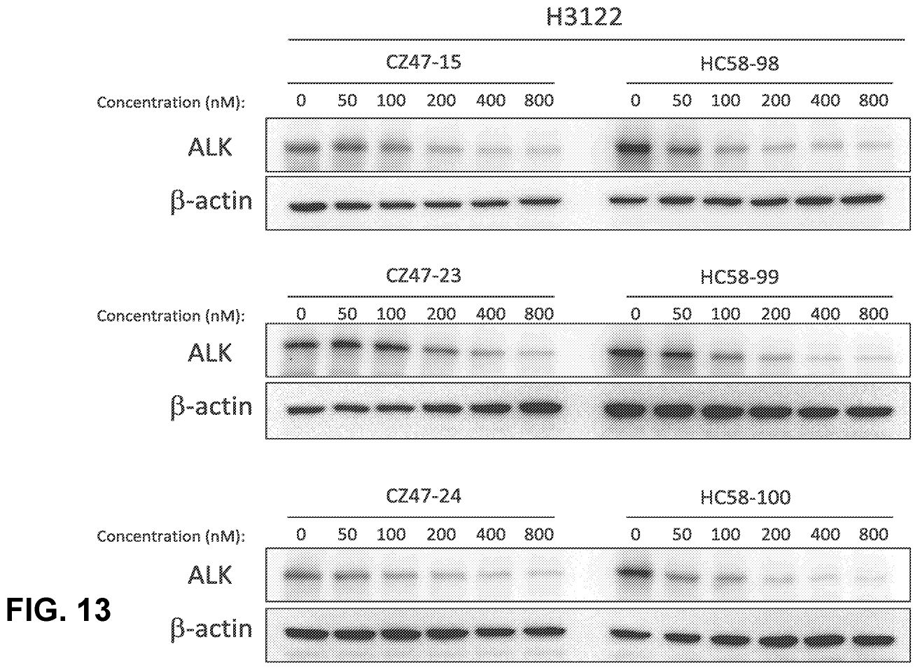

### Example 14. Selected ALK Degraders Significantly Reduced ALK Fusion Protein Levels in a Concentration-Dependent Manner in NCI-H3122 Cells (FIG. 13)

NCI-H3122 cells were treated with indicated compounds at 0, 50, 100, 200, 400, and 800 nM for 16 h. Western blot results showed that CZ47-15, CZ47-23, CZ47-24, HC58-98, HC58-99, and HC58-100 concentration-dependently reduced ALK fusion protein levels.

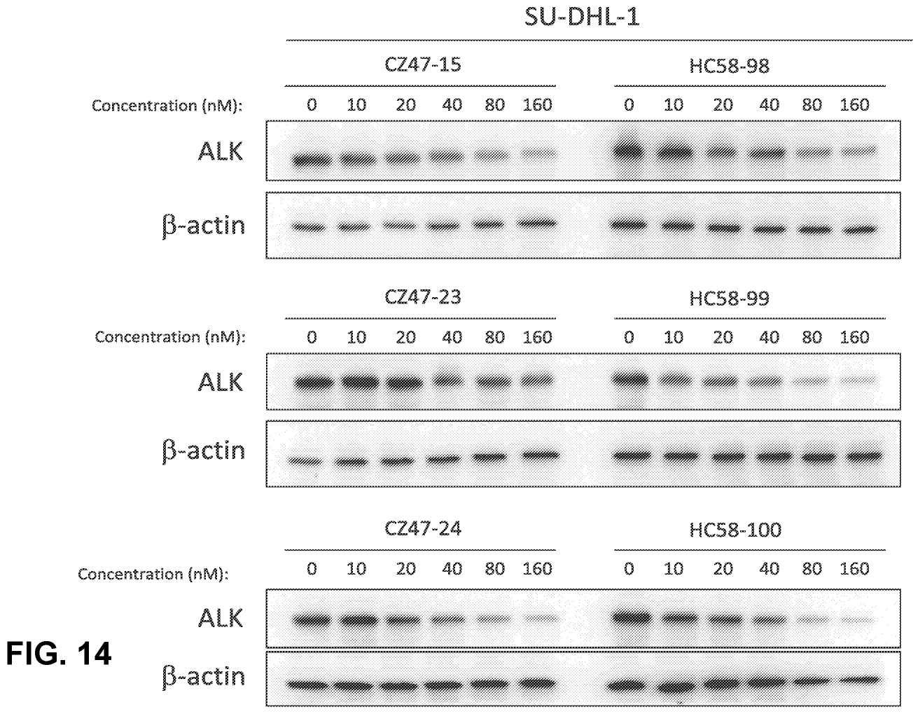

### Example 15. Selected ALK Degraders Significantly Reduced ALK Fusion Protein Levels in a Concentration-Dependent Manner in SU-DHL-1 Cells (FIG. 14)

SU-DHL-1 cells were treated with indicated compounds at 0, 10, 20, 40, 80, and 160 nM for 16 h. Western blot results showed that CZ47-15, CZ47-23, CZ47-24, HC58-98, HC58-99, and HC58-100 concentration-dependently reduced ALK fusion protein levels.

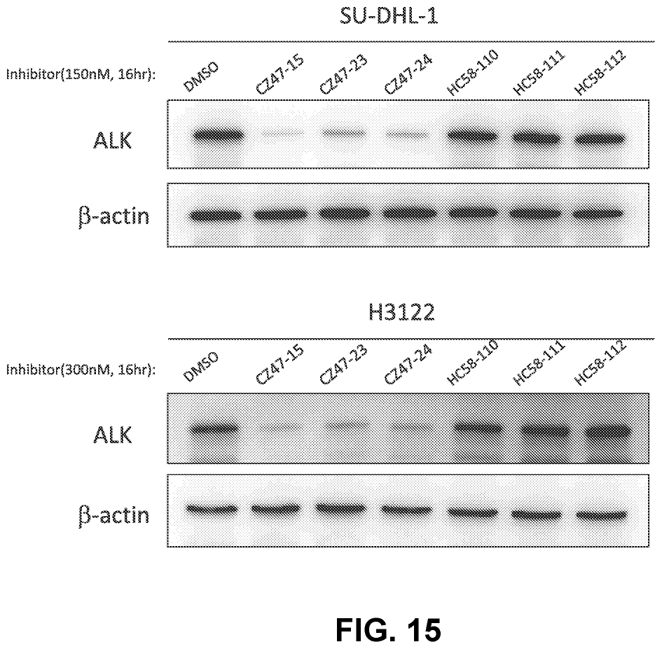

### Example 16. Selected ALK Degraders, but not their Negative Controls, Significantly Reduced ALK Fusion Protein Levels in SU-DHL-1 and NCI-H3122 Cells (FIG. 15)

SU-DHL-1 and NCI-H3122 cells were treated with DMSO, and indicated compounds at indicated concentrations for 16 h. Western blot results showed that CZ47-15, CZ47-23, and CZ47-24, but not their negative controls HC58-110, HC58-111, and HC58-112, significantly reduced ALK fusion protein levels.

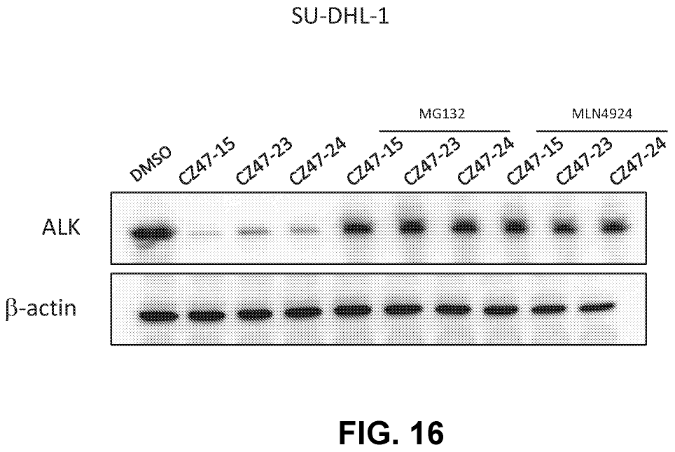

### Example 17. CZ47-15, CZ47-23, and CZ47-24 Induced ALK Fusion Protein Degradation can be Rescued (FIG. 16)

SU-DHL-1 cells were pre-treated with DMSO, MG-132 (20 μM), or MLN4924 (1 μM), for 2 h, before being treated with the 100 nM compounds for 6 h. CZ47-15, CZ47-23, and CZ47-24 induced ALK fusion protein degradation can be significantly rescued by MG-132 and MLN4924.

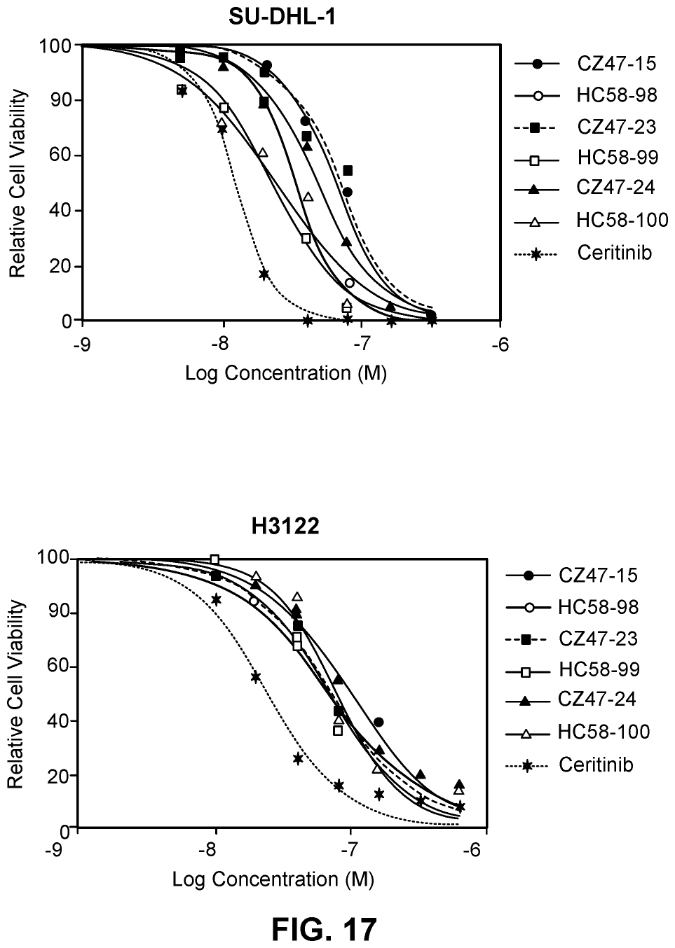

### Example 18. Selected ALK Degraders Inhibited Viability of SU-DHL-1 and NCI-H3122 Cells (FIG. 17)

SU-DHL-1 and NCI-3122 cells were treated with DMSO or indicated serial dilutions of compounds for 3 days. Data were analyzed using the GraphPad Prism. CZ47-15, CZ47-23, CZ47-24, HC58-98, HC58-99, and HC58-100 significantly inhibited cell viability of SU-DHL-1 and NCI-H3122 cells.

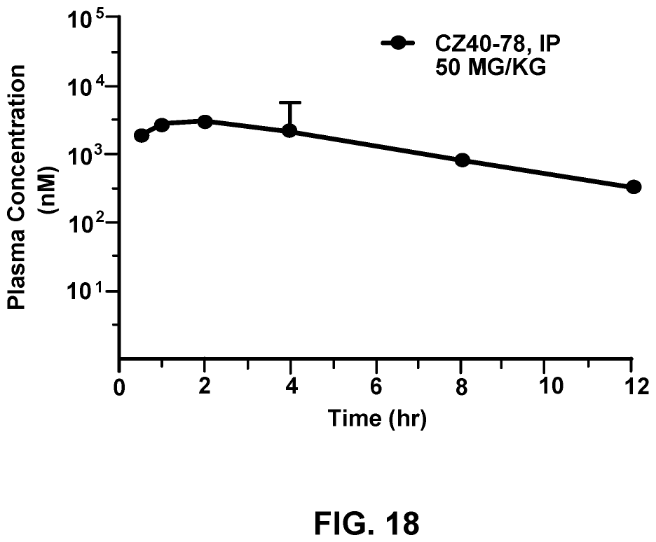

### Example 19. CZ40-78 is Bioavailable in Mice (FIG. 18)

Standard PK studies were conducted using male Swiss Albino mice. A single 50 mg/kg intraperitoneal (IP) injection of compound 6 (CZ40-78 of Table 1) was evaluated. Plasma concentrations of CZ40-78 reported at each of the 6 time points (30 mm, 1 h, 2 h, 4 h, 8 h, and 12 h post dosing) are the average values from 3 test animals. Error bars represent ±SD for triplicate samples.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Biisland, J. G., Wheeldon, A., Mead, A., Znamenskiy, P., Almond, S., Waters, K. A., Thakur, M., Beaumont, V., Bonnert, T. P., Heavens, R., et al. (2008). Behavioral and neurochemical alterations in mice deficient in anaplastic lymphoma kinase suggest therapeutic potential for psychiatric indications. Neuropsychopharmacology 33, 685-700.
Bondeson, D. P., Mares, A., Smith, I. E., Ko, E., Campos, S., Miah, A. H., Mulholland, K. E., Routly, N., Buckley, D. L., Gustafson, J. L., et al. (2015). Catalytic in vivo protein knockdown by small-molecule PROTACs. Nat Chem Biol 11, 611-617.
Buckley, D. L., and Crews, C. M. (2014). Small-molecule control of intracellular protein levels through modulation of the ubiquitin proteasome system. Angew Chem Int Ed Engl 53, 2312-2330.
Buckley, D. L., Gustafson, J. L., Van Molle, I., Roth, A G., Tae, H. S., Gareiss, P C., Jorgensen, W. L., Ciulli, A., and Crews, C. M. (2012a). Small-molecule inhibitors of the interaction between the E3 ligase VHL and HIF1alpha. Angew Chem Int Ed Engl 51, 11463-11467.
Buckley, D. L., Raina, K., Darricarrere, N., Hines, J., Gustafson, J. L., Smith, I. E., Miah, A. H., Harling, J. D., and Crews, C. M. (2015). HaloPROTACS: Use of Small Molecule PROTACs to Induce Degradation of HaloTag Fusion Proteins. ACS Chem Biol 10, 1831-1837.
Buckley, D. L., Van Molle, I., Gareiss, P. C., Tae, H. S., Michel, J., Noblin, D. J., Jorgensen, W. L., Ciulli, A., and Crews, C. M. (2012b). Targeting the von Hippel-Lindau E3 ubiquitin ligase using small molecules to disrupt the VHL/HIF-1 alpha interaction. J Am Chem Soc 134, 4465-4468.
Chamberlain, P. P., Lopez-Girona, A., Miller, K., Carmel, G., Pagarigan, B., Chie-Leon, B., Rychak, E., Corral, L. G., Ren, Y. J., Wang, M., et al. (2014). Structure of the human Cereblon-DDB1-lenalidomide complex reveals basis for responsiveness to thalidomide analogs. Nat Struct Mol Biol 21, 803-809.
Choi, Y. L., Soda, M., Yamashita. Y., Ueno, T., Takashima, J., Nakajima, T., Yatabe, Y., Takeuchi, K., Hamada, T., Haruta, H., et al. (2010). EML4-ALK mutations in lung cancer that confer resistance to ALK inhibitors. N Engl J Med 363, 1734-1739.
Choi, Y. L., Takeuchi, K., Soda, M., Inamura, K., Togashi, Y., Hatano, S., Enomoto, M., Hamada, T., Haruta, H., Watanabe, H., et al (2008). Identification of novel isoforms of the EML4-ALK transforming gene in non-small cell lung cancer. Cancer Res 68, 4971-4976.
Fischer, E. S., Bohm, K., Lydeard, J. R, Yang, H., Stadler, M. B., Cavadini, S., Nagel, J., Serluca, F., Acker, V., Lingaraju, G. M., et al. (2014). Structure of the DDB1-CRBN E3 ubiquitin ligase in complex with thalidomide. Nature 512, 49-53.
Galdeano, C., Gadd, M. S., Soares, P., Scaffidi, S., Van Molle, I., Birced, I., Hewitt, S., Dias, D. M., and Ciulli, A. (2014). Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663.
Hallberg, B., and Palmer, R. H. (2013). Mechanistic insight into ALK receptor tyrosine kinase in human cancer biology. Nat Rev Cancer 13, 685-700.
Ito, T., Ando, H., Suzuki, T., Ogura, T., Hotta, K., Imamura, Y., Yamaguchi, Y., and Handa, H. (2010). Identification of a primary target of thalidomide teratogenicity. Science 327, 1345-1350.
Iwahara, T., Fujimoto, J., Wen, D., Cupples, R, Bucay, N., Arakawa, T., Mori, S., Ratzkin, B., and Yamamoto, T. (1997). Molecular characterization of ALK, a receptor tyrosine kinase expressed specifically in the nervous system. Oncogene 14, 439-449.
Koivunen, J. P., Mermel, C., Zejnullahu, K., Murphy, C., Lifshits, E., Holmes, A. J., Choi, H. G., Kim, J., Chiang, D., Thomas, R., et al. (2008). EML4-ALK fusion gene and efficacy of an ALK kinase inhibitor in lung cancer. Clin Cancer Res 14, 4275-4283.
Kwak, E. L., Bang, Y. J., Camidge, D. R., Shaw, A. T., Solomon, B., Maki, R. G., Ou, S. H., Dezube, B. J., Janne, P. A., Costa, D. B., et al. (2010). Anaplastic lymphoma kinase inhibition in non-small-cell lung cancer. N Engl J Med 363, 1693-1703.
Lai, A. C., Toure, M., Hellerschmied, D., Salami, J., Jaime-Figueroa, S., Ko, E., Hines, J., and Crews, C. M. (2016). Modular PROTAC Design for the Degradation of Oncogenic BCR-ABL. Angew Chem Int Ed Engl 55, 807-810.
Lin, J. J., Riely, G. J., and Shaw, A. T. (2017). Targeting ALK: Precision Medicine Takes on Drug Resistance. Cancer Discov 7, 137-155.
Liu, Z., Wakihara, T., Oshima, K, Nishioka, D., Hotta, Y., Elangovan, S. P., Yanaba, Y., Yoshikawa, T., Chaikittisilp, W., Matsuo, T., et al. (2015). Widening Synthesis Bottlenecks: Realization of Ultrafast and Continuous-Flow Synthesis of High-Sihca Zeolite SSZ-13 for NOx Removal. Angew Chem Int Ed Engl 54, 5683-5687.
Lu, J., Qian, Y., Altieri, M., Dong, H., Wang, J., Raina, K., Hines, J., Winkler, J. D., Crew, A. P., Coleman, K., et al. (2015). Hijacking the E3 Ubiquitin Ligase Cereblon to Efficiently Target BRD4. Chemistry & biology 22, 755-763.
Morris, S. W., Kirstein, M. N., Valentine, M. B., Dittmer, K G., Shapiro, D. N., Saltman, D. L., and Look, A. T. (1994). Fusion of a kinase gene, ALK, to a nucleolar protein gene, NPM, in non-Hodgkin's lymphoma. Science 263, 1281-1284.
Morris, S. W., Naeve, C., Mathew, P., James, P. L., Kirstein, M. N., Cui, X., and Witte, D. P. (1997). ALK, the chromosome 2 gene locus altered by the t(2;5) in non-Hodgkin's lymphoma, encodes a novel neural receptor tyrosine kinase that is highly related to leukocyte tyrosine kinase (LTK). Oncogene 14, 2175-2188.
Peters, S., Camidge, D. R., Shaw, A. T., Gadgeel, S., Ahn, J. S., Kim, D. W., Ou, S. I., Perol, M., Dziadziuszko, R., Rosell, R., et al. (2017). Alectinib versus Crizotinib in Untreated ALK-Positive Non-Small-Cell Lung Cancer. N Engl J Med 377, 829-838.
Pulford, K, Lamant, L., Morris, S. W., Butler, L. H., Wood, K. M., Stroud, D., Delsol, G., and Mason, D. Y. (1997). Detection of anaplastic lymphoma kinase (ALK) and nucleolar protein nucleophosmin (NPM)-ALK proteins in normal and neoplastic cells with the monoclonal antibody ALK1 Blood 89, 1394-1404.
Rikova, K, Guo, A., Zeng, Q., Possemato, A., Yu, J., Haack, H., Nardone, J., Lee, K., Reeves, C., Li, Y., et al. (2007). Global survey of phosphotyrosine signaling identifies oncogenic kinases in lung cancer. Cell 131, 1190-1203.
Shaw, A. T., and Engelman, J. A. (2014). Ceritinib in ALK-rearranged non-small-cell lung cancer. N Engl J Med 370, 2537-2539.
Shiota, M., Fujimoto, J., Semba, T., Satoh, H., Yamamoto, T., and Mori, S. (1994). Hyperphosphorylation of a novel 80 kDa protein-tyrosine kinase similar to Ltk in a human Ki-1 lymphoma cell line, AMS3. Oncogene 9, 1567-1574.
Soda, M., Choi, Y. L., Enomoto, M., Takada, S., Yamashita, Y., Ishikawa, S., Fujiwara, S., Watanabe, H., Kurashina, K, Hatanaka, H., et al. (2007). Identification of the transforming EML4-ALK fusion gene in non-small-cell lung cancer. Nature 448, 561-566.
Solomon, B. J., Mok, T., Kim, D. W., Wu, Y. L., Nakagawa, K., Mekhail, T., Felip, E., Cappuzzo, F., Paolini, J., Usari, T., et al. (2014). First-line crizotinib versus chemotherapy in ALK-positive lung cancer. N Engl J Med 377, 2167-2177.
Takeuchi, K, Choi, Y. L., Togashi, Y., Soda, M., Hatano, S., Inamura, K, Takada, S., Ueno, T., Yamashita, Y., Satoh, Y., et al. (2009). KIF5B-ALK, a novel fusion oncokinase identified by an immunohistochemistry-based diagnostic system for ALK-positive lung cancer. Clin Cancer Res 75, 3143-3149.
Weiss, J. B., Xue, C., Benice, T., Xue, L., Morris, S. W., and Raber, J. (2012). Anaplastic lymphoma kinase and leukocyte tyrosine kinase: functions and genetic interactions in learning, memory and adult neurogenesis. Pharmacol Biochem Behav 100, 566-574.
Winter, G. E., Buckley, D. L., Paulk, J., Roberts, J. M., Souza. A., Dhe-Paganon, S., and Bradner, J. E. (2015). Phthalimide conjugation as a strategy for in vivo target protein degradation. Science 348, 1376-1381.
Wood, G. S., Hardman, D. L., Boni, R, Dummer, R, Kim, Y. H., Smoller, B. R., Takeshita, M., Kikuchi, M., and Burg, G. (1996). Lack of the t(2;5) or other mutations resulting in expression of anaplastic lymphoma kinase catalytic domain in CD30+ primary cutaneous lymphoproliferative disorders and Hodgkin's disease. Blood 88, 1765-1770.
Xie, T., Lim, S. M., Westover, K. D., Dodge, M. E., Ercan, D., Ficarro, S. B., Udayakumar, D., Gurbani, D., Tae, H. S., Riddle, S. M., et al. (2014). Pharmacological targeting of the pseudokinase Her3. Nat Chem Biol 10, 1006-1012.
Zengerle, M., Chan, K. H., and Ciulli, A. (2015). Selective Small Molecule Induced Degradation of the BET Bromodomain Protein BRD4. ACS Chem Biol 10, 1770-1777.

uptors to treat ALK-mediated cancer are provided.

1. A bivalent compound comprising an anaplastic lymphoma kinase (ALK) ligand conjugated to a degradation/disruption tag through a linker, said linker selected from the group consisting of: 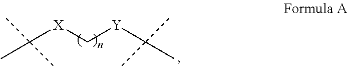
wherein
X is C═O or CH₂;
Y is C═O or CH₂; and
n is 0-15; 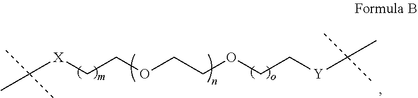
wherein
X is C═O or CH₂;
Y is C═O or CH₂;
m is 0-15;
n is 0-6; and
o is 0-15; and 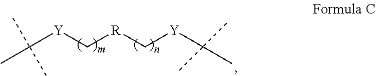
wherein
X is C═O or CH₂;
Y is C═O or CH₂;
R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl)-, —C═C—, —O—, —NH—, —N(C₁₋₃ alkyl)-, —C(O)NH—, —C(O)N(C₁₋₃ alkyl)-, a 3-13 membered ring, a 3-13 membered fused ring, a 3-13 membered bridged ring, and/or a 3-13 membered spiro ring;
m is 0-15; and
n is 0-15,
and enantiomers and pharmaceutically acceptable derivatives thereof.

2. The bivalent compound of claim 1, wherein the linker is selected from the group consisting of: 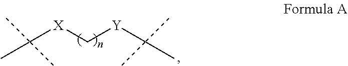
wherein
X is C═O or CH₂;
Y is C═O or CH₂; and
n is 0-15.

3. The bivalent compound of claim 1, wherein the linker is selected from the group consisting of: 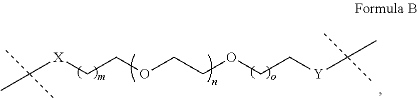
wherein
X is C═O or CH₂;
Y is C═O or CH₂;
m is 0-15;
n is 0-6; and
o is 0-15.

4. (canceled)

5. (canceled)

6. (canceled)

7. (canceled)

8. (canceled)

9. (canceled)

10. The bivalent compound of claim 1, wherein the linker is selected from the group consisting of: 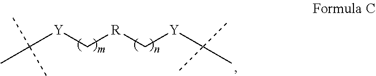
wherein
X is C═O or CH₂;
Y is C═O or CH₂;
R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl)-, —C═C—, —O—, —NH—, —N(C₁₋₃ alkyl)-, —C(O)NH—, —C(O)N(C₁₋₃ alkyl)-, a 3-13 membered ring, a 3-13 membered fused ring, a 3-13 membered bridged ring, and/or a 3-13 membered spiro ring;
m is 0-15; and
n is 0-15.

11. (canceled)

12. (canceled)

13. (canceled)

14. (canceled)

15. The bivalent compound of claim 10, wherein the linker is Formula C and R is selected from the group consisting of:

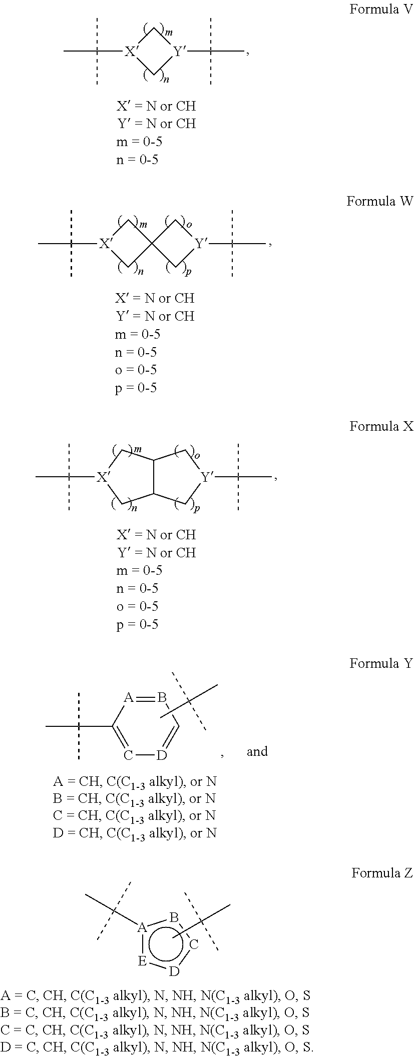

16. (canceled)

17. (canceled)

18. The bivalent compound of claim 1, wherein the ALK ligand is selected from the group consisting of: 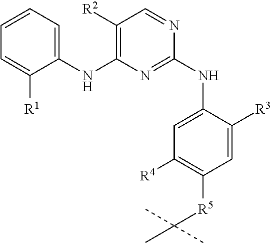
wherein
R¹ is (CR⁶R⁷)ₙSO₂R⁸, (CR⁶R⁷)ₙSO₂NR⁸R⁹, (CR⁶R⁷)ₙCOR⁸, (CR⁶R⁷)ₙCO₂R⁸, (CR⁶R⁷)ₙCONR⁸R⁹, (CR⁶R⁷)ₙP(O)R⁸R⁹, (CR⁶R⁷)ₙP(O)NR⁸R⁹;
R², R³ and R⁴ are independently hydrogen, halogen, C1-C8 alkyl, C1-C8 alkoxy, C1-C8 alkoxy alkyl;
R⁵ are independently hydrogen, halogen, C1-C8 alkyl, C1-C8 alkoxy, C1-C8 alkoxyalkyl, C1-C8 haloalkyl, C1-C8 hydroxyalkyl, C3-C7 cycloalkyl, C3-C7 heterocyclyl, C2-C8 alkenyl, C2-C8 alkynyl, OR¹⁰, SR¹⁰, NR¹⁰R¹¹, CN, NO₂, (CR¹⁰R¹¹)mNR¹²R¹³, (CR¹⁰R¹¹)mC(O)R¹², (NR¹⁰R¹¹)mNR¹²R¹³, (NR¹⁰R¹¹)mC(O)R¹², COR¹⁰, CO₂R¹⁰, CONR¹⁰R¹¹, NR¹⁰COR¹¹, NR¹⁰SOR¹¹, NR¹⁰SO₂R¹¹, SOR¹⁰, SO₂R¹⁰, SO₂NR¹⁰R¹¹, (CR¹⁰R¹¹)m-aryl, or (CR¹⁰R¹¹)m-heteroaryl;
m=0-8;
n=0-3;
R⁶, R⁷, R⁸, R⁹, R¹⁰, R¹¹, R¹², R¹³ are independently hydrogen, C1-C8 alkyl, C1-C8 alkoxy, C2-C8 alkenyl, C2-C8 alkynyl, arylalkyl, cycloalkyl, heterocycloalkyl, aryl, heteroaryl, or heteroarylalkyl; and
R⁶ and R⁷, R⁸ and R⁹, R¹⁰ and R¹¹, R¹² and R¹³ independently form 4-8 membered alkyl or heterocyclyl rings.

19. The bivalent compound of claim 1, wherein the ALK ligand is selected from the group consisting of:

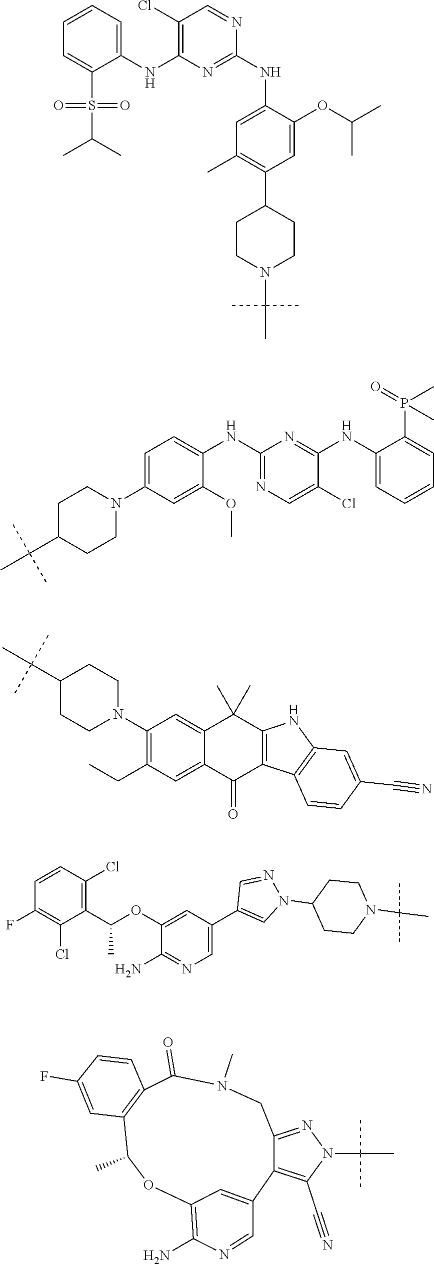

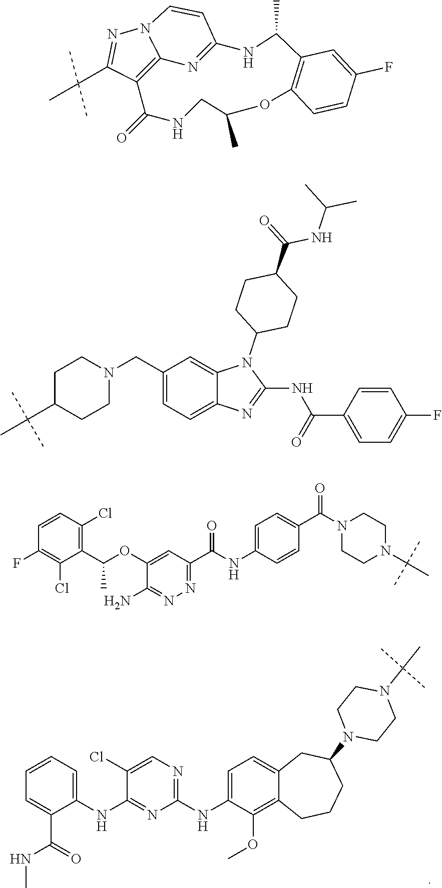

20. The bivalent compound of claim 1, wherein the ALK ligand is selected from the group consisting of crizotinib, ceritinib, alectinib, brigatinib, lorlatinib, TPX-0005, belizatinib, ensartinib, CEP-37440, and analogs thereof.

21. The bivalent compound of claim 20, wherein the ALK ligand is ceritinib.

22. The bivalent compound of claim 1, wherein the degradation/disruption tag is selected from the group consisting of: 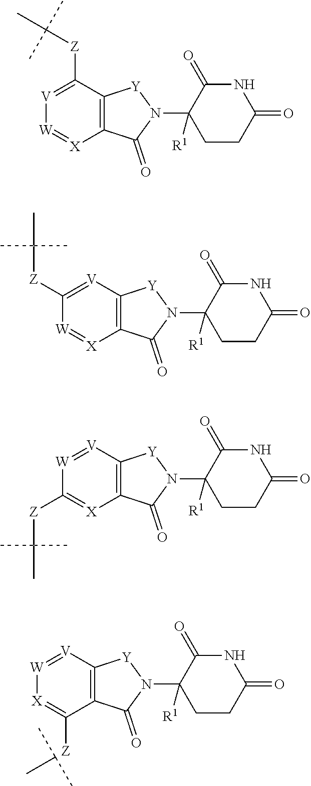
where
V, W, X are independently CR², or N;
Y is CO or CH₂;
Z is CH₂, NH, or O;
R¹ is hydrogen, methyl, or fluoro;
R² is hydrogen, halogen, or C1-C5 alkyl; 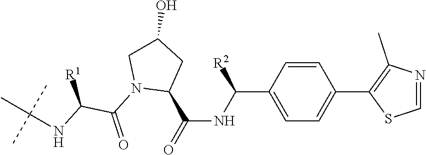
wherein
R¹ and R² are independently hydrogen, C1-C8 alkyl, C1-C8 alkoxyalkyl, C1-C8 haloalkyl, C1-C8 hydroxyalkyl, C3-C7 cycloalkyl, C3-C7 heterocyclyl, C2-C8 alkenyl, or C2-C8 alkynyl; and 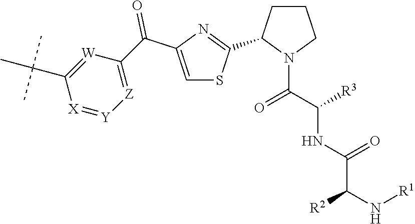
wherein
R¹, R², R³ and R⁴ are independently hydrogen, C1-C8 alkyl, C1-C8 alkoxyalkyl, C1-C8 haloalkyl, C1-C8 hydroxyalkyl, C3-C7 cycloalkyl, C3-C7 heterocyclyl, C2-C8 alkenyl, or C2-C8 alkynyl; and
V, W, X, Z are independently CR⁴, or N.

23. The bivalent compound of claim 1, wherein the degradation/disruption tag is selected from the group consisting of:

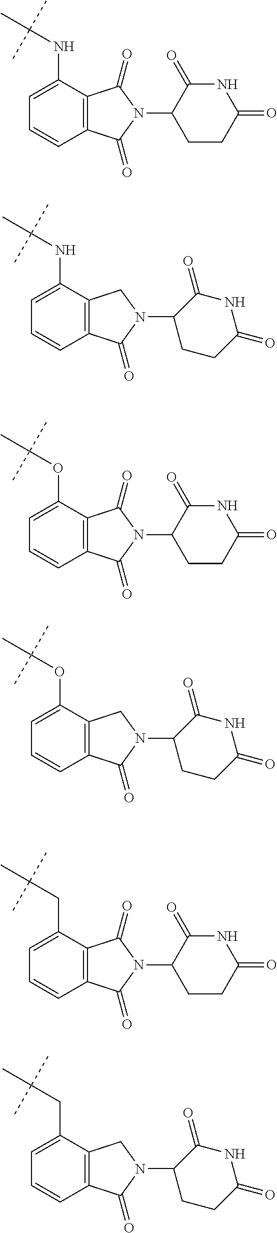

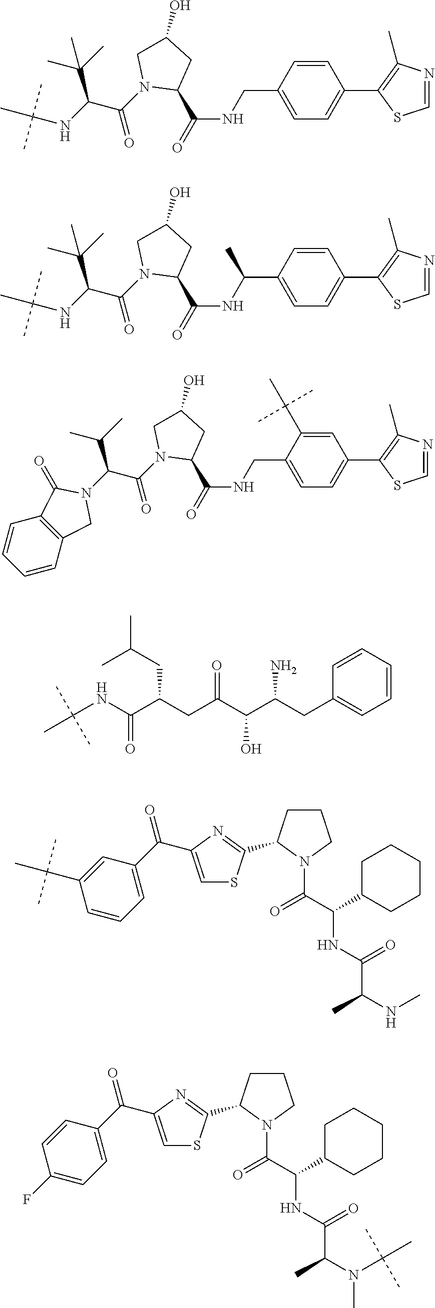

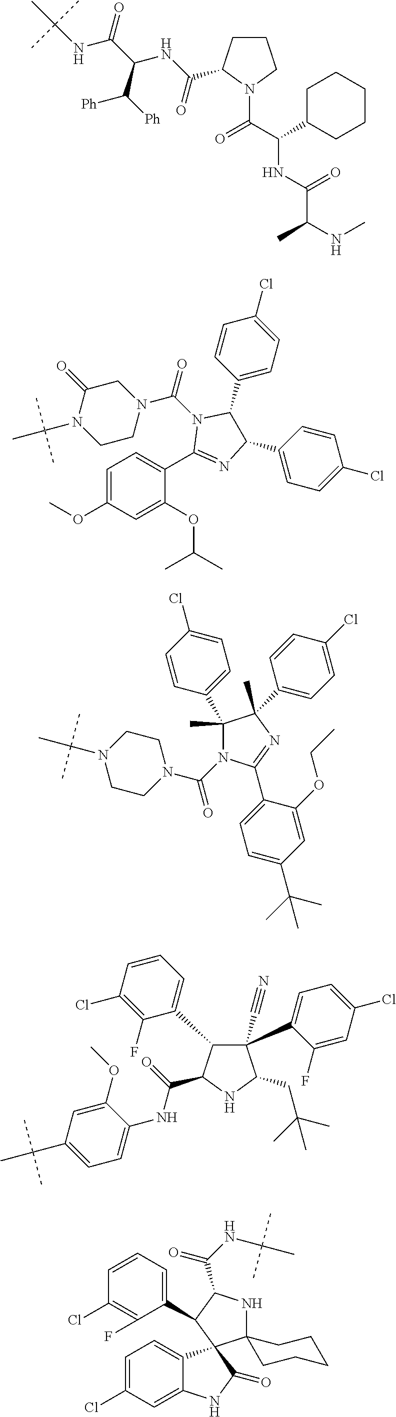

24. The bivalent compound of claim 1, wherein the degradation/disruption tag binds to an ubiquitin ligase selected from the group consisting of cereblon E3 ligase, VHL E3 ligase, MDM2 ligase, TRIM21 ligase, TRIM24 ligase, and IAP ligase or the degradation/disruption tag is a hydrophobic group that leads to ALK protein misfolding.

25. The bivalent compound of claim 24, wherein the ubiquitin ligase is an E3 ligase.

26. The bivalent compound of claim 25, wherein the E3 ligase is selected from the group consisting of cereblon E3 ligase, VHL E3 ligase, MDM2 ligase, TRIM24 ligase, TRIM21 ligase, and IAP ligase.

27. The bivalent compound of claim 1, wherein the degradation/disruption tag is selected from the group consisting of pomalidomide, thalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, and analogs thereof.

28. (canceled)

29. (canceled)

30. (canceled)

31. (canceled)

32. A compound selected from the group consisting of:
2-(4-(4-((5-Chloro-4-((2-(isopropylsulfonyl)phenyl)amino)pyrimidin-2-yl)amino)-5-isopropoxy-2-methylphenyl)piperidin-1-yl)-N-(14-((2-(2,6-dioxopiperidin-3-yl)-1,3-dioxoisoindolin-4-yl)amino)-3,6,9,12-tetraoxatetradecyl)acetamide (CZ40-53);
(2S,4R)-1-((S)-14-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-(tert-butyl)-4,14-dioxo-6,9,12-trioxa-3-azatetradecanoyl)-4-hydroxy-N-(4-(4-methylthiazol-5-yl)benzyl)pyrrolidine-2-carboxamide (CZ40-77);
2-(4-(4-((5-Chloro-4-((2-(isopropylsulfonyl)phenyl)amino)pyrimidin-2-yl)amino)-5-isopropoxy-2-methylphenyl)piperidin-1-yl)-N-(2-((2-(2,6-dioxopiperidin-3-yl)-1,3-dioxoisoindolin-4-yl)amino)ethyl)acetamide (CZ40-78);
(2S,4R)-1-((S)-2-(2-(2-(2-(4-(4-((5-Chloro-4-((2-(isopropyl sulfonyl)phenyl)amino)pyrimidin-2-yl)amino)-5-isopropoxy-2-methylphenyl)piperidin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide (CZ47-15);
(2S,4R)-1-((S)-2-(5-(4-(4-((5-Chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-4-hydroxy-N-(4-(4-methylthiazol-5-yl)benzyl)pyrrolidine-2-carboxamide (CZ47-23);
(2S,4R)-1-((S)-2-(6-(4-(4-((5-Chloro-4-((2-(isopropylsulfonyl)phenyl)amino)pyrimidin-2-yl)amino)-5-isopropoxy-2-methylphenyl)piperidin-1-yl)-6-oxohexanamido)-3,3-dimethylbutanoyl)-4-hydroxy-N-(4-(4-methylthiazol-5-yl)benzyl)pyrrolidine-2-carboxamide (CZ47-24);
(2S,4R)-1-((S)-2-(2-(2-(2-(4-(4-((5-chloro-4-((2-(isopropylsulfonyl)phenyl)amino)pyrimidin-2-yl)amino)-5-isopropoxy-2-methylphenyl)piperidin-1-yl)-2-oxoethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (HC58-98);
(2S,4R)-1-((S)-2-(5-(4-(4-((5-chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (HC58-99); and
(2S,4R)-1-((S)-2-(6-(4-(4-((5-chloro-4-((2-(isopropylsulfonyl)phenyl)amino)pyrimidin-2-yl)amino)-5-isopropoxy-2-methylphenyl)piperidin-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (HC58-100),
and enantiomers and pharmaceutically acceptable derivatives thereof.

33. A method of treating an anaplastic lymphoma kinase (ALK)-mediated cancer, which comprises administering to a subject with an ALK-mediated cancer and in need thereof, a bivalent compound according to claim 1.

34. (canceled)

35. (canceled)

36. (canceled)

37. The method of claim 33, wherein at least one bivalent compound is administered orally, parenterally, intradermally, subcutaneously, topically, or rectally.

38. The method of claim 33, further comprising treating the subject with one or more additional therapeutic regimens for treating cancer.

39. (canceled)

40. (canceled)

41. (canceled)

42. (canceled)

43. A method for identifying a bivalent compound which mediates degradation/disruption of ALK, the method comprising:
providing a heterobifunctional test compound comprising a ALK ligand conjugated to a degradation/disruption tag through a linker;
contacting the heterobifunctional test compound with a cell comprising a ubiquitin ligase and ALK;
determining whether ALK levels decrease in the cell; and
identifying the heterobifunctional test compound as a bivalent compound which mediates degradation/reduction of ALK levels decrease in the cell

44. (canceled)

45. (canceled)
